(19) United States
(12) Patent Application Publication
(10) Pub. No.: US 2022/0233716 A1
(43) Pub. Date: Jul. 28, 2022

(54) METHODS OF VIRAL DELIVERY TO A POPULATION OF CELLS

(71) Applicant: Avectas Limited, Co. Kildare (IE)

(72) Inventors: Michael Maguire, Dublin (IE); Shirley O'Dea, Maynooth (IE)

(21) Appl. No.: 17/615,081

(22) PCT Filed: May 30, 2020
(86) PCT No.: PCT/IB20/55148

§ 371 Date: Nov. 29, 2021

Related U.S. Application Data
(60) Provisional application No. 62/855,241, filed on May 31, 2019.

(51) Int. Cl. A61K 48/00; C12N 15/86; C12N 15/87

(52) U.S. Cl. CPC A61K 48/005; C12N 2740/16041; C12N 15/87; C12N 15/86

(57) ABSTRACT

The invention provides a method of viral transfection of cells.

### RELATED APPLICATIONS

This application claims the benefit of priority under 35 U.S.C. § 119(e) to U.S. Provisional Application No. 62/855,241 filed May 31, 2019, the entire contents of each of which is incorporated herein by reference in their entireties.

### FIELD OF THE INVENTION

The invention relates to the delivery of agents (e.g., viruses) into mammalian cells and productions of viruses thereof.

### BACKGROUND OF THE INVENTION

Viruses are widely used as effective gene-delivery vehicles. Transduction is the process by which foreign DNA is introduced into a cell by a virus or viral vector.

Transduction is a common tool used by molecular biologists to stably introduce a foreign gene into a host cell, e.g., mammalian, genome. Optimization of the conditions for transduction is of a high importance for a variety of research applications and clinical applications. For hard to transfect cells, lentivirus transduction offers a high efficiency solution for attaining good expression levels. Efficiency, time, production costs, and cell viability remain challenges in the field.

### SUMMARY OF THE INVENTION

The invention provides a solution to the problem of delivering a complex cargo such as viruses into cells. A virus is an infective agent that typically consists of a nucleic acid molecule in a protein coat. A virus is a microorganism that is smaller than a bacterium that cannot grow or reproduce apart from a living cell, i.e., a virus is able to multiply only within the living cells of a host.

The invention features a method of delivering a virus across a plasma membrane of a cell, comprising the steps of providing a population cells and contacting the population of cells with a volume of an isotonic aqueous solution comprising the virus and an alcohol at a concentration of greater than 2%. The contacting of the population of cells with the volume of aqueous solution is performed by gas propelling the aqueous solution to form a spray. The spray comprises a droplet (or a population of droplets) comprising a diameter of greater than or equal to 150 μm. For example, the spray comprises a droplet (or a population of droplets) comprising a diameter in the range of 177 μm to 590 μm.

Exemplary viruses to be delivered include a lentivirus, a retrovirus, an adenovirus, an adeno-associated virus (AAV), or a herpes simplex virus (HSV). In preferred embodiments, the virus is a lentivirus.

The population of cells comprises mammalian cells, e.g., human immune cells. The population of cells may comprise adherent or suspension cells. In some examples, the population of cells comprises a non-adherent cell such as a T lymphocyte or a natural killer (NK) cell. The population of cells may comprises primary cells or cell lines. For example, the population comprises HEK293 cells, HEK293T cells, Lenti-x 293T cells, or HEK293F cells. The method yields a transduction efficiency of the cells that is at least 30%, at least 40%, at least 50%, or at least 60%.

The aqueous solution comprises an alcohol such as ethanol. For example, the aqueous solution comprises greater than 2% ethanol, greater than 10% ethanol, e.g., the aqueous solution comprises between 20-30% ethanol. An exemplary solution comprises an ethanol concentration of 5 to 30%.

The aqueous solution further comprises one or more of the following components: 75 to 98% H2O, 2 to 45% ethanol, 6 to 91 mM sucrose, 2 to 35 mM KCl, 2 to 35 mM ammonium acetate, and 1 to 14 mM (4-(2-hydroxyethyl)-1-piperazineethanesulfonic acid) (HEPES). For example, the solution comprises: Sucrose 32.5 mM, KCl 106 mM, Hepes 5 mM, and Ethanol 12% v/v.

To receive the spray solution containing the cargo, e.g., virus, to be delivered, the population of cells may be present as a layer, e.g. a monolayer, of non-adherent cells on a substrate. The layer may be confluent or non-confluent. An exemplary layer of cells, e.g., a monlayer resides on a membrane filter.

The invention provides a solution to the problem of delivering viruses and compositions into cells. Accordingly, a method of delivering viruses across a plasma membrane of a cell (e.g., suspension or adherent cells) comprises the steps of providing a population of and contacting the population of cells with a volume of an isotonic aqueous solution, the aqueous solution including the virus.

The aqueous solution for delivering virus to cells comprises a salt, e.g., potassium chloride (KCl) in between 12.5-500 mM. For example, the solution is isotonic with respect to the cytoplasm of a mammalian cell such as a human T cell. Such an exemplary isotonic delivery solution contains 106 mM KCl.

The methods are used to deliver any virus to mammalian cells, adherent or non-adherent and are particularly useful to deliver cargo to non-adherent cells because of the difficulties associated with doing so prior to the invention. In some examples, the non-adherent cell comprises a peripheral blood mononuclear cell, e.g., the non-adherent cell comprises an immune cell such as a T cell (T lymphocyte), e.g., an activated or non-activated (a.k.a., naïve) T cell. An immune cell such as a T cell is optionally activated with a ligand of CD3, CD28, or a combination thereof. For example, the ligand is an antibody or antibody fragment that binds to CD3 or CD28 or both.

The method involves delivering the virus in the delivery solution to a population of non-adherent cells comprising a monolayer, e.g., a sheet of cells physically located on a support or substrate. For example, the cells form a layer, which is contacted with a spray of aqueous delivery solution. For example, the monolayer (or layer) is contacted with a spray of aqueous delivery solution. The method delivers the virus (compound or composition) into the cytoplasm of the cell and wherein the population of cells comprises a maintains a high percent viability following the procedure. The method also delivers the virus (e.g., compound or composition) in the form of a spray, wherein the spray is a low volume. The low volume of the spray concentrates the virus at the plasma membrane of the cells.

In certain embodiments, the monolayer of non-adherent/suspension cells resides on a membrane filter. In some examples, the membrane filter is moved, e.g., agitated or vibrated, following contacting the cell monolayer with a spray of the delivery solution. The membrane filter may be vibrated or agitated before, during, and/or after spraying the cells with the delivery solution.

The delivery solution includes an isotonic aqueous solution, the aqueous solution including the payload and an alcohol at greater than 2 percent (v/v) concentration. The alcohol can comprise ethanol. The aqueous solution can comprise greater than 10% ethanol. The aqueous solution can comprise between 20-30% ethanol. The aqueous solution can comprise 27% ethanol. The aqueous solution can comprise between 12.5-500 mM KCl. The aqueous solution can comprise of 106 mM KCl. In embodiments, the aqueous solution comprises 27% ethanol. In other examples, the aqueous solution comprises 12% ethanol. In examples, the aqueous solution comprises 32.5 mM, potassium chloride (KCl) 106 mM, Hepes 5 mM, ethanol, (EtOH) 12% v/v, and water for injection (WFI) In examples, “S Buffer” includes a hypotonic physiological buffered solution (78 mM sucrose, 30 mM KCl, 30 mM potassium acetate, 12 mM HEPES) for 5 min at 4° C. (Medepalli K. et al., Nanotechnology 2013; 24(20); incorporated herein by reference in its entirety). In some examples, potassium acetate is replaced with ammonium acetate in the S Buffer. S buffer is further described in international application WO 2016/065341, e.g., at ¶ [0228]-[0229] and incorporated herein by reference in its entirety.

The non-adherent cells can comprise a peripheral blood mononuclear cell. The non-adherent cells can comprise an immune cell. The non-adherent cells can comprise T lymphocytes. The population of non-adherent cells can comprise a monolayer.

In another aspect, a composition comprises an isotonic aqueous solution, the aqueous solution comprising KCl at a concentration of 10-500 mM and ethanol at greater than 5 percent (v/v) concentration for use to deliver a cargo compound or composition to a mammalian cell. The KCl concentration can be 106 mM and said alcohol concentration can be 27%. In embodiments, the aqueous solution comprises 27% ethanol for the Flexi (e.g., small scale). In embodiments the aqueous solution comprises 12% ethanol in a large scale system.

In aspects, provided herein are methods for delivering a virus across a plasma membrane of a non-adherent cell, the method comprising, providing a population of non-adherent cells; and contacting the population of cells with a volume of an isotonic aqueous solution, wherein the aqueous solution includes the virus. In embodiments, the virus comprises a retrovirus, lentivirus, an adenovirus, an adeno-associated virus (AAV), or a herpes simplex virus (HSV).

In embodiments, the population of cells comprises adherent or suspension cells. For example, the population of cells comprises HEK293 cells, HEK293T cells, Lenti-x 293T cells, or HEK293F cells.

Also provided herein are methods for delivering a virus across a plasma membrane of a non-adherent cell, the method comprising, providing a population of non-adherent cells; and contacting the population of cells with a volume of an isotonic aqueous solution, the aqueous solution including the virus and an alcohol at greater than 2 percent (v/v) concentration. In embodiments, the virus comprises a retrovirus, lentivirus, an adenovirus, an adeno-associated virus (AAV), or a herpes simplex virus (HSV).

In embodiments, the population of cells comprises adherent or suspension cells, for example, HEK293 cells, HEK293T cells, Lenti-x 293T cells, or HEK293F cells.

In embodiments, the transfection (or transduction) efficiency is at least 20%, at least 30%, at least 40%, at least 50%, or at least 60% (or higher %).

In embodiments, the alcohol comprises ethanol. In other embodiments, the aqueous solution comprises greater than 10% ethanol. In embodiments, the aqueous solution comprises between 20-30% ethanol. In embodiments, the aqueous solution comprises 27% ethanol. In embodiments the aqueous solution comprises 12% ethanol, e.g., in a larger scale system.

In embodiments, the population of cells comprises a monolayer of non-adherent cells. In examples, the monolayer is contacted with a spray of said aqueous solution. The mono layer may further reside on a membrane filter.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

FIG. 1 is a schematic showing an overview of a viral production protocol. As noted by the arrow, the methods described herein are incorporated into the plasmid transfection step of the protocol.

FIG. 2 is an image showing viral entry pathways, from Biophys J 2016 Mar. 8; 110(5): 1028-1032, incorporated herein by reference in its entirety.

FIG. 3 is a bar graph showing the viability of T cells before and after the delivery of lentiviral (LV)-GFP.

FIG. 4 is a bar graph showing the cumulative fold expansion up to 96 hr after infection.

FIG. 5 is a bar graph showing GFP expression in T cells at day 3 and day 4 following LV-GFP delivery.

FIGS. 6A-6D depict representative 4× images of formulated Delivery Solution with LV-eGFP (enhanced GFP) after 1 hr (FIG. 6A), 2 hr (FIG. 6B), 3 hr (FIG. 6C), and 4 hr (FIG. 6D) at room temperature.

FIGS. 7A and 7B depict histogram profiles of CD3 (FIG. 7A) and CD3+CD25 (FIG. 7B) expression of day 3 PBMC-initiated T-cells.

FIG. 8 is a bar graph showing cell recovery after the SOLUPORE™ process with the system (n=3 technical replicates).

FIGS. 9A and 9B are line graphs showing the viability (FIG. 9A) and cumulative fold expansion (FIG. 9B) up to 3 days after viral delivery by the SOLUPORE™ process or static transduction (n=3 technical replicates). Statistical analysis calculated using a paired two-tailed t-test (* p<0.05, ** p<0.01, *** p<0.001).

FIGS. 10A and 10B are line graphs showing GFP expression (FIG. 10A) and median fluorescence intensity (FIG. 10B) up to 3 days after viral delivery by the SOLUPORE™ process or static transduction (n=3 technical replicates). Statistical analysis calculated using a paired two-tailed t-test (* p<0.05, ** p<0.01, ***p<0.001).

FIGS. 11A-11B are images of histogram profiles of CD3 (FIG. 11A) and CD3+CD25 (FIG. 11B) expression of day 3 PBMC-initiated T-cells.

FIG. 12 is a bar graph showing cell recovery after the SOLUPORE™ process with the system (n=3 technical replicates) described herein.

FIGS. 13A and 13B are line graphs showing the viability (FIG. 13A) and cumulative fold expansion (FIG. 13B) up to 4 days after viral delivery by the SOLUPORE™ process or static transduction (n=3 technical replicates). Statistical analysis calculated using a paired two-tailed t-test (* p<0.05, ** p<0.01, *** p<0.001).

FIGS. 14A and 14B are line graphs showing GFP expression in % (FIG. 14A) and median fluorescence intensity (FIG. 14B) up to 4 days after viral delivery by the SOLUPORE™ process or static transduction (n=3 technical replicates). Statistical analysis calculated using a paired two-tailed t-test (* p<0.05, ** p<0.01, ***p<0.001).

FIGS. 15A and 15B are images showing histogram profiles of CD3 (FIG. 15A) and CD3+CD25 (FIG. 15B) expression of day 3 PBMC-initiated T-cells.

FIG. 16 is a bar graph showing cell recovery after the SOLUPORE™ process with the system (n=6).

FIGS. 17A and 17B are line graphs showing viability up to 3 days after viral delivery by the SOLUPORE™ process or static transduction (n=3 technical replicates). Impact on viability due to viral delivery method (FIG. 17A) and MOI of LV (FIG. 17B) are shown. Statistical analysis calculated using a two-way ANOVA (* p<0.05, ** p<0.01, *** p<0.001).

FIGS. 18A and 18B are line graphs showing the cumulative fold expansion up to 4 days after viral delivery by the SOLUPORE™ process or static transduction (n=3 technical replicates). Impact on expansion due to viral delivery method (A) and MOI of LV (B) are shown. Statistical analysis calculated using a two-way ANOVA (* p<0.05, ** p<0.01, *** p<0.001).

FIGS. 19A-19B are line graphs showing the impact of viral delivery method on GFP expression in % (FIG. 19A) and in median fluorescence intensity (FIG. 19B) up to 4 days post-infection. Impact of MOI delivered on % GFP expression (FIG. 19C), as well as GFP MFI up to 4 days post-infection (FIG. 19D) are shown. N=3 technical replicates for each MOI condition tested. Statistical analysis calculated using a two-way ANOVA (* p<0.05, ** p<0.01, ***p<0.001).

FIGS. 20A and 20B are images of Histogram profiles of CD3 (FIG. 20A) and CD3+CD25 (FIG. 20B) expression of day 3 PBMC-initiated T-cells.

FIG. 21 is a bar graph showing cell recovery after the SOLUPORE™ process with the system (n=6).

FIGS. 22A and 22B are line graphs showing Viability up to 4 days after viral delivery by the SOLUPORE™ process or static transduction (n=3 technical replicates). Impact on viability due to viral delivery method (FIG. 22A) and MOI of LV (FIG. 22B) are shown. Statistical analysis calculated using a two-way ANOVA (* p<0.05, ** p<0.01, *** p<0.001).

FIGS. 23A and 23B are line graphs showing Cumulative fold expansion up to 4 days after viral delivery by the SOLUPORE™ process or static transduction (n=3 technical replicates). Impact on expansion due to viral delivery method (FIG. 23A) and MOI of LV (FIG. 23B) are shown. Statistical analysis calculated using a two-way ANOVA (* p<0.05, ** p<0.01, *** p<0.001).

FIGS. 24A-24D are line graphs showing the Impact of viral delivery method on GFP expression in % (FIG. 24A) and in median fluorescence intensity (FIG. 24B) up to 4 days post-infection. Impact of MOI delivered on % GFP expression (FIG. 24C), as well as GFP MFI up to 4 days post-infection (FIG. 24D) are shown. N=3 technical replicates for each MOI condition tested. Statistical analysis calculated using a two-way ANOVA (* p<0.05, ** p<0.01, ***p<0.001).

FIG. 25 is a bar graph showing the expression of CD3 and CD25 of day 3 PBMC initiated T-cells (n=4).

FIG. 26 is a bar graph showing cell recovery after the SOLUPORE™ process with the system (n=18).

FIGS. 27A-27C are line graphs showing the viability up to 4 days after viral delivery by the SOLUPORE™ process or static transduction (n=18). Impact on viability due to viral delivery method (FIG. 27A), MOI of LV (FIG. 27B) and changes made between Run 1/2 and Run 3/4 (FIG. 27C) are shown. Statistical analysis calculated using a three-way ANOVA (* p<0.05, ** p<0.01, *** p<0.001).

FIG. 28A-28C are lines graphs showing cumulative fold expansion up to 4 days after viral delivery by the SOLUPORE™ process or static transduction (n=18). Impact on expansion due to viral delivery method (FIG. 28A), MOI of LV (FIG. 28B) and changes made between Run 1/2 and Run 3/4 (FIG. 28C) are shown. Statistical analysis calculated using a three-way ANOVA (* p<0.05, ** p<0.01, *** p<0.001).

FIGS. 29A-29F are line graphs showing GFP expression (FIG. 29A, FIG. 29C, FIG. 29E) and median fluorescence intensity (FIG. 29B, FIG. 29D, FIG. 29F) up to 4 days after viral delivery by the SOLUPORE™ process or static transduction (n=3 technical replicates). Impact on GFP expression and MFI due to viral delivery method (FIG. 29A, FIG. 29B), MOI of LV (FIG. 29C, FIG. 29D) and changes made between Run 1/2 and Run 3/4 (FIG. 29E, FIG. 29F) are shown. Statistical analysis calculated using a three-way ANOVA (* p<0.05, ** p<0.01, ***p<0.001).

FIGS. 30A-30F are bar graphs showing the fold expansion for Run 1 (FIG. 30A), Run 2 (FIG. 30B), Run 3 (FIG. 30C) and Run 4 (FIG. 30D) up to 4 days after viral delivery by the SOLUPORE™ process or static transduction (n=3 technical replicates). Impact on expansion due to MOI of LV for Run 3 (FIG. 30E) and Run 4 (FIG. 30F) are shown. Statistical analysis calculated using paired two-tailed t-test (* p<0.05, ** p<0.01, *** p<0.001).

FIGS. 31A and 31B are bar graphs showing fold expansion up to 4 days after viral delivery by the SOLUPORE™ process or static transduction (n=18). Impact on expansion due to viral delivery method (FIG. 31A) and MOI of LV (FIG. 31B) are shown. Statistical analysis calculated using paired two-tailed t-test (* p<0.05, ** p<0.01, *** p<0.001).

FIG. 32 is a bar graph showing fold expansion up to 4 days after viral delivery by the SOLUPORE™ process or static transduction (n=6 per condition). Statistical analysis calculated using a one-way ANOVA (* p<0.05, ** p<0.01, *** p<0.001).

FIG. 33 is a bar graph showing viability up to 4 days after viral delivery by the SOLUPORE™ process or static transduction (n=6 per condition). Statistical analysis calculated using a one-way ANOVA (* p<0.05, ** p<0.01, *** p<0.001).

FIG. 34 is an image of a Falcon tube during isolation of PBMCs.

FIG. 35 are images showing assembly of the system chamber FIG. 36 is an image of an assembled system base in stand.

FIG. 37 is an image showing wetting of the drain disc.

FIG. 38 is an image show an example of a bad and good layout of filter membrane.

FIG. 39 is an image depicting the Elveflow module and reservoir holder with fluidics.

FIG. 40 is an image showing a controller unit for the SOLUPORE™ method.

FIG. 41 is an image showing calibration cup in system chamber.

FIG. 42 is an image showing a manual pressure regulator to establish airflow through Showerhead.

FIG. 43 is an image showing a control unit to sample line connection.

FIG. 44 is an image showing rotation of the chamber to facilitate rinses and cleaning of surfaces within the chamber.

FIG. 45 is an image of the overview of the system chamber components.

FIG. 46 is an image of the overview of the system lid components.

FIG. 47 is an image showing an assembled system.

FIG. 48 is a an image of a map of the LV expression plasmid with eGFP.

FIG. 49 is a bar graph showing the estimated copies of GFP per cell (GFP %) based on WPRE per 2 albumin for Experiment 1.

FIG. 50 is a bar graph showing the estimated copies of GFP per cell (GFP %) based on WPRE per 2 albumin for Experiment 2.

FIG. 51 is a graph depicting the droplet size distribution (x-axis) versus frequency (y-axis).

FIG. 52 is a graph showing the temperature (x-axis) and dynamic viscosity (y-axis) of various liquids; graph reproduced from Engineering ToolBoox (2008); Dynamic Viscosity of Common Liquids, incorporated herein by reference in its entirety.

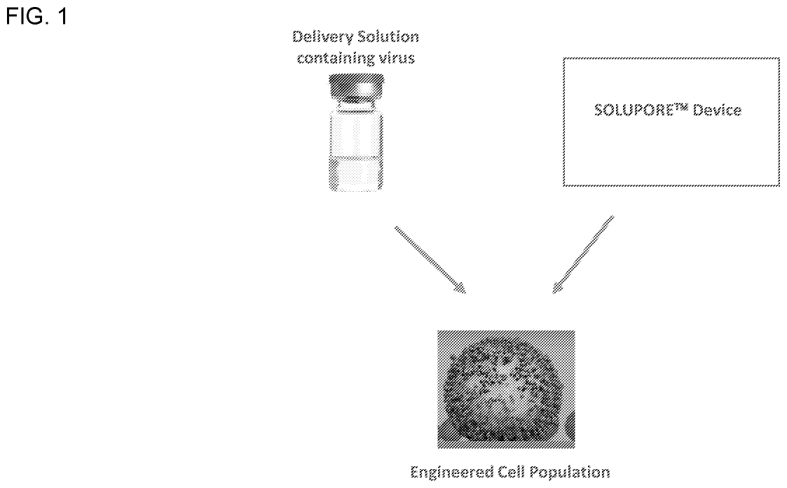

### DETAILED DESCRIPTION

Viruses are widely used as effective gene-delivery vehicles. Transduction is the process by which foreign DNA is introduced into a cell by a virus or viral vector.

Transduction is a common tool used by molecular biologists to stably introduce a foreign gene into a host cell, e.g., mammalian, genome. Optimization of the conditions for transduction is of a high importance for a variety of research applications and clinical applications. For hard to transfect cells, lentivirus transduction offers a high efficiency solution for attaining good expression levels. Efficiency, time, production costs, and cell viability remain challenges in the field.

### Viral Delivery to a Population of Cells Using the SOLUPORE™ Process

The SOLUPORE™ process enables the delivery of a wide range of cargo to adherent and suspension cells in vitro and ex vivo. To date, these cargos have consisted of molecules such as nucleic acids and proteins and particles such as Qdots. The data herein show that the SOLUPORE™ process can be used to deliver a non-bacterial microorganism such as a virus, (e.g., a lentivirus) to T cells with efficiency higher than standard control transduction.

### Viruses as Vectors

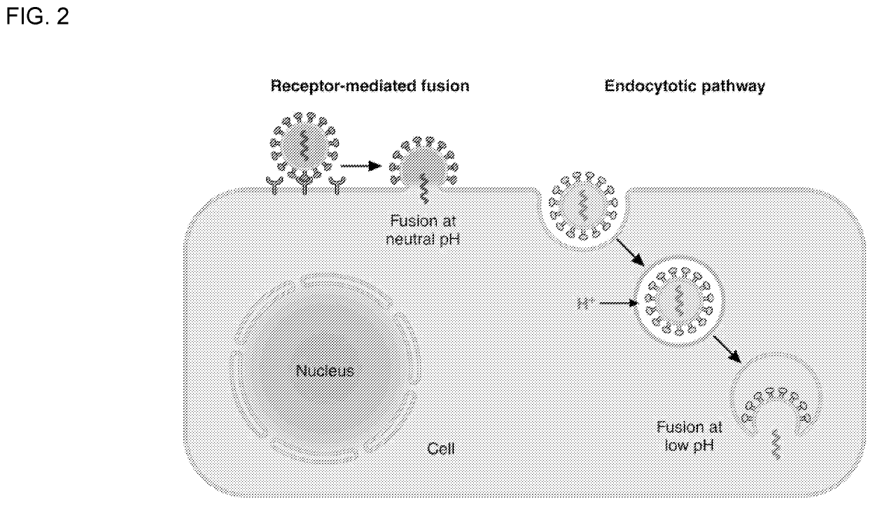

Viruses are used as vectors for delivery of nucleic acids to cells, because they can naturally infect human cells. Prior to entry, a virus must attach to a host cell. Attachment is achieved when specific proteins on the viral capsid or viral envelope bind to specific receptor proteins on the cell membrane of the target cell. Depending on the type of virus, entry into the cell can occur in different ways. Viruses with a viral envelope can enter the cell by membrane fusion where the cell membrane is punctured and made to further connect with the unfolding viral envelope. Viruses with no viral envelope can enter by endocytosis (FIG. 2). Other viruses such as bacteriophages attach to the cell surface, and only the viral genome is injected into the host cells.

### Immune Cell Engineering

Different types of viruses have different features which need to be considered if they are being used to transduce cells ex vivo for clinical applications. The main features are: immunogenicity; target cell type; payload capacity; ability to transduce non-dividing versus dividing cells; transient versus stable genome integration (Table 5).

### Summary of Viruses Used for Gene Delivery Applications.

| Virus | Description | Advantages | Disadvantages |
|---|---|---|---|
| Adenoviruses (AdVs) | non-enveloped dsDNA- | efficient in a broad range of | high immunogenicity; |
| | virus able to carry ≤8 kbp | host cells | transient expression |
| | DNA | | |
| Adeno-associated viruses | non-enveloped recombinant | efficient in a broad range of | small carrying capacity |
| (AAVs) | ssDNA-virus with a small | host cells; non- | |
| | carrying capacity (≤4 kbp) | inflammatory/pathogenic | |
| Retroviruses | enveloped ssRNA-carrying | long-term expression | limited tropism to dividing |
| | virus with ≤8 kbp RNA | | cells; random integration |
| | capacity | | |
| Lentiviruses | enveloped ssRNA-carrying | efficient in a broad range of | potential oncogenic |
| | virus with ≤8 kbp RNA | host cells; long-term | responses |
| | capacity | expression | |
| Herpes simplex viruses | enveloped dsDNA-virus | efficient in a broad range of | potential inflammatory |
| (HSV)-1 large packing | with >30 kbp carrying | host cells | responses; transient |
| capacity; | capacity | | expression |

Pharmaceutics 2020, 12, 183, incorporated herein by reference in its entirety

For immune cell therapy applications such as delivery of chimeric antigen receptor (CAR) constructs for generation of CAR-T and CAR-NK cells, gammaretroviruses and lentiviruses are typically used because they are capable of transducing immune cells and because they result in stable integration into the genome. For example, the first two approved CAR-T cell products, Kymriah and Yescarta, were engineered using lentivirus and gammaretrovirus vectors respectively (Poorebrahim M et al. Crit Rev Clin Lab Sci. 2019 September; 56(6):393-419). As with most viral vectors, these vectors were modified in ways that rendered the viruses replication-incompetent and improved cell targeting efficiencies.

For other immune cell engineering applications such as gene editing, AAV vectors are widely used. While wild type AAVs can stably integrate into chromosome 19, AAV-based gene therapy vectors have been modified to prevent integration and instead form episomal concatemers in the host cell nucleus. In non-dividing cells, these concatemers remain intact for the life of the host cell. In dividing cells, AAV DNA is lost through cell division, since the episomal DNA is not replicated along with the host cell DNA. So AAV vectors are often used to deliver editing systems such as the CRISPR/Cas9 system or donor template DNA for gene editing. In these cases, while the gene edit is permanent, it can be desirable to only transiently express the gene editing tools in order to limit non-specific off-target gene editing that can occur if the tools are present in the cells for extended time periods.

### Lentiviruses

Gammaretroviruses and lentiviruses are subtypes of retroviruses, which contain an RNA genome that is converted to DNA in the transduced cell by a virally encoded enzyme called reverse transcriptase. For retroviruses, entry into the cell is followed by a process of uncoating whereby several viral proteins dissociate from the viral core. The viral RNA is reverse transcribed to double stranded DNA. Viral proteins then complex with the proviral DNA to bring about nuclear import and integration into the host genome. The process of integration is assisted by crucial viral proteins, such as integrase, and endogenous host cell transcription factors.

Lentiviral vectors derived from the human immunodeficiency virus (HIV-1) have become major tools for gene delivery into mammalian cells and replication-deficient recombinant lentiviruses are widely used in research and clinical applications. While the modified lentivirus is still able to infect cells, the essential genes for producing new viral particles are no longer present. Lentiviral vectors are regarded as attractive gene-delivery vehicles for several reasons: they offer long term gene expression via stable vector integration into host genome; they are capable of infecting both dividing and non-dividing cells; they are capable of infecting a broad range of cells including important target cell types for gene and cell therapies; they lack immunogenic viral proteins after vector transduction; they can deliver complex genetic elements such as intron-containing sequences; they are a relatively easy system for vector manipulation and production. Lentiviral vectors have a safer integration site profile than gammaretroviral vectors and are commonly used in clinical trials of CAR T cell therapies (McGarrity G. J. et al. J. Gene Med 2013; 15:78-82). Third-generation lentiviral vectors incorporate key safety features, further enhancing safety (Kim V. N. et al. J. Virol. 1998; 72:811-816 and Dull T. et al. J. Virol. 1998; 72:8463-8471).

The range of cell types that are transducible with retroviruses has been broadened by pseudotyping retroviruses with the envelope glycoprotein G of the vesicular stomatitis virus (VSV-G). VSV-G binds the ubiquitous membrane component phosphatidylserine, which enables the VSV-G pseudotyped virus to attach and transduce a much wider range of cells. Currently, most lentiviral vectors are pseudotyped with VSV-G to enable robust transduction into many cell types including neurons, lymphocytes, and macrophages.

### Quiescent Cells

Following their isolation from either healthy donors or patients, cells are activated and subsequently transduced by lentiviral vectors (LVs), the majority of which are pseudotyped with the glycoprotein G of vesicular stomatitis virus (VSV). The modified lymphocytes are then expanded and either used in functional in vitro assays or used for in vivo applications. Stable and efficient transduction of B cells by LVs is very difficult to achieve. Quiescent B cells are restrictive to transduction by conventional VSV G-pseudotyped LVs (VSV-LVs) due to a lack of low-density lipoprotein receptor (LDLR) expression and they must be activated prior to transduction. Efficient activation and culture of primary human B lymphocytes is complex, as it involves carefully titrated activating stimuli in combination with cytokines followed by co-cultivation with feeder cells. Even under optimal activation and culture conditions, transduction efficiencies with VSV-LVs are notoriously low. The combination of all these difficulties may serve as an explanation for the much lower number of clinical trials involving engineered B cells as compared to T cells.

In comparison, T lymphocyte manipulation by lentiviral transduction is easier to achieve. Still, the cells have to be activated prior to transduction with conventional VSV-LV, because, like B cells, they are otherwise not susceptible for transduction, again due to a lack of LDLR expression (X Geng, et al. Gene Therapy v. 21, pages 444-449(2014)). Mainly due to the paramount success of CAR T cell therapy, the protocols for T cell isolation, activation, lentiviral transduction, and expansion have been extensively improved in recent years. Current state-of-the-art T cell activation relies on stimulation of the TCR activation pathway via CD3- and CD28-specific antibodies in combination with cytokines such as IL-7 and IL-15.

The need for activation of lymphocytes prior to transduction with conventional LVs has disadvantages. It adds to the complexity of the overall procedure increasing duration and costs of the manufacturing process. In addition, the stimuli applied for activation in combination with the prolonged ex vivo culture likely changes the cells, which can negatively impact on the quality of the final product. As a result, naive cells could differentiate into less preferential phenotypes that exhibit a higher degree of exhaustion, lower proliferative capacity, shorter in vivo persistence, and less functionality. This can have very important implications for therapeutic success. For instance, it has been shown that a central memory (CD45RO+/CD45RA+/CD62L+) or stem cell memory (CD45RO+/CD45RA/CD62L+) phenotype is beneficial for T cell persistence and function in vivo. In this regard, a positive correlation of a CAR T cell central memory phenotype and a positive clinical response has been observed in several clinical studies, and, consequently, the infusion of purified central memory CAR T cells is now being considered. Likewise, a central memory phenotype leads to functionally superior TCR-modified T cells. Minimal manipulation of lymphocytes during genetic modification is thus of significant clinical relevance.

### Spinoculation

Centrifugal inoculation, or spinoculation, is widely used in virology research to enhance viral infection. The procedure involves centrifuging a mixture of virus and target cells at high speed for a prolonged period for example 800×g for 30 minutes at 32° C. It was thought that the method enhances transduction rates by concentrating virus at the cell membrane. However, it has been shown that spinoculation triggers dynamic actin and cofilin activity, probably resulting from cellular responses to centrifugal stress (Jia Guo, et al. J. Virology October 2011, p. 9824-9833). This actin activity also leads to the upregulation of cell membrane receptors that may enhance viral binding and entry. It has been suggested that spin-mediated enhancement cannot be explained simply by a virus-concentrating effect; rather, it is coupled with spin-induced cytoskeletal dynamics that promote receptor mobilization, viral entry, and postentry processes. Therefore, spinoculation may affect the biology of the target cell in unknown ways or in ways that are undesirable.

### Limitations of Viral Vectors

While viruses have been useful for genetic engineering of cells as described above, there are limitations to their utility.

High-cost manufacturing processes of Chimeric Antigen Receptor (CAR) T-cells therapies are prohibitively expensive. Due to the cost of virus, transduction is a major cost driver in CAR T-cell manufacturing. Several bioprocessing parameters have been identified as potentially playing a role in transduction efficiency, such as the physical proximity of lentivirus particles to T cells. This proximity could be manipulated through the number of cells and virus particles in the suspension; the periods of agitation to encourage homogeneity; and the surface-to-volume ratio in the transduction vessel. However, limited research has been performed on identifying and optimizing critical process parameters of transduction. During the SOLUPORE™ process, a small volume of delivery solution is applied directly onto exposed target cells. In this way, the cargo is brought directly in contact with the cells in a gentle manner. Delivering viruses to cells in this way leads to a concentration of material at the cell membrane. This process enhances viral attachment to the cell membrane and enhances the rate of entry into the cell making the process more efficient. In turn, small doses of virus are used and costs are reduced.

Because the SOLUPORE™ process is a gentle method of concentrating virus at the cell membrane, it has significant advantages to existing concentration methods such as spinoculation which can affect cell structure. Furthermore, unlike spinoculation, the SOLUPORE™ process is designed to be compatible with cell therapy manufacturing processes.

The concentration of viruses at the cell membrane also compensates for the low levels of expression of viral receptors on certain cell types such as unactivated T cells and thus enhances transduction efficiencies in these cells.

Efficiency of lentiviral vector transduction of unactivated T cells and B cells is typically very low. It is highly desirable to improve these efficiencies, and the SOLUPORE™ process provides a solution to tis problem with high efficiency rates coupled with conditions that are compatible with preservation of cell viability and function.

Viruses are only capable of delivering nucleic acid which means they are restricted in the type of cargo that they can deliver. If viruses could be co-delivered with other types of cargo, it could enhance the utility of viruses in the engineering on next-generation cell therapy products. However, there is currently no method that has been demonstrated to co-deliver viruses with other types of cargo. Again, the SOLUPORE™ process described herein provides a solution to this problem by permitting efficient delivery of numerous different cargo types sequentially or simultaneously. The following materials and methods were used to generate date described herein.

### LV-GFP Vector

The LV-GFP vector used here carries the vesicular stomatitis virus-G (VSV-G) envelope protein, known to target a wide variety of cell types.

### Stability of LV-GFP in Delivery Solution

The stability of LV-GFP in delivery solution was evaluated over an hour by assessing precipitation under a microscope.

### LV-GFP Delivery

Primary human PBMCs were thawed and activated for 3 days with Miltenyi CD3 and CD28 antibodies. After 3 days of activation culture the cells underwent the SOLUPORE™ process or static transduction with LV-GFP (MOI=2.5). Cells were recovered for 72 hours before GFP expression was assessed by flow cytometry.

### Stability of LV-GFP in Delivery Solution

Prior to the invention, the SOLUPORE™ process delivery had not been previously combined with a virus preparation so it was unknown whether there would be solubility problems.

No aggregation or precipitation was observed when the solution was viewed under the microscope during the stability analysis. Furthermore, certain other cargos have exhibited low levels of aggregation when combined with the delivery solution and have caused the Solupore® atomizer to block during spraying. When the delivery solution containing the virus was loaded into the Solupore® atomizer and sprayed, no blocking occurred at any point over the course of the study, further indicating that no aggregation or precipitation occurred.

### Cell Viability, Expansion and GFP Expression Following LV-GFP Delivery

LV-GFP was delivered to T cell cultures by the SOLUPORE™ process and compared with a standard static method of LV transduction.

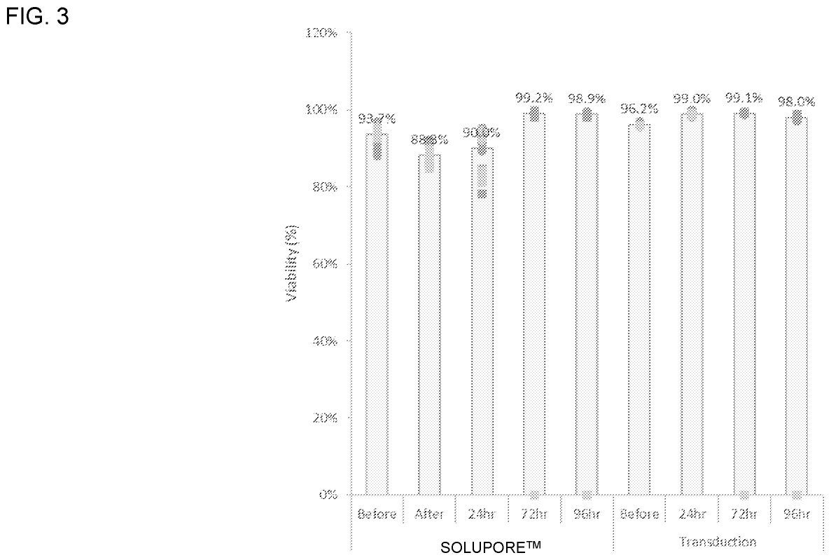

The viability of cells was measured at various timepoints before and after the delivery of virus. At all timepoints, the viability of soluporated cells was comparable to that of control transduced cells (FIG. 3).

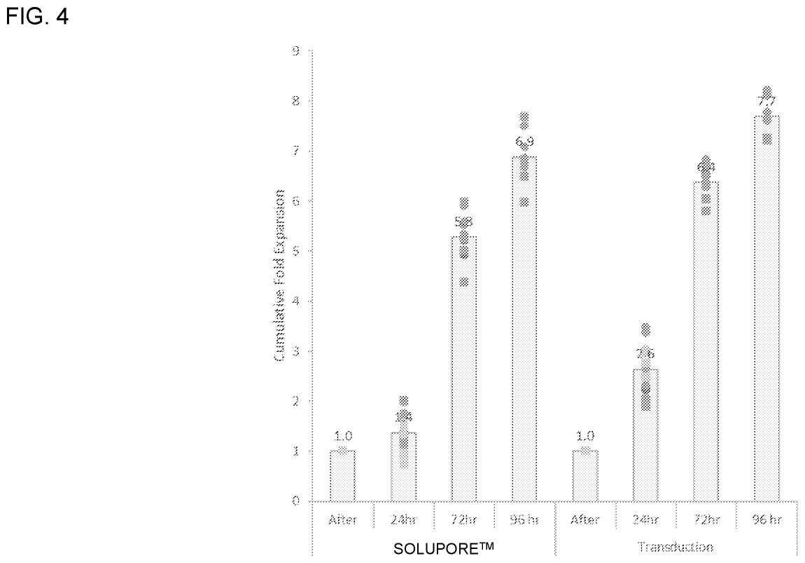

The cumulative fold expansion of the T cells was determined up to 96 hr after delivery of virus. The expansion of soluporated cells was comparable to that of control transduced cells (FIG. 4).

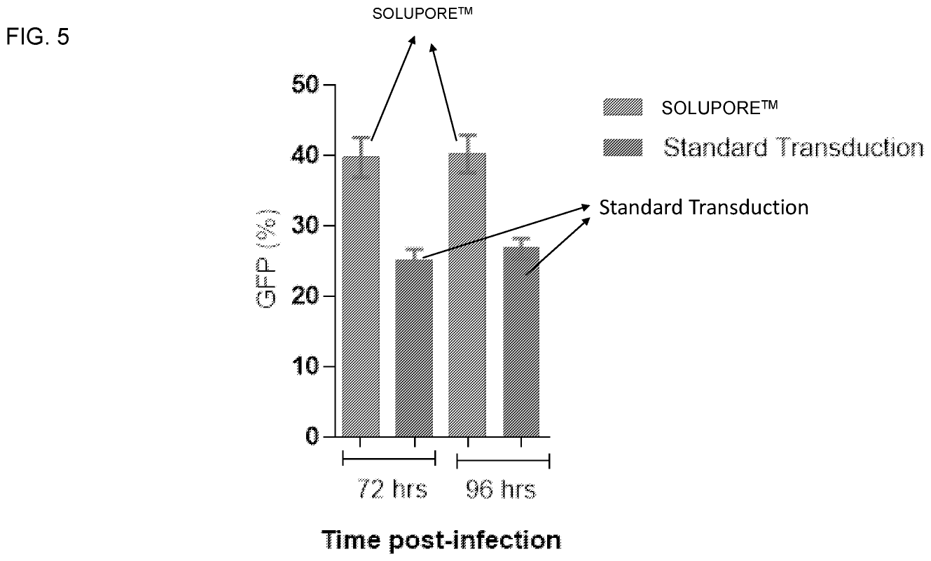

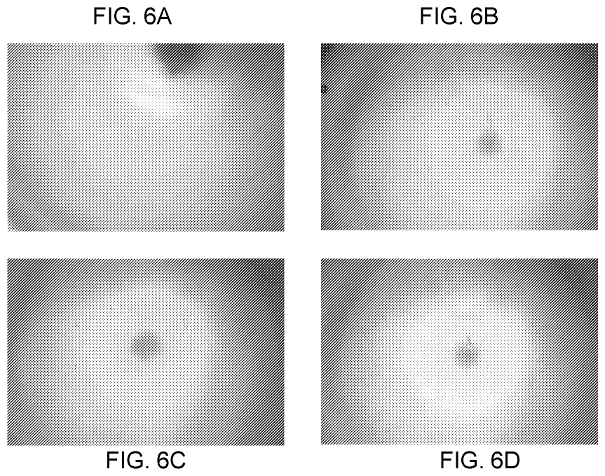

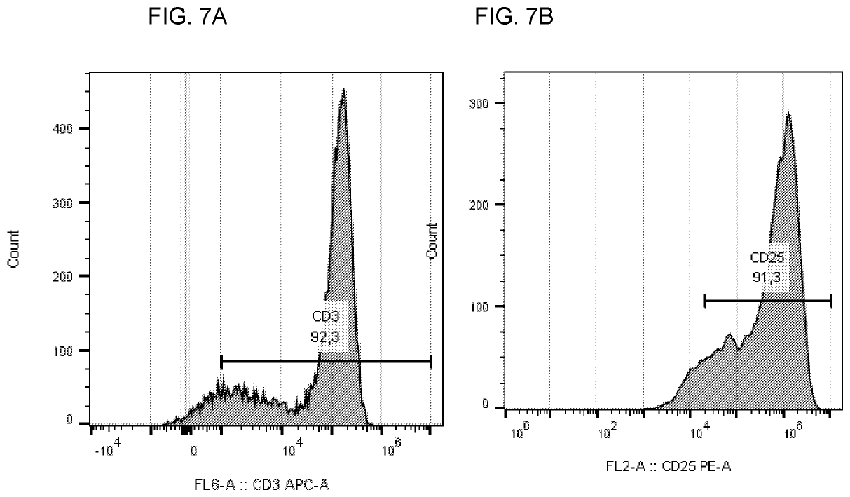

The expression of GFP was measured at day 3 and day 4 post-delivery. GFP expression efficiency was higher in soluporated T cells compared with control transduced cells (FIG. 5). At day 3, efficiency was 39.73±2.83% compared with 25.2±1.48% for soluporated cells and control transduced cells respectively. At day 4, efficiency was 40.27±2.67% compared with 26.83±1.38% for soluporated cells and control transduced cells respectively.

### Efficient Viral Delivery of Virus to a Population of Cells

The data herein demonstrated that the SOLUPORE™ process is compatible with delivery of virus to activated T cells. When mixed with the SOLUPORE™ process delivery solution, no precipitation or aggregation was observed. It was possible to spray the viral solution and the target cells were successfully transduced. The viability and the expansion rate of the soluporated T cells were unaffected.

GFP expression was higher in soluporated T cells compared with control transduced cells indicating that the SOLUPORE™ process enhances viral transduction of T cells.

Together these finding demonstrate that the SOLUPORE™ process is suitable for delivery of viruses to cells and is superior to standard methods.

Because the SOLUPORE™ process is suitable for delivery of virus, it is possible to use soluporaton in cell therapy manufacturing processes that involve viral transduction. Due to the cost of virus, transduction is a major cost driver in CAR T-cell manufacturing. Because the SOLUPORE™ process enhances viral transduction, it is now possible to use less virus to achieve similar levels of transduction efficiency and thus reducing costs.

Different cargos can be delivered simultaneously by the SOLUPORE™ process. The demonstration herein that the SOLUPORE™ process is compatible with viral delivery means that the SOLUPORE™ process can be used to co-deliver virus with other cargos. These other cargos could be other viruses or could be proteins, nucleic acids, small molecules or complexes thereof. The ability to co-deliver cargos means that engineering steps that would otherwise happen in different process steps can be combined into a single process step. This process has major benefits for manufacturing processes including cost, time and labor. In addition, fewer process steps means less handling and risk of contamination as well as simplifying the process. Alternatively, virus is delivered in sequence, before or after other cargos.

The SOLUPORE™ process enables delivery of cargo to unactivated T cells.

Lentiviral vectors have very low transduction efficiency in unactivated T cells. Therefore, the SOLUPORE™ process increases the transduction efficiency of lentivirus in unactivated T cells.

A core feature of the SOLUPORE™ process device is its ability to facilitate changes of medium. When a solution containing cells is transferred into the device, the liquid can be drained away and replaced with different liquids. In this way, liquid handling steps are possible. Such liquid handling steps could include for example wash steps. With viral transduction and other cell manufacturing process, wash steps are often required. The Solupore® device enables integration of such steps into a manufacturing process.

The Solupore® technology is also scalable which means that viral transduction using this method could be carried out at small scale for early and pre-clinical work as well as larger scales for process development and clinical applications.

### Advantages and Surprising Results of the Methods

### How Viruses are Different Compared to Other Cargos

A virus is a microorganism, e.g., submicroscopic infectious agent that replicates only inside the living cells of an organism. Viruses can infect all types of life forms, from animals and plants to microorganisms, including bacteria and archaea. A virus is made up of a core of genetic material, either DNA or RNA, surrounded by a protective coat called a capsid which is made up of protein. Sometimes the capsid is surrounded by an additional spikey coat called the envelope. Viruses are capable of latching onto host cells and getting inside them.

Previously, cargos used with Solupore™ have been either a single class of molecule (such as a protein or a nucleic acid), or mixtures of molecules, or complexes of molecules (such as Cas9 ribonucleoproteins (RNPs)). A virus is a more complex agent in terms of structure and function. Regarding structure, the genetic material is single stranded RNA that is contained within a protein capsid, and so is encapsulated. Lentivirus virions contain 2% nucleic acids, 60% protein and 35% lipid and 3% carbohydrates. It is expected that the structure must remain intact in order for the virus to successfully infect the cell. Regarding function, it is expected that once inside the cell, the virus must remain capable of releasing its genetic material in order to express the transgene. It is also expected that the host cell must remain viable and functional in order to subsequently express the viral transgene.

In terms of size, lentivirus ranges between 80-100 nm in diameter. In contrast, in respect of the components of a typical RNP, SpCas9 protein is ˜7.5 nm hydrodynamic diameter (160 kDa) with a net positive surface charge and sgRNA is 5.5 nm hydrodynamic diameter (˜31 kDa) and is negatively charged (Bioconjug Chem. 2017 Apr. 19; 28(4): 880-884). Therefore an RNP is significantly smaller than a lentivirus viroid. Surprising results and unexpected advantages of viral transduction using the Solupore™ process

The Solupore™ process includes has been used to deliver relatively simple cargo molecules (but not complex cargoes such as a microorganism) into mammalian cells. The process involves a number of steps, and prior the invention, it was unknown whether one, several, and/or all of these steps would be compatible with successful viral infection as outlined above.

1. Mixing the Cargo with the Delivery Solution

It was unknown whether the viral preparation described herein would be compatible with the delivery solution. Occasionally, other cargos have been observed to aggregate, thus hindering the process. Such aggregation causes several problems such as loss of cargo functionality and blocking of the Solupore™ fluidic pathway. Sometimes, the extent of this aggregation is substantial such that it is immediately apparent and an experiment is aborted. However, sometimes aggregation is not detectable during the Solupore™ experiment and only becomes identified when an analytical assay is carried out to test for cargo activity in the target cells. Subsequent trouble shooting has led to detection of partial blockages in the fluidic pathway that were not sufficient to interfere with the completion of an experiment but likely altered the outcome.

For this work, delivery solution compatibility tests were carried out and surprisingly, no aggregation was visible under the microscope. In addition, no blocking of the device was observed. In addition, a successful outcome, namely expression of the GFP transgene, was achieved. These finding indicate that the viral preparation was compatible with the delivery solution. This was surprising given the complexity of the virus agent. Furthermore, the absence of observable blocking of the fluidic pathway indicates that no aggregation occurred within the tubing or while the solution was being transferred through the system. Again this was surprising given the complexity and size of the virus agent.

2. Forming Droplets of Certain Size and Velocity

It was unknown whether the viral preparation described herein would be compatible with the aerosolisation process. During the process, the delivery solution is broken up into droplets using a bespoke atomizer that has not been previously used with viruses. However, the results described herein indicate that the viral preparation was compatible.

3. Allowing Those Droplets to Travel Specific Distances

These droplets (e.g., the delivery solution which is broken up into droplets using a bespoke atomizer) are then driven towards the target cells across a distance of 75 mm. During transit, as with a typical aerosoliation process, the droplets will be subject to evaporation and condensation. It was unknown before this work whether this would adversely affect the ability of the viruses to (1) remain active, (2) enter the cells and (3) go on to express the transgene. The results indicate that the viral preparation was compatible with all three steps of the process.

4. Allowing the Droplets to Land on Target Cells

The droplets landed on the target cells with approximately 17 g force and it was unknown whether this was compatible with retaining integrity and functionality of the virus particles. The results indicated that the viral preparation was compatible with this process.

5. Incubating the Cells with the Applied Solution

Once applied onto the cells, the delivery solution is incubated with the cells for 30 seconds. Because only a very small volume is applied to a relatively large area, 50-100 microlitres to 2827.43 mm², it is expected that there will be evaporation and drying and it was unknown whether this would adversely affect the ability of the virus to infect the cells. The results indicate that the viral preparation was compatible with this process.

6. Applying a Second Solution

After the incubation step, a second solution is applied onto the cells. It was unknown whether this would adversely affect the ability of the virus to infect the cells. It could have resulted in dilution of extracellular virus or adversely affected the virus in some other way. The results indicate that the viral preparation was compatible with this process.

7. Removing the Cells from the SOLUPORE™ Process Chamber

In order to remove the cells from the chamber and place them in culture, it is necessary to flush the chamber several times and to agitate the cell suspension. It was not known whether this was likely to interfere with the viral infection process in some way for example by damaging the cells such that they could not successfully express the viral transgene. The results indicate that the viral preparation was compatible with this process.

8. Culturing the Cells for Several Days

During viral infection, virus-derived cytosolic nucleic acids are recognized by host intracellular specific sensors. The efficacy of this recognition system is crucial for triggering innate host defenses, which then stimulate more specific adaptive immune responses against the virus (Lee, H., Chathuranga, K. & Lee, J. Intracellular sensing of viral genomes and viral evasion. Exp Mol Med 51, 1-13 (2019) ). Before this study, it was not known whether the above Solupore™ process would affect the cells in some way that would cause them to be more sensitive than normal to the presence of virus. If this occurred it was possible that the viability of the cells could be compromised and that they would not survive for the duration of the 4 day post-infection culture period. Alternatively, it was possible that they would survive but their health would be compromised such that they could not express the transgene. Surprisingly, the cells into which the virus was delivered experienced approximately 90% viability.

### Viral Infection Process and Droplet Properties

The atomisation of lentivirus within the transfection chamber is a distinct process from the SOLUPORE™ process. As described herein, the cargo delivered to the population of cells is a virus (e.g., a lentivirus), that is biologically active and viable.

Typical titres of lentivirus range from 106 to 10⁷ transducing units per milliliter (TU/ml) and the consistency of lentivirus at these concentrations is highly, dynamically viscous relative to water/ethanol mixtures. For illustration, (Table 1) the dynamic viscosity of water at room temperature is close to 1 mPa s, the dynamic viscosity of ethanol is close to 0.1 mPa s, the dynamic viscosity of olive oil is close to 60 0.1 mPa s and the dynamic viscosity of castor oil is close to 600 0.1 mPa s. The dynamic viscosity of lentivirus was reported by Trarm Reginald, PhD Thesis, Georgia Tech (2016), as 6913 mPas, being close to being a log more viscous than castor oil. Sterile filtered 1% bovine serum albumin (BSA) has been found by some to decrease molecular interactions that might lead the virus particles to “stick” to the injection apparatus. (Jasnow A. et al. Methods Mol Biol. “Construction of Cell-Type Specific Promoter Lentiviruses for Optically Guiding Electrophysiological Recordings and for Targeted Gene Delivery” 2009; 515: 199-213, incorporated herein by reference in its entirety).

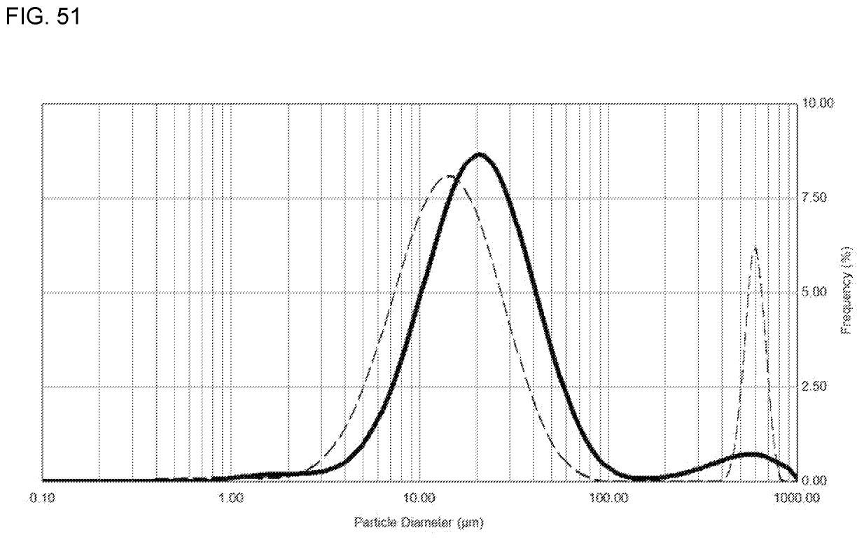

Dynamic Viscosity is an important factor in atomisation. Experimental studies on atomization in an internal-mixing twin-fluid atomizer, such as that used in the SOLUPORE™ process, over a wide range of liquid viscosity, gas supply pressure and Gas to Liquid mass Ratio (GLR) have been performed. See, e.g., Li, Z. et al. “Effect of liquid viscosity on atomization in an internal-mixing twin-fluid atomizer” Fuel vol. 103; January 2013 pages 486-494, incorporated herein by reference in its entirety. Among all test conditions, the finest sprays were obtained at an axial distance of 150 mm. However, droplet size distributions notably changed when viscosity increased to 120 mPa s. The higher viscosity droplets produced larger droplets (e.g., 1 to 2 logs larger than the current droplets produced by the SOLUPORE™ processed measured droplet size distribution, FIG. 51). The larger droplets represented a large proportion of the droplet population (distribution), and the decay of droplet velocities along the spray axis was stronger at a larger viscosity.

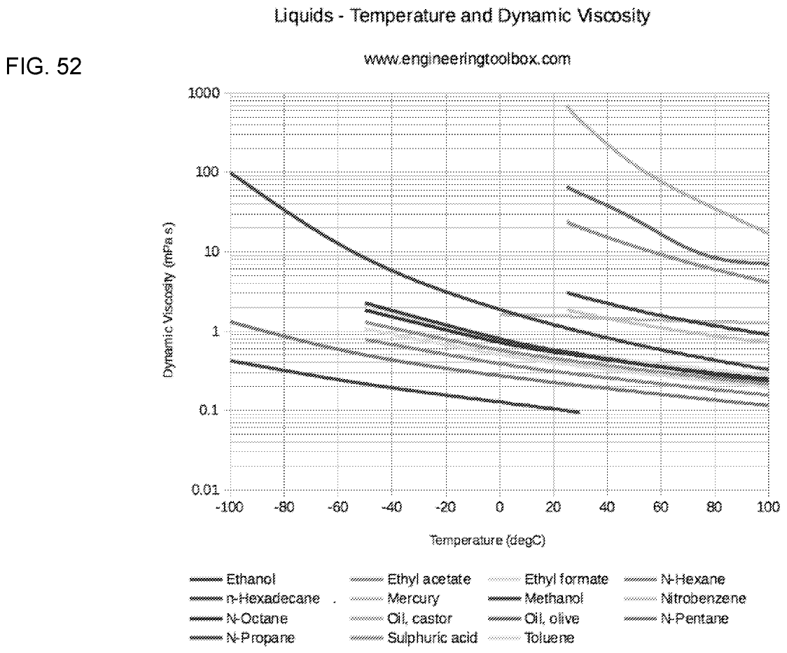

A table showing the dynamic viscosities of common liquids is shown below (and graph provided at FIG. 52).

Absolute or dynamic viscosities for some common liquids at temperature 300 K are indicated below:

| | Absolute Viscosity | | |
|---|---|---|---|
| Fluid | (N s/m², Pa s) | (centipoise, cP) | (10⁻⁴ lb/s ft) |
| Acetic acid | 0.001155 | 1.155 | 7.76 |
| Acetone | 0.000316 | 0.316 | 2.12 |
| Alcohol, ethyl | 0.001095 | 1.095 | 7.36 |
| (ethanol) | | | |
| Alcohol, methyl | 0.00056 | 0.56 | 3.76 |
| (methanol) | | | |
| Alcohol, propyl | 0.00192 | 1.92 | 12.9 |
| Benzene | 0.000601 | 0.601 | 4.04 |
| Blood | 0.003-0.004 | | |
| Bromine | 0.00095 | 0.95 | 6.38 |
| Carbon Disulfide | 0.00036 | 0.36 | 2.42 |
| Carbon Tetrachloride | 0.00091 | 0.91 | 6.11 |
| Castor Oil | 0.650 | 650 | |
| Chloroform | 0.00053 | 0.53 | 3.56 |
| Decane | 0.000859 | 0.859 | 5.77 |
| Dodecane | 0.00134 | 1.374 | 9.23 |
| Ether | 0.000223 | 0.223 | 1.50 |
| Ethylene Glycol | 0.0162 | 16.2 | 109 |
| Trichlorofluoromethane | 0.00042 | 0.42 | 2.82 |
| refrigerant R-11 | | | |
| Glycerine | 0.950 | 950 | 6380 |
| Heptane | 0.000376 | 0.376 | 2.53 |
| Hexane | 0.000297 | 0.297 | 2.00 |
| Kerosene | 0.00164 | 1.64 | 11.0 |
| Linseed Oil | 0.0331 | 33.1 | 222 |
| Mercury | 0.0015 | 1.53 | 10.3 |
| Milk | 0.003 | | |
| Octane | 0.00051 | 0.51 | 3.43 |
| Phenol | 0.0080 | 8.0 | 54 |
| Propane | 0.00011 | 0.11 | 0.74 |
| Propylene | 0.00009 | 0.09 | 0.60 |
| Propylene glycol | 0.042 | 42 | |
| Toluene | 0.000550 | 0.550 | 3.70 |
| Turpentine | 0.001375 | 1.375 | 9.24 |
| Water, Fresh | 0.00089 | 0.89 | 6.0 |

It can be concluded from that the atomisation of lentivirus within the SOLUPORE™ process produced larger, slower moving droplets and the experience of the layer of cells beneath, are different form the previously described SOLUPORE™ process. Consequently, this atomisation process resulted in transfection levels of close to 30%, a surprising and unexpected observation.

### Viral Infection Process

The dynamic viscosity of water is close to 1 mPa s (milli Pascale seconds). The dynamic viscosity of ethanol/water mixes is also close to 1 mPa s. The dynamic viscosity of an aqueous solution that can include an ethanol concentration of 5 to 30%. The aqueous solution can include one or more of 75 to 98% H₂O, 2 to 45% ethanol, 6 to 91 mM sucrose, 2 to 35 mM KCl, 2 to 35 mM ammonium acetate, and 1 to 14 mM (4-(2-hydroxyethyl)-1-piperazineethanesulfonic acid) (HEPES) has a viscosity is the region of 2 mPa s.

The dynamic viscosity of lentivirus at titres 10\hat{ }7 to 10\hat{ }8 TU/mL is close to 6913 mPa s. As the viscosity of a fluid increases, at a given spray pressure, for example 1.7 bar, it will tend to form larger droplets when sprayed. Sprays consisting of smaller droplets have a much larger surface area per volume than those made up of larger droplets. Moreover, the droplets have a lower surface tension than water, and thus the droplets get even larger. In turn, the cells experience an entirely different process. As such finer sprays are better able to spread out on their target surface. This effect is relatively small for fluids with viscosities below 10 mPa s but becomes more pronounced with higher dynamic viscosities. Fluids with higher dynamic viscosities than water or water/ethanol mixes will have higher mean droplet sizes for any given flow rate and pressure. The interplay between the mechanical properties of fluids can be calculated by the generally accepted formula:

$${D}_{f} = {D}_{w} {V}_{f}^{0.2} \qquad Equation [ 1 ]$$

Where D_{f}=modified droplet size for the fluid in question
D_{w}=Droplet size calculated for water
V_{f}=the viscosity of the fluid (viscosity in mPa s; water=1.0 mPA s, lentivirus is 6913 mPa s)

It can be calculated from Equation [1] that Lentivirus droplets (e.g., droplets including a volume of aqueous solution including a virus, an ethanol concentration of 5 to 30% and one or more of 75 to 98% H₂O, 2 to 45% ethanol, 6 to 91 mM sucrose, 2 to 35 mM KCl, 2 to 35 mM ammonium acetate, and 1 to 14 mM (4-(2-hydroxyethyl)-1-piperazineethanesulfonic acid) (HEPES)) sprayed under the same pressure and flow conditions as water/ethanol mixes will have droplet sizes close to 5.9 times larger than the water/ethanol droplets. As described in International Application WO 2016/065341 (incorporated herein by reference in its entirety), droplets in the size range of 30 μm to 100 μm and 50 μm to 80 μm in diameter were described. Generally, in the methods described herein if the aqueous solution being sprayed includes a virus (e.g., a lentivirus), the droplet size range is about 150 μm to 600 μm in diameter, or about 177 μm to 590 μm in diameter. In other examples, the droplet diameter size is 200 μm to 600 μm, or about 300 μm to 600 μm, or about 400 μm to 600 μm, or about 500 μm to 600 μm. In other examples, the droplet size of the invention herein may be larger than 600 μm, for example about 600 μm to 1000 μm in diameter, or about 600 μm to 900 μm, or about 600 μm to 800 μm, or about 600 μm to 700 μm in diameter. In some examples droplet size may be characterized by a diameter of up to 1000 μm, e.g., 150 μm to 1000 μm.

These droplets are much larger than anticipated or described in WO 2016/065341, which “A portion of the colloidal droplets produced can be too large for a given intracellular delivery application. Because a portion of the colloidal droplets produced are too large, cell death may occur notwithstanding the production of appropriately sized colloidal droplets.” See WO 2016/065341 at ¶ [0172]. Accordingly, it was unexpected and surprising that the cells (e.g., cells contacted with an aqueous solution including a virus) tolerated such a different process as compared to the SOLUPRE™ process described in WO 2016/065341, and the cells were infected with lentivirus and viable.

### Droplet Size Properties

The larger diameter droplets of the invention described herein have a larger volume and weight, travel more slowly and impact the cell layer with greater force. For example the volume of a droplet increases by a factor of close to 206.8 when the diameter increases by a factor of 5.9. Thus, the fluid mechanics of this system are distinct from those described in See WO 2016/065341 and constitute a new viral infection process.

### Delivery of Cargo to Cells

Difficulty in transfecting molecules into cells has plagued research and therapeutic, e.g., cell therapy, gene therapy, genetic alteration, for decades. The invention provides a solution to such problems for complex cargo entities such as viruses. Generally, the method for delivering a payload across a plasma membrane of a cell comprises providing a population of cells and contacting the population of cells with a volume of aqueous solution, the aqueous solution including the payload and an alcohol at greater than 2 percent concentration, wherein the volume is a function of: (i) exposed surface area of the population of cells; or (ii) a number of cells in the population of cells, and wherein contacting the population of cells with the volume of aqueous solution is performed by gas propelling the aqueous solution to form a spray.

A reason for the difficulty in transfecting certain types of cells may be that non-adherent cells lack cell surface heparan sulfate proteoglycans, molecules are responsible for adhesion of cells to the extra-cellular matrix. Transfection methods such as electroporation and/or nucleofections have drawbacks in that they compromise the viability of cells, the ability of the cells to resume proliferation after treatment, and the function of the cells, e.g., immune activity of lymphocytes. The transfection/transduction compositions and methods described herein do not have such drawbacks and therefore are characterized as having significant advantages over earlier methods of introducing cargo molecules into mammalian cells, e.g., difficult-to-transfect non-adherent/suspension cells.

The invention is based on the surprising discovery that compounds or mixtures of compounds (compositions) are delivered into the cytoplasm of eukaryotic cells by contacting the cells with a solution containing a virus and an agent that reversibly permeates or dissolves a cell membrane. Preferably, the solution is delivered to the cells in the form of a spray, e.g., aqueous particles. (see, e.g., PCT/US2015/057247 and PCT/IB2016/001895, hereby incorporated in their entirety by reference). For example, the cells are coated with the spray but not soaked or submersed in the delivery compound-containing solution. Exemplary agents that permeate or dissolve a eukaryotic cell membrane include alcohols and detergents such as ethanol and Triton X-100, respectively. Other exemplary detergents, e.g., surfactants include polysorbate 20 (e.g., Tween 20), 3-[(3-cholamidopropyl)dimethylammonio]-1-propanesulfonate (CHAPS), 3-[(3-cholamidopropyl)dimethylammonio]-2-hydroxy-1-propanesulfonate (CHAPSO), sodium dodecyl sulfate (SDS), and octyl glucoside.

An example of conditions to achieve a coating of a population of coated cells include delivery of a fine particle spray, e.g., the conditions exclude dropping or pipetting a bolus volume of solution on the cells such that a substantial population of the cells are soaked or submerged by the volume of fluid. Thus, the mist or spray comprises a ratio of volume of fluid to cell volume. Alternatively, the conditions comprise a ratio of volume of mist or spray to exposed cell area, e.g., area of cell membrane that is exposed when the cells exist as a confluent or substantially confluent layer on a substantially flat surface such as the bottom of a tissue culture vessel, e.g., a well of a tissue culture plate, e.g., a microtiter tissue culture plate or on a filter membrane e.g., on a filter plate, the cells having been exposed by the removal of media.

Advantages of the Presently Claimed Methods

The methods described herein provide a number of advantages over well-known techniques and applications. Exemplary advantages include:

1) ability to transfect the same number of cells with less virus (alternatively, reduces the amount of virus required to infect a population of cells),

2) ability to transfect more cells using the same amount of virus (alternatively, increases the number of transfected cells using the same amount of virus used in current protocols),

3) increased viral uptake,

4) increased cell viability,

5) increased transfection efficiency, and

6) production efficiency (ability to perform multiple steps in a single step or area)

“Cargo” or “payload” are terms used to describe a compound, or composition that is delivered via an aqueous solution across a cell plasma membrane and into the interior of a cell.

In an aspect, delivering a virus across a plasma membrane of a cell includes providing a population of cells and contacting the population of cells with a volume of an aqueous solution. The aqueous solution includes the virus and an alcohol content greater than 2 percent concentration. The volume of the aqueous solution may be a function of exposed surface area of the population of cells, or may be a function of a number of cells in the population of cells.

In another aspect, a composition for delivering a virus across a plasma membrane of a cell includes an aqueous solution including the virus, an alcohol at greater than 2 percent, (e.g., greater than 5 percent) concentration, greater than 46 mM salt, less than 121 mM sugar, and less than 19 mM buffering agent. For example, the alcohol, e.g., ethanol, concentration does not exceed 50%.

In another aspect, a composition for delivering a virus across a plasma membrane of a cell includes an aqueous solution including the virus, greater than 46 mM salt, less than 121 mM sugar, and less than 19 mM buffering agent. For example, the aqueous solution does not include alcohol.

One or more of the following features can be included in any feasible combination.

The volume of solution to be delivered to the cells is a plurality of units, e.g., a spray, e.g., a plurality of droplets on aqueous particles. The volume is described relative to an individual cell or relative to the exposed surface area of a confluent or substantially confluent (e.g., at least 75%, at least 80% confluent, e.g., 85%, 90%, 95%, 97%, 98%, 100%) cell population. For example, the volume can be between 6.0×10⁻⁷ microliter per cell and 7.4×10⁻⁴ microliter per cell. The volume is between 4.9×10⁻⁶ microliter per cell and 2.2×10⁻³ microliter per cell. The volume can be between 9.3×10⁻⁶ microliter per cell and 2.8×10⁻⁵ microliter per cell. The volume can be about 1.9×10⁻⁵ microliters per cell, and about is within 10 percent. The volume is between 6.0×10⁻⁷ microliter per cell and 2.2×10⁻¹ microliter per cell. The volume can be between 2.6×10⁻⁹ microliter per square micrometer of exposed surface area and 1.1×10⁻⁶ microliter per square micrometer of exposed surface area. The volume can be between 5.3×10⁻⁸ microliter per square micrometer of exposed surface area and 1.6×10⁻⁷ microliter per square micrometer of exposed surface area. The volume can be about 1.1×10⁻⁷ microliter per square micrometer of exposed surface area. About can be within 10 percent.

Confluency of cells refers to cells in contact with one another on a surface. For example, it can be expressed as an estimated (or counted) percentage, e.g., 10% confluency means that 10% of the surface, e.g., of a tissue culture vessel, is covered with cells, 100% means that it is entirely covered. For example, adherent cells grow two dimensionally on the surface of a tissue culture well, plate or flask. Non-adherent cells can be spun down, pulled down by a vacuum, or tissue culture medium aspiration off the top of the cell population, or removed by aspiration or vacuum removal from the bottom of the vessel in a filter setting.

Contacting the population of cells with the volume of aqueous solution can be performed by gas propelling the aqueous solution to form a spray. The gas can include nitrogen, ambient air, or an inert gas. The spray can include discrete units of volume ranging in size of greater than 150 μm in diameter.

A total volume of aqueous solution of 20 μl can be delivered in a spray to a cell-occupied area of about 1.9 cm², e.g., one well of a 24-well culture plate. A total volume of aqueous solution of 10 μl is delivered to a cell-occupied area of about 0.95 cm², e.g., one well of a 48-well culture plate. The spray is optionally delivered to larger areas, e.g., the size of a petri dish or even larger area, any size suited to the diameter of the area covered by the spray emitted from the atomizer.

Typically, the aqueous solution includes a virus to be delivered across a cell membrane and into cell, and the second volume is a buffer or culture medium that does not contain the payload. Alternatively, the second volume (buffer or media) can also contain virus. In some examples, the second volume, contains a different type of cargo, e.g., a nucleic acid, protein, or chemical compound (i.e., a non-viral cargo). Alternatively, the first solution contains a non-viral cargo and the second solution contains a viral cargo. The viral and non-viral cargoes may be delivered sequentially as described above or simultaneously, i.e., in the same delivery solution. In some embodiments, the aqueous solution includes a payload and an alcohol, and the second volume does not contain alcohol (and optionally does not contain payload). The population of cells can be in contact with said aqueous solution for 0.1-10 minutes prior to adding a second volume of buffer or culture medium to submerse or suspend said population of cells. The buffer or culture medium can be phosphate buffered saline (PBS). The population of cells can be in contact with the aqueous solution for 2 seconds to 5 minutes prior to adding a second volume of buffer or culture medium to submerse or suspend the population of cells. The population of cells can be in contact with the aqueous solution, e.g., containing the virus, for 30 seconds to 2 minutes prior to adding a second volume of buffer or culture medium, e.g., without the virus, to submerse or suspend the population of cells. The population of cells can be in contact with a spray for about 1-2 minutes prior to adding the second volume of buffer or culture medium to submerse or suspend the population of cells. During the time between spraying of cells and addition of buffer or culture medium, the cells remain hydrated by the layer of moisture from the spray volume.

The aqueous solution can include an ethanol concentration of 2 to 30%, 2 to 40%, Or 2-50%. The aqueous solution can include one or more of 75 to 98% H₂O, 2 to 45% ethanol, 6 to 91 mM sucrose, 2 to 500 mM KCl, 2 to 35 mM ammonium acetate, and 1 to 14 mM (4-(2-hydroxyethyl)-1-piperazineethanesulfonic acid) (HEPES). For example, the delivery solution contains 106 mM KCl and 27% ethanol. In embodiments, the aqueous solution comprises 27% ethanol for the Flexi (e.g., small scale). In embodiments the aqueous solution comprises 12% ethanol in a large scale system.

The population of cells can include adherent cells or non-adherent cells. The adherent cells can include at least one of primary mesenchymal stem cells, fibroblasts, monocytes, macrophages, lung cells, neuronal cells, fibroblasts, human umbilical vein (HUVEC) cells, Chinese hamster ovary (CHO) cells, induced pluripotent stem cells (iPSCs), and human embryonic kidney (HEK) cells or immortalized cells, such as cell lines. In preferred embodiments, the population of cells comprises non-adherent cells, e.g., the % non-adherent cells in the population is at least 50%, 60%, 75%, 80%, 90%, 95%, 98%, 99% or 100% non-adherent cells. Non-adherent cells primary cells as well as immortalized cells (e.g., cells of a cell line). Exemplary non-adherent/suspension cells include primary hematopoietic stem cell (HSC), T cells (e.g., CD3+ cells, CD4+ cells, CD8+ cells), natural killer (NK) cells, cytokine-induced killer (CIK) cells, human cord blood CD34+ cells, B cells, dendritic cells, tumor infiltrating lymphocyte (TILs), or cell lines such as Jurkat T cell line. In other examples, NK cell lines including NK92 and KHYG1 are used.

The population of non-adherent cells can be substantially confluent, such as greater than 75 percent confluent. Confluency of cells refers to cells in contact with one another on a surface. For example, it can be expressed as an estimated (or counted) percentage, e.g., 10% confluency means that 10% of the surface, e.g., of a tissue culture vessel, is covered with cells, 100% means that it is entirely covered. For example, adherent cells grow two dimensionally on the surface of a tissue culture well, plate or flask. Non-adherent cells can be spun down, pulled down by a vacuum, or tissue culture medium aspiration off the top of the cell population, or removed by aspiration or vacuum removal from the bottom of the vessel. Additional removal methods may include gravity, or using magnetic beads plus a magnet The population of cells can form a monolayer of cells.

The alcohol can be selected from methanol, ethanol, isopropyl alcohol, butanol and benzyl alcohol. The salt can be selected from NaCl, KCl, Na₂HPO₄, KH₂PO₄, and C₂H₃O₂NH. In preferred embodiments, the salt is KCl. The sugar can include sucrose. The buffering agent can include 4-2-(hydroxyethyl)-1-piperazineethanesulfonic acid.

The present subject matter relates to a method for delivering viruses across a plasma membrane. The present subject matter finds utility in the field of intra-cellular delivery, and has application in, for example, delivery of molecular biological and pharmacological therapeutic agents to a target site, such as a cell, tissue, or organ. The method of the present subject matter comprises introducing the molecule to an aqueous composition to form a matrix; atomizing the matrix into a spray; and contacting the matrix with a plasma membrane.

This present subject matter relates to a composition for use in delivering viruses across a plasma membrane. The present subject matter finds utility in the field of intra-cellular delivery, and has application in, for example, delivery of molecular biological and pharmacological therapeutic agents to a target site, such as a cell, tissue, or organ. The composition of the present subject matter comprises an alcohol; a salt; a sugar; and/or a buffering agent.

The example methods described herein include an aqueous solution including an alcohol. By the term “an alcohol” is meant a polyatomic organic compound including a hydroxyl (—OH) functional group attached to at least one carbon atom. The alcohol may be a monohydric alcohol and may include at least one carbon atom, for example methanol. The alcohol may include at least two carbon atoms (e.g. ethanol). In other aspects, the alcohol comprises at least three carbons (e.g. isopropyl alcohol). The alcohol may include at least four carbon atoms (e.g., butanol), or at least seven carbon atoms (e.g., benzyl alcohol). The example payload may include no more than 50% (v/v) of the alcohol, more preferably, the payload comprises 2-45% (v/v) of the alcohol, 5-40% of the alcohol, and 10-40% of the alcohol. The aqueous solution may include 20-30% (v/v) of the alcohol.

In some aspects of the present subject matter, the virus is in an isotonic solution or buffer.

In some examples, “S Buffer” includes a hypotonic physiological buffered solution (78 mM sucrose, 30 mM KCl, 30 mM potassium acetate, 12 mM HEPES) for 5 min at 4° C. (Medepalli K. et al., Nanotechnology 2013; 24(20); incorporated herein by reference in its entirety). In some examples, potassium acetate is replaced with ammonium acetate in the S Buffer. S buffer is further described in international application WO 2016/065341, e.g., at ¶ [0228]-[0229] and incorporated herein by reference in its entirety.

According to the present subject matter, the aqueous solution may include at least one salt. The salt may be selected from NaCl, KCl, Na₂HPO₄, C₂H₃O₂NH₄ and KH₂PO₄. For example, KCl concentration ranges from 2 mM to 500 mM. In some preferred embodiments, the concentration is greater than 100 mM, e.g., 106 mM.

In examples, the aqueous solution comprises 32.5 mM, potassium chloride (KCl) 106 mM, Hepes 5 mM, ethanol, (EtOH) 12% v/v, and water for injection (WFI).

According to example methods of the present subject matter, the aqueous solution may include a sugar (e.g., a sucrose, or a disaccharide). According to example methods, the payload comprises less than 121 mM sugar, 6-91 mM, or 26-39 mM sugar. Still further, the aqueous solution (e.g., including the virus) includes 32 mM sugar (e.g., sucrose). Optionally, the sugar is sucrose and the payload comprises 6.4, 12.8, 19.2, 25.6, 32, 64, 76.8, or 89.6 mM sucrose.

According to example methods of the present subject matter, aqueous solution (e.g., including the virus) payload may include a buffering agent (e.g. a weak acid or a weak base). The buffering agent may include a zwitterion. According to example methods, the buffering agent is 4-(2-hydroxyethyl)-1-piperazineethanesulfonic acid. The aqueous solution (e.g., including the virus) may comprise less than 19 mM buffering agent (e.g., 1-15 mM, or 4-6 mM or 5 mM buffering agent). According to example methods, the buffering agent is 4-(2-hydroxyethyl)-1-piperazineethanesulfonic acid and the payload comprises 1, 2, 3, 4, 5, 10, 12, 14 mM 4-(2-hydroxyethyl)-1-piperazineethanesulfonic acid. Further preferably, the aqueous solution (e.g., including the virus) comprises 5 mM 4-(2-hydroxyethyl)-1-piperazineethanesulfonic acid.

According to example methods of the present subject matter, the aqueous solution (e.g., including the virus) includes ammonium acetate. The aqueous solution (e.g., including the virus) may include less than 46 mM ammonium acetate (e.g., between 2-35 mM, 10-15 mM, ore 12 mM ammonium acetate). The aqueous solution (e.g., including the virus) may include 2.4, 4.8, 7.2, 9.6, 12, 24, 28.8, or 33.6 mM ammonium acetate.

The volume of aqueous solution performed by gas propelling the aqueous solution may include compressed air (e.g. ambient air), other implementations may include inert gases, for example, helium, neon, and argon.

In certain aspects of the present subject matter, the population of cells may include adherent cells (e.g., lung, kidney, immune cells such as macrophages) or non-adherent cells (e.g., suspension cells).

In certain aspects of the present subject matter, the population of cells may be substantially confluent, and substantially may include greater than 75 percent confluent. In preferred implementations, the population of cells may form a single monolayer.

In aspects, contacting the population of cells with the volume of aqueous solution may be performed by gas propelling the aqueous solution to form a spray. In certain embodiments, the population of cells is in contact with said aqueous solution for 0.01-10 minutes (e.g., 0.1 10 minutes) prior to adding a second volume of buffer or culture medium to submerse or suspend said population of cells.

In various embodiments, the population of cells includes at least one of primary or immortalized cells. For example, the population of cells may include mesenchymal stem cells, lung cells, neuronal cells, fibroblasts, human umbilical vein (HUVEC) cells, and human embryonic kidney (HEK) cells, primary or immortalized hematopoietic stem cell (HSC), T cells, natural killer (NK) cells, cytokine-induced killer (CIK) cells, human cord blood CD34+ cells, B cells. Non limiting examples of T cells may include CD8+ or CD4+ T cells. In some aspects, the CD8+ subpopulation of the CD3+ T cells are used. CD8⁺ T cells may be purified from the PBMC population by positive isolation using anti-CD8 beads or by negative selection using anti-CD4 beads. In some aspects primary NK cells are isolated from PBMCs, cord derived NKs, iPSC-derived NKs, and GFP mRNA may be delivered by platform delivery technology. In additional aspects, NK cell lines, e.g., NK92 may be used.

Cell types also include cells that have previously been modified for example T cells, NK cells and MSC to enhance their therapeutic efficacy. For example: T cells or NK cells that express chimeric antigen receptors (CAR T cells, CAR NK cells, respectively); endosomes; cells that are transduced, and endosomes derived from them eg. MSCs. T cells that express modified T cell receptor (TCR); MSC that are modified virally or non-virally to overexpress therapeutic proteins that complement their innate properties (e.g. delivery of Epo using lentiviral vectors or BMP-2 using AAV-6) (reviewed in Park et al, Methods, 2015 August; 84-16.); MSC that are primed with non-peptidic drugs or magnetic nanoparticles for enhanced efficacy and externally regulated targeting respectively (Park et al., 2015); MSC that are functionalised with targeting moieties to augment their homing toward therapeutic sites using enzymatic modification (e.g. Fucosyltransferase), chemical conjugation (eg. modification of SLeX on MSC by using N-hydroxy-succinimide (NHS) chemistry) or non-covalent interactions (eg. engineering the cell surface with palmitated proteins which act as hydrophobic anchors for subsequent conjugation of antibodies) (Park et al., 2015). For example, T cells, e.g., primary T cells or T cell lines, that have been modified to express chimeric antigen receptors (CAR T cells) may further be treated according to the invention with gene editing proteins and or complexes containing guide nucleic acids specific for the CAR encoding sequences for the purpose of editing the gene(s) encoding the CAR, thereby reducing or stopping the expression of the CAR in the modified T cells. In other examples, the method and system herein is used for editing different genes in the modified cells to enhance the activity of the CAR-T cell, e.g., editing PD-1 to allow the CAR-T cells to evade an immune system checkpoint blockade. In other examples, the method and system herein is used to engineer mixtures of cell types in a single step. For example, mixtures of different T cell populations, or mixtures of different modified T cells, or mixtures of T cells and NK cells are used.

Aspects of the present invention relate to the expression viral delivery of gene editing compounds and complexes to cells and tissues, such as delivery of Cas-gRNA ribonucleoproteins for genome editing in primary human T cells, hematopoietic stem cells (HSC), and mesenchymal stromal cells (MSC). In some example, mRNA encoding such proteins are delivered to the cells.

In some embodiments, the gene editing composition comprises a gene editing protein, and the gene editing protein is a zinc finger nuclease (ZFN), a transcription activator-like effector nuclease (TALEN), a Cas protein, a Cre recombinase, a Hin recombinase, or a Flp recombinase. In additional embodiments, the gene editing protein may be a fusion proteins that combine homing endonucleases with the modular DNA binding domains of TALENs (megaTAL). For example, megaTAL may be delivered as a protein or alternatively, a mRNA encoding a megaTAL protein is delivered to the cells.

Various aspects of the CRISPR-Cas system are known in the art. Non-limiting aspects of this system are described, e.g., in U.S. Pat. No. 9,023,649, issued May 5, 2015; U.S. Pat. No. 9,074,199, issued Jul. 7, 2015; U.S. Pat. No. 8,697,359, issued Apr. 15, 2014; U.S. Pat. No. 8,932,814, issued Jan. 13, 2015; PCT International Patent Application Publication No. WO 2015/071474, published Aug. 27, 2015; Cho et al., (2013) Nature Biotechnology Vol 31 No 3 pp 230-232 (including supplementary information); and Jinek et al., (2012) Science Vol 337 No 6096 pp 816-821, the entire contents of each of which are incorporated herein by reference.

Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, or homologs thereof, or modified versions thereof. These enzymes are known; for example, the amino acid sequence of S. pyogenes Cas9 protein may be found in the SwissProt database under accession number Q99ZW2 and in the NCBI database as under accession number Q99ZW2.1. UniProt database accession numbers AOAOG4DEU5 and CDJ55032 provide another example of a Cas9 protein amino acid sequence. Another non-limiting example is a Streptococcus thermophilus Cas9 protein, the amino acid sequence of which may be found in the UniProt database under accession number Q03JI6.1. In some embodiments, the unmodified CRISPR enzyme has DNA cleavage activity, such as Cas9. In certain embodiments the CRISPR enzyme is Cas9, and may be Cas9 from S. pyogenes or S. pneumoniae. In various embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, a vector encodes a CRISPR enzyme that is mutated to with respect to a corresponding wild-type enzyme such that the mutated CRISPR enzyme lacks the ability to cleave one or both strands of a target polynucleotide containing a target sequence. For example, an aspartate-to-alanine substitution in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A. In aspects of the invention, nickases may be used for genome editing via homologous recombination.

In certain embodiments, a Cas9 nickase may be used in combination with guide sequence(s), e.g., two guide sequences, which target respectively sense and antisense strands of the DNA target. This combination allows both strands to be nicked and used to induce NHEJ.

As a further example, two or more catalytic domains of Cas9 (RuvC I, RuvC II, and RuvC III) may be mutated to produce a mutated Cas9 substantially lacking all DNA cleavage activity. A D10A mutation may be combined with one or more of H840A, N854A, or N863A mutations to produce a Cas9 enzyme substantially lacking all DNA cleavage activity. In certain embodiments, a CRISPR enzyme is considered to substantially lack all DNA cleavage activity when the DNA cleavage activity of the mutated enzyme is less than about 25%, 10%, 5%, 1%, 0.1%, 0.01%, or lower with respect to its non-mutated form. Other mutations may be useful; where the Cas9 or other CRISPR enzyme is from a species other than S. pyogenes, mutations in corresponding amino acids may be made to achieve similar effects.

In certain embodiments, a protein being delivered (such as a Cas protein or a variant thereof) may include a subcellular localization signal. For example, the Cas protein within a RNP may comprise a subcellular localization signal. Depending on context, a fusion protein comprising, e.g., Cas9 and a nuclear localization signal may be referred to as “Cas9” herein without specifying the inclusion of the nuclear localization signal. In some embodiments, the payload (such as an RNP) comprises a fusion-protein that comprises a localization signal.

For example, the fusion-protein may contain a nuclear localization signal, a nucleolar localization signal, or a mitochondrial targeting signal. Such signals are known in the art, and non-limiting examples are described in Kalderon et al., (1984) Cell 39 (3 Pt 2): 499-509; Makkerh et al., (1996) Curr Biol. 6 (8):1025-7; Dingwall et al., (1991) Trends in Biochemical Sciences 16 (12): 478-81; Scott et al., (2011) BMC Bioinformatics 12:317 (7 pages); Omura T (1998) J Biochem. 123(6):1010-6; Rapaport D (2003) EMBO Rep. 4(10):948-52; and Brocard & Hartig (2006) Biochimica et Biophysica Acta (BBA)—Molecular Cell Research 1763(12):1565-1573, the contents of each of which are hereby incorporated herein by reference. In various embodiments, the Cas protein may comprise more than one localization signals, such as 2, 3, 4, 5, or more nuclear localization signals. In some embodiments, the localization signal is at the N-terminal end of the Cas protein and in other embodiments the localization signal is at the C-terminal end of the Cas protein.

In some embodiments, an enzyme coding sequence encoding a CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis.

Accordingly, genes can be tailored for optimal gene expression in a given organism based on codon optimization. Codon usage tables are readily available, for example, at the “Codon Usage Database”, and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a CRISPR enzyme corresponding to the most frequently used codon for a particular amino acid.

In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. In some embodiments, the degree of complementarity is 100%. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In certain embodiments, a guide sequence is less than about 75, 50, 45, 40, 35, 30, 25, 20, 15, 12, or fewer nucleotides in length. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions.

CRISPR-Cas technology which facilitates genome engineering in a wide range of cell types is evolving rapidly. It has recently been shown that delivery of the Cas9-gRNA editing tools in the form of ribonucleoproteins (RNPs) yields several benefits compared with delivery of plasmids encoding for Cas9 and gRNAs. Benefits include faster and more efficient editing, fewer off-target effects, and less toxicity. RNPs have been delivered by lipofection and electroporation but limitations that remain with these delivery methods, particularly for certain clinically relevant cell types, include toxicity and low efficiency. Accordingly, there is a need to provide a delivery approach for delivering biologically relevant payloads, e.g., RNPs, across a plasma membrane and into cells. “Cargo” or “payload” are terms used to describe a microorganism, e.g., a non-bacterial microorganism such as a virus, a compound, or composition that is delivered via an aqueous solution across a cell plasma membrane and into the interior of a cell.

The current subject matter relates to delivery technology that facilitates delivery of a broad range of payloads to cells with low toxicity. Genome editing may be achieved by delivering RNPs to cells using some aspects of the current subject matter. Levels decline thereafter until Cas9 is no longer detectable. The delivery technology per se does not deleteriously affect the viability or functionality of Jurkat and primary T cells. The current subject matter enables gene editing via Cas9 RNPs in clinically relevant cell types with minimal toxicity.

The transient and direct delivery of CRISPR/Cas components such as Cas and/or a gRNA has advantages compared to expression vector-mediated delivery. For example, an amount of Cas, gRNA, or RNP can be added with more precise timing and for a limited amount of time compared to the use of an expression vector. Components expressed from a vector may be produced in various quantities and for variable amounts of time, making it difficult to achieve consistent gene editing without off-target edits. Additionally, pre-formed complexes of Cas and gRNAs (RNPs) cannot be delivered with expression vectors.

In one aspect, the present subject matter describes cells attached to a solid support, (e.g., a strip, a polymer, a bead, or a nanoparticle). The support or scaffold may be a porous or non-porous solid support. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present subject matter. The support material may have virtually any possible structural configuration. Thus, the support configuration may be spherical, as in a bead, or cylindrical, as in the inside surface of a test tube, or the external surface of a rod. Alternatively, the surface may be flat such as a sheet, or test strip, etc. Preferred supports include polystyrene beads.

In other aspects, the solid support comprises a polymer, to which cells are chemically bound, immobilized, dispersed, or associated. A polymer support may be a network of polymers, and may be prepared in bead form (e.g., by suspension polymerization). The cells on such a scaffold can be sprayed with payload containing aqueous solution according to the invention to deliver desired compounds to the cytoplasm of the scaffold. Exemplary scaffolds include stents and other implantable medical devices or structures.

### EXAMPLES

The following examples illustrate certain specific embodiments of the invention and are not meant to limit the scope of the invention.

Embodiments herein are further illustrated by the following examples and detailed protocols. However, the examples are merely intended to illustrate embodiments and are not to be construed to limit the scope herein. The contents of all references and published patents and patent applications cited throughout this application are hereby incorporated by reference.

### Example 1: Delivery of Virus as Payload

Non-bacterial microorganisms, e.g., viruses were delivered to eukaryotic cells. In some examples, a commercially available virus, e.g., an Adeno-associated virus (AAV), a lentivirus or a retrovirus is employed. For example, lentivirus virus contains a nucleic acid encoding a model (test) cargo such as GFP. The virus (encoding for GFP) is delivered using the method and system herein system described herein.

A multiplicity of infection (MOI) (alternatively, the number of virions (virus particles) added per cell in an infection) of 0.1, 1 10 and 100 was performed. The volume of method and system herein to number of cells was evaluated.

A range of MOIs was tested, and a range of volumes from about 1 μL to about 1000 mL was tested.

Additionally, different membrane holder sizes were tested to allow for removal of media from a range of cell numbers 1×10⁵-1×10⁷ cells. Cells are cultured following transduction and will grow to the capacity needed for cell therapy, for example up to 10⁹ cells.

A commercially available lentiviral vector encoding GFP, transduced using for example, Spinoculation, was used as a control. The control is used in accordance with a standard protocol for spinoculation of lentiviral vectors to suspension cells. In examples, a commercially available Spinoculation method was used for transduction of suspension cells (Jurkat T cells, PBMC, PBL, B cells etc.).

Transduction of Suspension Cells and Evaluation/Analysis

For normal transduction of suspension cells, the virus was added to activated T cells in culture flasks and bags as a control. The cells are cultured after introduction of the virus, and the virus is washed. After a number of days, the cells are harvested and run on a flow cytometer to assess viability and % transduction (GFP-encoding virus).

The longevity of transduction efficiency was monitored over 14 days.

The vector copy number (VCN) was evaluated to determine a low copy/cell number, for example, 0.5-1 copy/cell. Exemplary release criteria include (0.5-5 for Levine 2006). The VCN is established using quantitative PCR.

The phenotypic changes were evaluated by flow cytometry. For example, changes including maintenance of naïve, central memory, effector memory were evaluated.

Transduction to T Cells, and Other Cell Types is Performed

Viral delivery to T cells in multiple activation states was also evaluated. For example, it is commonplace to transduce activated T cells. Studies were carried out to determine whether naïve T cells can be used, thus reducing COGs (Cost of Goods). There is no need for activation and thus an advantage of the methods described herein include the reduction of processing time. In other examples, delivery to previously modified T cells, e.g., CAR-T cells or gene edited T cells is also evaluated.

Transduction

Additionally, a standard transduction of the virus was performed. For example, HEK 293 adherent cells were used. This method removes the necessity to remove media through a filter. For example, cells can be seeded on a 6 well-plate and the media is removed manually to expose cells, and then the cells are permeabilized directly on the plate. Additionally, some of the issues regarding getting the system into a viral containment lab and isolation of all equipment are eliminated.

AAV, retrovirus, and the like are also transduced using such methods. Lentivirus is advantageous because it is the most simple ex-vivo transduction method, and AAV represents a common virus used for commercial aspects.

### Example 2: Delivery of Virus Via Spray

The virus can be delivered via the SOLUPORE™ apparatus. For example, a small, medium or large-system, including the described engineering iterations including various membranes, device size, etc.

In this method, the media is removed, temporarily, from cells forming a monolayer of cells to allow dropletised virus to come in close contact with cells for a specified incubation, after which media is returned to cells and cells are cultured for downstream applications.

The method and system herein may include a filter membrane (with or without a drain disk) through which media is temporarily removed from cells (by centrifugation, gravity flow or vacuum). Following monolayer formation, various cargo is aerosolised in a buffer and the droplets applied onto the cells in a controlled manner (volumes, heights etc. variable). Various cargo include mRNA, DNA, CRISPR RNPs and viral particles (lenti viral, retro viral, AAV etc.) to enable gene transfer to cells or editing of genetic material in cells.

In some implementations the process involve T cells, NK cells and the like.

In some implementations the cells are addressed using the SMA nebuliser, Conikal nebulizer, and the like.

The system is used for transducing T cells. Additionally, the system is used for the delivery editing cargoes and virus simultaneously or serially. This system is also used for T cell engineering using RNA, DNA and siRNA. This system is further used to knock in or knock out genes, which can be done in isolation or in parallel with introducing genetic information using viruses. This system is used to edit cells using a multitude of cell editing platforms—CRISPR/Cas9, Cas12, MegaTALs, TALENs, ZFNs etc—all of which could be delivered alone, done in parallel with virus or sequentially.

In some implementations the cells are addressed by multiple nebulisers at once or in sequence. In other iterations, one nebuliser is used in isolation. Using the nebuliser to aerosolise viral particles improves contact between cells and virus, and improves transduction. Virus is dropletised and lands on surface of a membrane where cells are located. Dropletizied virus allows for much lower volumes being administered to cells. Following a short incubation media would be replaced. This media may also contain a low dose of virus.

Multiple cell types are tested. Additionally, various medias and additives, e.g., cytokines are added, including for example polybrene or PGE2 to enhance transduction. Other commercially available fibronectin peptides (Retronectin) or Lentiboost are also added to enhance the interaction between virus and cells, and are added in different iterations.

The rates of spray/duration/pressure are also evaluated. Recovery of cells by different methods (e.g., bottom up/pipette) are tested.

Cargoes for genetic engineering may be suspended in a variety of buffers, however none of this limits the scope of this patent, which contains many variations of what cargoes can be delivered to what cell types.

Furthermore, transduction of T cells can be very variable, and with this controlled method of delivery this could make viral transduction more consistent, and thus more advantageous.

### Example 3: Gene Therapy Viral Vectors

The tables below depict the main differences between the various types of virus used in gene therapy of eukaryotic cells.

Overview of common viruses used for generating gene therapy viral vectors

| Parameter | Retrovirus | Lentivirus | AAV | Adenovirus |
|---|---|---|---|---|
| Coat | Enveloped | Enveloped | Non-enveloped | Non-enveloped |
| Packaging | 8 | 8 | ~4.5 | 7.5 |
| capacity (Kb) | | | | |
| Tropism/ | Dividing | Broad | Broad excluding | Broad |
| infection | cells | | hematopoietic | |
| | | | stem cells | |
| Inflammatory | Reduced | Reduced | Reduced | High |
| potential | | | | |
| Host genome | Integrating | Integrating | Integrating/ | Non-integrating |
| interaction | | | non-integrating | |
| Transgene | Long lasting | Long | Potentially | Transient or |
| expression | | lasting | long-lasting | long-lasting |
| | | | | depending on |
| | | | | immunogenicity |

Overview of Aderhent and Suspension cells in viral vector use.

| Cells | Cell Culture System | Viral Vector | Titer* |
|---|---|---|---|
| Adherent HEK- | 10 cm dish/75 cm² | Lentivirus | 1-2 × 10⁸ TU/ml |
| 293, HEK-293T | HYPERflask ®/HYPERstack ® | Lentivirus | 1-2 × 10⁸ TU/ml |
| | CS10 ®/CF10 ® | AAV | 10¹¹-10¹³ VG/ml |
| | Fixed-bed bioreactor | AAV | 10¹⁴-10¹⁶ total VG |
| | (iCELLS ®) | | |
| Suspension | Shaker Flask | AAV | 10⁹-10¹⁰ VP/ml |
| HEK-293, HEK- | Shaker Flask | Lentivirus | 2 × 10⁷-10¹⁰ VP/ml |
| 293T | Bioreactor | AAV | 10⁹-10¹⁰ VG/ml |
| | Bioreactor | Lentivirus | 10⁷ TU/ml |

The retrovirus has many disadvantages and thus AAV and lentiviruses are more widely used.

### Example 4: Viral Delivery to a Population of Cells Using the SOLUPORE™ Process

The SOLUPORE™ process enables the delivery of a wide range of cargo to adherent and suspension cells in vitro and ex vivo. To date, these cargos have consisted of molecules such as nucleic acids and proteins and particles such as Qdots. The data herein show that the SOLUPORE™ process can be used to deliver a non-bacterial microorganism such as a virus, (e.g., a lentivirus) to T cells with efficiency higher than standard control transduction.

### Viruses as Vectors

Viruses are used as vectors for delivery of nucleic acids to cells, because they can naturally infect human cells. Prior to entry, a virus must attach to a host cell. Attachment is achieved when specific proteins on the viral capsid or viral envelope bind to specific receptor proteins on the cell membrane of the target cell. Depending on the type of virus, entry into the cell can occur in different ways. Viruses with a viral envelope can enter the cell by membrane fusion where the cell membrane is punctured and made to further connect with the unfolding viral envelope. Viruses with no viral envelope can enter by endocytosis (FIG. 2). Other viruses such as bacteriophages attach to the cell surface, and only the viral genome is injected into the host cells.

### Immune Cell Engineering

Different types of viruses have different features which need to be considered if they are being used to transduce cells ex vivo for clinical applications. The main features are: immunogenicity; target cell type; payload capacity; ability to transduce non-dividing versus dividing cells; transient versus stable genome integration (Table 5).

Summary of viruses used for gene delivery applications.

| Virus | Description | Advantages | Disadvantages |
|---|---|---|---|
| Adenoviruses (AdVs) | non-enveloped dsDNA- | efficient in a broad range of | high immunogenicity; |
| | virus able to carry ≤8 kbp | host cells | transient expression |
| | DNA | | |
| Adeno-associated viruses | non-enveloped recombinant | efficient in a broad range of | small carrying capacity |
| (AAVs) | ssDNA-virus with a small | host cells; non- | |
| | carrying capacity (≤4 kbp) | inflammatory/pathogenic | |
| Retroviruses | enveloped ssRNA-carrying | long-term expression | limited tropism to dividing |
| | virus with ≤8 kbp RNA | | cells; random integration |
| | capacity | | |
| Lentiviruses | enveloped ssRNA-carrying | efficient in a broad range of | potential oncogenic |
| | virus with ≤8 kbp RNA | host cells; long-term | responses |
| | capacity | expression | |
| Herpes simplex viruses | enveloped dsDNA-virus | efficient in a broad range of | potential inflammatory |
| (HSV)-1 large packing | with >30 kbp carrying | host cells | responses; transient |
| capacity; | capacity | | expression |

Pharmaceutics 2020, 12, 183, incorporated herein by reference in its entirety

For immune cell therapy applications such as delivery of chimeric antigen receptor (CAR) constructs for generation of CAR-T and CAR-NK cells, gammaretroviruses and lentiviruses are typically used because they are capable of transducing immune cells and because they result in stable integration into the genome. For example, the first two approved CAR-T cell products, Kymriah and Yescarta, were engineered using lentivirus and gammaretrovirus vectors respectively (Poorebrahim M et al. Crit Rev Clin Lab Sci. 2019 September; 56(6):393-419). As with most viral vectors, these vectors were modified in ways that rendered the viruses replication-incompetent and improved cell targeting efficiencies.

For other immune cell engineering applications such as gene editing, AAV vectors are widely used. While wild type AAVs can stably integrate into chromosome 19, AAV-based gene therapy vectors have been modified to prevent integration and instead form episomal concatemers in the host cell nucleus. In non-dividing cells, these concatemers remain intact for the life of the host cell. In dividing cells, AAV DNA is lost through cell division, since the episomal DNA is not replicated along with the host cell DNA. So AAV vectors are often used to deliver editing systems such as the CRISPR/Cas9 system or donor template DNA for gene editing. In these cases, while the gene edit is permanent, it can be desirable to only transiently express the gene editing tools in order to limit non-specific off-target gene editing that can occur if the tools are present in the cells for extended time periods.

### Lentiviruses

Gammaretroviruses and lentiviruses are subtypes of retroviruses, which contain an RNA genome that is converted to DNA in the transduced cell by a virally encoded enzyme called reverse transcriptase. For retroviruses, entry into the cell is followed by a process of uncoating whereby several viral proteins dissociate from the viral core. The viral RNA is reverse transcribed to double stranded DNA. Viral proteins then complex with the proviral DNA to bring about nuclear import and integration into the host genome. The process of integration is assisted by crucial viral proteins, such as integrase, and endogenous host cell transcription factors.

Lentiviral vectors derived from the human immunodeficiency virus (HIV-1) have become major tools for gene delivery into mammalian cells and replication-deficient recombinant lentiviruses are widely used in research and clinical applications. While the modified lentivirus is still able to infect cells, the essential genes for producing new viral particles are no longer present. Lentiviral vectors are regarded as attractive gene-delivery vehicles for several reasons: they offer long term gene expression via stable vector integration into host genome; they are capable of infecting both dividing and non-dividing cells; they are capable of infecting a broad range of cells including important target cell types for gene and cell therapies; they lack immunogenic viral proteins after vector transduction; they can deliver complex genetic elements such as intron-containing sequences; they are a relatively easy system for vector manipulation and production. Lentiviral vectors have a safer integration site profile than gammaretroviral vectors and are commonly used in clinical trials of CAR T cell therapies (McGarrity G. J. et al. J. Gene Med 2013; 15:78-82). Third-generation lentiviral vectors incorporate key safety features, further enhancing safety (Kim V. N. et al. J. Virol. 1998; 72:811-816 and Dull T. et al. J. Virol. 1998; 72:8463-8471).

The range of cell types that are transducible with retroviruses has been broadened by pseudotyping retroviruses with the envelope glycoprotein G of the vesicular stomatitis virus (VSV-G). VSV-G binds the ubiquitous membrane component phosphatidylserine, which enables the VSV-G pseudotyped virus to attach and transduce a much wider range of cells. Currently, most lentiviral vectors are pseudotyped with VSV-G to enable robust transduction into many cell types including neurons, lymphocytes, and macrophages.

### Quiescent Cells

Following their isolation from either healthy donors or patients, cells are activated and subsequently transduced by lentiviral vectors (LVs), the majority of which are pseudotyped with the glycoprotein G of vesicular stomatitis virus (VSV). The modified lymphocytes are then expanded and either used in functional in vitro assays or used for in vivo applications. Stable and efficient transduction of B cells by LVs is very difficult to achieve. Quiescent B cells are restrictive to transduction by conventional VSV G-pseudotyped LVs (VSV-LVs) due to a lack of low-density lipoprotein receptor (LDLR) expression and they must be activated prior to transduction. Efficient activation and culture of primary human B lymphocytes is complex, as it involves carefully titrated activating stimuli in combination with cytokines followed by co-cultivation with feeder cells. Even under optimal activation and culture conditions, transduction efficiencies with VSV-LVs are notoriously low. The combination of all these difficulties may serve as an explanation for the much lower number of clinical trials involving engineered B cells as compared to T cells.

In comparison, T lymphocyte manipulation by lentiviral transduction is easier to achieve. Still, the cells have to be activated prior to transduction with conventional VSV-LV, because, like B cells, they are otherwise not susceptible for transduction, again due to a lack of LDLR expression (X Geng, et al. Gene Therapy v. 21, pages 444-449(2014)). Mainly due to the paramount success of CAR T cell therapy, the protocols for T cell isolation, activation, lentiviral transduction, and expansion have been extensively improved in recent years. Current state-of-the-art T cell activation relies on stimulation of the TCR activation pathway via CD3- and CD28-specific antibodies in combination with cytokines such as IL-7 and IL-15.

The need for activation of lymphocytes prior to transduction with conventional LVs has disadvantages. It adds to the complexity of the overall procedure increasing duration and costs of the manufacturing process. In addition, the stimuli applied for activation in combination with the prolonged ex vivo culture likely changes the cells, which can negatively impact on the quality of the final product. As a result, naive cells could differentiate into less preferential phenotypes that exhibit a higher degree of exhaustion, lower proliferative capacity, shorter in vivo persistence, and less functionality. This can have very important implications for therapeutic success. For instance, it has been shown that a central memory (CD45RO+/CD45RA+/CD62L+) or stem cell memory (CD45RO+/CD45RA/CD62L+) phenotype is beneficial for T cell persistence and function in vivo. In this regard, a positive correlation of a CAR T cell central memory phenotype and a positive clinical response has been observed in several clinical studies, and, consequently, the infusion of purified central memory CAR T cells is now being considered. Likewise, a central memory phenotype leads to functionally superior TCR-modified T cells. Minimal manipulation of lymphocytes during genetic modification is thus of significant clinical relevance.

### Spinoculation

Centrifugal inoculation, or spinoculation, is widely used in virology research to enhance viral infection. The procedure involves centrifuging a mixture of virus and target cells at high speed for a prolonged period for example 800×g for 30 minutes at 32° C. It was thought that the method enhances transduction rates by concentrating virus at the cell membrane. However, it has been shown that spinoculation triggers dynamic actin and cofilin activity, probably resulting from cellular responses to centrifugal stress (Jia Guo, et al. J. Virology October 2011, p. 9824-9833). This actin activity also leads to the upregulation of cell membrane receptors that may enhance viral binding and entry. It has been suggested that spin-mediated enhancement cannot be explained simply by a virus-concentrating effect; rather, it is coupled with spin-induced cytoskeletal dynamics that promote receptor mobilization, viral entry, and postentry processes. Therefore, spinoculation may affect the biology of the target cell in unknown ways or in ways that are undesirable.

### Limitations of Viral Vectors

While viruses have been useful for genetic engineering of cells as described above, there are limitations to their utility.

High-cost manufacturing processes of Chimeric Antigen Receptor (CAR) T-cells therapies are prohibitively expensive. Due to the cost of virus, transduction is a major cost driver in CAR T-cell manufacturing. Several bioprocessing parameters have been identified as potentially playing a role in transduction efficiency, such as the physical proximity of lentivirus particles to T cells. This proximity could be manipulated through the number of cells and virus particles in the suspension; the periods of agitation to encourage homogeneity; and the surface-to-volume ratio in the transduction vessel. However, limited research has been performed on identifying and optimizing critical process parameters of transduction. During the SOLUPORE™ process, a small volume of delivery solution is applied directly onto exposed target cells. In this way, the cargo is brought directly in contact with the cells in a gentle manner. Delivering viruses to cells in this way leads to a concentration of material at the cell membrane. This process enhances viral attachment to the cell membrane and enhances the rate of entry into the cell making the process more efficient. In turn, small doses of virus are used and costs are reduced.

Because the SOLUPORE™ process is a gentle method of concentrating virus at the cell membrane, it has significant advantages to existing concentration methods such as spinoculation which can affect cell structure. Furthermore, unlike spinoculation, the SOLUPORE™ process is designed to be compatible with cell therapy manufacturing processes.

The concentration of viruses at the cell membrane also compensates for the low levels of expression of viral receptors on certain cell types such as unactivated T cells and thus enhances transduction efficiencies in these cells.

Efficiency of lentiviral vector transduction of unactivated T cells and B cells is typically very low. It is highly desirable to improve these efficiencies, and the SOLUPORE™ process provides a solution to tis problem with high efficiency rates coupled with conditions that are compatible with preservation of cell viability and function.

Viruses are only capable of delivering nucleic acid which means they are restricted in the type of cargo that they can deliver. If viruses could be co-delivered with other types of cargo, it could enhance the utility of viruses in the engineering on next-generation cell therapy products. However, there is currently no method that has been demonstrated to co-deliver viruses with other types of cargo. Again, the SOLUPORE™ process described herein provides a solution to this problem by permitting efficient delivery of numerous different cargo types sequentially or simultaneously. The following materials and methods were used to generate date described herein.

### LV-GFP Vector

The LV-GFP vector used here carries the vesicular stomatitis virus-G (VSV-G) envelope protein, known to target a wide variety of cell types.

### Stability of LV-GFP in Delivery Solution

The stability of LV-GFP in delivery solution was evaluated over an hour by assessing precipitation under a microscope.

### LV-GFP Delivery

Primary human PBMCs were thawed and activated for 3 days with Miltenyi CD3 and CD28 antibodies. After 3 days of activation culture the cells underwent the SOLUPORE™ process or static transduction with LV-GFP (MOI=2.5). Cells were recovered for 72 hours before GFP expression was assessed by flow cytometry.

### Stability of LV-GFP in Delivery Solution

Prior to the invention, the SOLUPORE™ process delivery had not been previously combined with a virus preparation so it was unknown whether there would be solubility problems.

No aggregation or precipitation was observed when the solution was viewed under the microscope during the stability analysis. Furthermore, certain other cargos have exhibited low levels of aggregation when combined with the delivery solution and have caused the Solupore® atomizer to block during spraying. When the delivery solution containing the virus was loaded into the Solupore® atomizer and sprayed, no blocking occurred at any point over the course of the study, further indicating that no aggregation or precipitation occurred.

### Cell Viability, Expansion and GFP Expression Following LV-GFP Delivery

LV-GFP was delivered to T cell cultures by the SOLUPORE™ process and compared with a standard static method of LV transduction.

The viability of cells was measured at various timepoints before and after the delivery of virus. At all timepoints, the viability of soluporated cells was comparable to that of control transduced cells (FIG. 3).

The cumulative fold expansion of the T cells was determined up to 96 hr after delivery of virus. The expansion of soluporated cells was comparable to that of control transduced cells (FIG. 4).

The expression of GFP was measured at day 3 and day 4 post-delivery. GFP expression efficiency was higher in soluporated T cells compared with control transduced cells (FIG. 5). At day 3, efficiency was 39.73±2.83% compared with 25.2±1.48% for soluporated cells and control transduced cells respectively. At day 4, efficiency was 40.27±2.67% compared with 26.83±1.38% for soluporated cells and control transduced cells respectively.

### Efficient Viral Delivery of Virus to a Population of Cells

The data herein demonstrated that the SOLUPORE™ process is compatible with delivery of virus to activated T cells. When mixed with the SOLUPORE™ process delivery solution, no precipitation or aggregation was observed. It was possible to spray the viral solution and the target cells were successfully transduced. The viability and the expansion rate of the soluporated T cells were unaffected.

GFP expression was higher in soluporated T cells compared with control transduced cells indicating that the SOLUPORE™ process enhances viral transduction of T cells.

Together these finding demonstrate that the SOLUPORE™ process is suitable for delivery of viruses to cells and is superior to standard methods.

Because the SOLUPORE™ process is suitable for delivery of virus, it is possible to use soluporaton in cell therapy manufacturing processes that involve viral transduction. Due to the cost of virus, transduction is a major cost driver in CAR T-cell manufacturing. Because the SOLUPORE™ process enhances viral transduction, it is now possible to use less virus to achieve similar levels of transduction efficiency and thus reducing costs.

Different cargos can be delivered simultaneously by the SOLUPORE™ process. The demonstration herein that the SOLUPORE™ process is compatible with viral delivery means that the SOLUPORE™ process can be used to co-deliver virus with other cargos. These other cargos could be other viruses or could be proteins, nucleic acids, small molecules or complexes thereof. The ability to co-deliver cargos means that engineering steps that would otherwise happen in different process steps can be combined into a single process step. This process has major benefits for manufacturing processes including cost, time and labor. In addition, fewer process steps means less handling and risk of contamination as well as simplifying the process. Alternatively, virus is delivered in sequence, before or after other cargos.

The SOLUPORE™ process enables delivery of cargo to unactivated T cells. Lentiviral vectors have very low transduction efficiency in unactivated T cells. Therefore, the SOLUPORE™ process increases the transduction efficiency of lentivirus in unactivated T cells.

A core feature of the SOLUPORE™ process device is its ability to facilitate changes of medium. When a solution containing cells is transferred into the device, the liquid can be drained away and replaced with different liquids. In this way, liquid handling steps are possible. Such liquid handling steps could include for example wash steps. With viral transduction and other cell manufacturing process, wash steps are often required. The Solupore® device enables integration of such steps into a manufacturing process.

The Solupore® technology is also scalable which means that viral transduction using this method could be carried out at small scale for early and pre-clinical work as well as larger scales for process development and clinical applications.

### Example 5: Delivery of Lentiviral Vectors (LV) to T-Cells by the SOLUPORE™—Process Using Solupore™. A Dataset Around LV Delivery by the SOLUPORE™ Process in Comparison to a Static Transduction Control was Generated

Viral delivery technology for cell and gene therapies were developed. The platform relies on reversible cell permeabilization for payload delivery using a functionally closed device. Using this approach, effective nucleic acid and gene editing was demonstrated in therapeutically relevant cell types with minimal impacts on cell viability, proliferation, gene expression or phenotype. Utility for delivering viral vector to target cells was also demonstrated. The data describes lentiviral vector delivery by the SOLUPORE™ process to T-cells.

### PBMC Isolation

Half of a fresh leukapheresis pack (Donor ID: RG1083) was obtained from StemCell Technologies. PBMCs were isolated and cryopreserved as described in “Isolation, Initiation and cell culture of PBMC derived T cells” (provided at Example 5). To generate a Peripheral Blood Mononuclear Cells (PBMC) cell bank, cells were counted, and viability was assessed by Nucleocounter NC-200. 61.5×10⁶ viable cells/mL were frozen in 1 mL aliquots at a controlled rate of −1° C./min to −100° C. using VIA Freeze. A total of 43 vials were banked and all frozen vials were transferred from VIA Freeze to liquid N₂ tank for permanent storage. Qualification of PBMC bank was performed by thawing 3 random vials and assessing cell viability and cell recovery upon thaw. Thawing procedure was carried out as described in “Isolation, Initiation and cell culture of PBMC derived T cells” (provided). For each vial, 4×10⁶ cells at a viable cell density of 1×10⁶ cells/mL were also seeded in a 6-well plate and activated using CD3 and CD28 antibodies. Cells were harvested and expression of CD3 and CD25 were assessed 3 days post-activation as described in “Cell Thaw, Culture and Preparation of cells for Experimental Use” (provided at Example 7).

### PBMC Thaw and Initiation

Complete media was prepared, and cryopreserved PBMCs were thawed and activated for 3 days in accordance with using protocol described in “Isolation, Initiation and cell culture of PBMC derived T cells” (provided at Example 5). Using an inverted microscope, a representative 10× image was taken to capture clumping and overall morphology of cells. To determine if CD3 and CD25 expression post-activation met release criteria for the SOLUPORE™ process, activated PBMC-initiated T-cells were harvested as outlined in Example 8 and stained with CD3- and CD25-conjugated antibodies for flow acquisition and analysis. Expression of CD3 and CD25 were verified to be >90% before the cells were released for experimental use.

### Lentivirus (LV-eGFP; “Enhanced GFP”))

Three lots of LV-eGFP supplied in 50 μL aliquots were obtained from Tailored Genes and stored at −80° C. LV batches were tittered and adapted for K562 cells.

To investigate the stability of LV in the Delivery Solution formulation, 75 μL of Delivery Solution with LV-eGFP as payload was prepared in a 96-well plate. 4×, 10× and 20× images were taken using an inverted microscope every hour for 4 hours to assess possible precipitation. The delivery solution, includes, sucrose 32.5 mM, KCl 106 mM, Hepes 5 mM, EtOH 12% v/v, and Water for injection.

Required number of aliquots were thawed on ice and briefly centrifuged to collect loose liquid on the side of the vials. LV-eGFP aliquots were pooled before use to ensure there was sufficient volume for the experiment. Once Payload Delivery Solution was prepared in 0, remaining LV-eGFP was kept at RT until static transductions were performed.

### Specifications of the Three LV-eGFP Lots Used in all Experimental Runs.

| | | Tailored Genes | | CCRM | |
|---|---|---|---|---|---|
| | | | Titering | | Titering |
| Lot # | Date Prepared | Titer (TU/mL) | Method | Titer (TU/mL) | Method |
| GFP181025H7 | 2018-10-25 | 4.40 × 10⁹ | Flow | 1.27 × 10⁹ | ddPCR |
| | | | Cytometry | | |
| GFP181025H8 | 2018-10-25 | 3.27 × 10⁹ | Flow | 0.93 × 10⁹ | ddPCR |
| | | | Cytometry | | |
| GFP20TG007 | 2020-03-25 | 1.31 × 10⁹ | Q-PCR | — | — |

### Preparation of PBMC-Initiated T-Cells

As outlined in “Cell Thaw, Culture and Preparation of cells” (provided at Example 7), the required number of PBMC-initiated T-cells were pelleted by centrifugation and resuspended in basal media (CTS OpTmizer T Cell Expansion SFM containing supplement) to yield the desired cell density listed for each experiment. Post-dilution cell counts were performed, and 30 mL aliquots of cell suspension were prepared in 50 mL Falcon tubes and kept in a 37° C. incubator until use. Immediately before the SOLUPORE™ process, each sample was weighed and assessed for both cell count and viability to ensure cells remained at the desired cell density. Each sample was then transferred to a 50 mL syringe in preparation for the SOLUPORE™ process as described in “the SOLUPORE™ process of PBMC Initiated T Cell Cultures” (described herein).

### Static Transduction

For each Multiplicity of Infection (MOI) condition, the required number of T-cells was diluted with complete media to yield the desired volume and post-dilution cell counts were performed to confirm cell density. The prepared cell suspension was then aliquoted into Nunc™ EasYFlasks™ TC-treated T25 flask—3 technical replicates and 1 untreated control per MOI condition. The cells were kept in a 37° C., 5% CO₂ incubator until static transductions were performed.

Static Transduction parameters tested in each experiment.

| Parameter | Run 1/2 | Run 3/4 |
|---|---|---|
| Viable Cell Number/Aliquot | 10 × 10⁶ (MOI 2.5) | 1.8 × 10⁶ (MOI 2.5) |
| | | 0.9 × 10⁶ (MOI 5) |
| Volume | 20 mL | 3 mL |
| Target Viable Cell Density | 0.5 × 10⁶ cells/mL | 0.6 × 10⁶ cells/mL (MOI 2.5) |
| | (MOI 2.5) | 0.3 × 10⁶ cells/mL (MOI 5) |
| Culture Vessel | T25 TC flask | T25 TC flask |
| Vessel Orientation | Upright | Flat |
| Volume of LV-eGFP | 25 μL | 4.5 μL |
| Untreated Control/MOI Condition | 1 | 1 |
| Technical Replicates/MOI Condition | 3 | 3 |

To capture any potential degradation of the LV over time, LV was added to each static transduction sample in parallel with the completion of a the SOLUPORE™ process. Immediately after virus addition, the flasks were returned to a 37° C., 5% CO₂ incubator.

For each run, complete media was doubled one day after viral delivery. Given the larger volumes for Run 1/2, the samples were transferred to a T75 flask and cultured lying flat. For Run 3/4, 3 mL of complete media was added to each T25 flask. For Run 1, final analyses were performed three days after the SOLUPORE™ process. For Run 2 onwards, three-day post-infection cultures were diluted to a VCD of 0.5×10⁶ cells/mL after timepoint analyses were performed to allow for further analysis on the next day. Post-dilution cell counts were carried out to track cumulative fold expansion up to four days after the SOLUPORE™ process. All post-recovery and post-the SOLUPORE™ process timepoint analyses were completed.

### Assembly, Calibration, and the SOLUPORE™ Process Using Solupore™ System

Following “the SOLUPORE™ process of PBMC Initiated T Cell Cultures” (described herein), sterilized components were assembled aseptically in a Biosafety Cabinet (BSC). A pressure leak test was completed to ensure the device was properly sealed and a pressure profile was saved. Calibration and stop solutions were prepared according to “the SOLUPORE™ process of PBMC Initiated T Cell Cultures” (described at Example 6) and placed on ice until use.

Due to an increase in payload volume to achieve a MOI of 2.5, the system was calibrated to deliver 75-80 μL in Run 1/2. As the LV-eGFP payload solution had a higher viscosity than the calibration solution, there was a substantial amount of Payload Delivery Solution remaining after both runs. In order to deliver the desired amount of LV, the system was subsequently calibrated to deliver 95-100 μL to adjust for the insufficient spray volume. Parameters tested for the SOLUPORE™ process in each experiment.

The SOLUPORE ™ process parameters tested in all experiments.

| Task | Parameter | Run 1/2 | Run 3/4 |
|---|---|---|---|
| Calibration | Target Delivery Volume | 75-80 μL | 95-100 μL |
| | Measurement by scale | 75-80 mg | 95-100 mg |
| the | Number of cells for the | 24 × 10⁶ | 24 × 10⁶ (MOI 2.5) |
| SOLU- | SOLUPORE ™ process | (MOI 2.5) | 12 × 10⁶ (MOI 5) |
| PORE ™ | Harvest Volume | 20 mL | 20 mL |
| process | Culture Volume | 19-25 mL | 3 mL |
| | Culture Vessel | T25 TC flask | T25 TC flask |
| | Vessel Orientation | Upright | Flat |
| | Technical Replicates/ | 3 | 3 |
| | MOI Condition | | |

Assuming a viral titer of 1×10 TU/mL, the volume of LV-eGFP payload (μL) required for each spray was calculated as follows for all experiments:

$${V}_{Payload} = No . of Cells for Soluporation \times \frac{MOI}{1 \times 1 {0}^{9} TU / mL} \times \frac{1000 μL}{mL}$$

Based on the total delivery volume, the final concentration of LV-eGFP payload (%) was determined by:

$$\% L V = \frac{{V}_{payload}}{{V}_{delivery}} \times 100 \%$$

The total volume of Delivery Solution required for each experiment was derived using the following calculation:

$$[ {V}_{Spray} ( 75 μL ) + {V}_{20 \% Excess} ( 15 μL ) ] \times No . of Sprays + {V}_{Atomiser Priming} ( 75 μL ) + {V}_{D e a d} ( 100 μL )$$

For all experiments, Delivery Solution was formulated based on the final concentration of each component listed below:

Payload and Delivery Solution composition for Syringe &
Elveflow Reservoirs. % WFI (water for injection)
was adjusted to reflect an increase in LV-eGFP composition.

| Components of | |
|---|---|
| Delivery Solution | Final Concentration |
| WFI | 3% |
| Ethanol | 12% |
| 20X S Buffer | 5% |
| LV-eGFP | 80% |

For all the SOLUPORE™ process, cells were loaded, soluporated, and harvested in 20 mL of complete media. Inter-sample cleaning and priming were performed as described in “the SOLUPORE™ process of PBMC Initiated T Cell Cultures” (described herein). Post-recovery and post-the SOLUPORE™ process timepoint analyses were completed.

In some examples, “S Buffer” includes a hypotonic physiological buffered solution (78 mM sucrose, 30 mM KCl, 30 mM potassium acetate, 12 mM HEPES) for 5 min at 4° C. (Medepalli K. et al., Nanotechnology 2013; 24(20); incorporated herein by reference in its entirety). In some examples, potassium acetate is replaced with ammonium acetate in the S Buffer. S buffer is further described in international application WO 2016/065341, e.g., at ¶ [0228]-[0229] and incorporated herein by reference in its entirety.

Post- the SOLUPORE ™ process and post-transduction
analyses completed for each experiment.

| | Quantification | Days Post- | | Run 2, 3 | |
|---|---|---|---|---|---|
| Analysis | Method | Infection | Run 1 | and 4 | Related SOP |
| Cell Count | Nucleocounter | 0 | ♦ | ♦ | “the |
| and Viability | NC-200 | 1 | ♦ | ♦ | SOLUPORE ™ |
| | | 3 | ♦ | ♦ | process of |
| | | 4 | | ♦ | PBMC Initiated |
| | | | | | T Cell Cultures” |
| | | | | | (described |
| | | | | | herein) |
| GFP | CytoFlex | 0 | ♦ | ♦ | |
| Expression | (Flow | 1 | ♦ | ♦ | |
| | Cytometry) | 3 | ♦ | ♦ | |
| | | 4 | | ♦ | |
| Integrated | ddPCR | 3 | | ♦ | |
| Copies of | (WPRE/Alb) | 4 | | ♦ | |
| GFP | | | | | |

♦ represents timepoint analysis tested.

For all post-the SOLUPORE™ process timepoints, samples were weighed, counted, and viability assessed by Nucleocounter NC-200 before collecting for downstream analyses.

For each run, complete media was doubled one day after viral delivery. Given the larger volumes for Run 1/2, the samples were transferred to a T75 flask and cultured lying flat, while for Run 3/4, 3 mL of complete media was simply added to each T25 flask. For Run 1, final analyses were performed three days after the SOLUPORE™ process. For Run 2 onwards, three-day post-the SOLUPORE™ process cultures were diluted to a VCD of 5×10⁵ cells/mL after timepoint analyses were performed to allow for further analysis on the next day. Post-dilution cell counts were carried out to track cumulative fold expansion up to four days after the SOLUPORE™ process. All post-recovery and post-the SOLUPORE™ process timepoint analyses were completed as listed above.

### Decontamination of Solupore™ System

Payload solution was removed from the Elveflow valve and was properly decontaminated before disposal. The atomiser was then rinsed by purging twice with 1 mL of PREempt RTU, twice with 1 mL of WFI, and twice with 1 mL of 70% IPA (isopropanol). Finally, the atomiser was purged again in the same sequence using 1 mL of each reagent. The system was disassembled and sprayed with PREempt RTU. 20 L of 1% v/v Citranox in tap water was prepared and system components were soaked and rinsed with DI (deionized) H₂O as described in. Each component was then sprayed with 70% IPA before being placed back into BSC and dried using an air gun. Gaskets and O-rings were placed in autoclavable bags and autoclaved on solid cycle. Ultraviolet light in BSC was activated to ensure all non-autoclavable components were sterilized. Ethylene Oxide (EtO) sterilisation steps were omitted.

### Preparation of Samples and Acquisition of 7-Aminoactinomycin D (7-AAD) Viability Staining and % GFP Data

For each timepoint, 200 μL of cells from each sample were collected in a 96-well V-bottom polypropylene plate. The cells were then pelleted by centrifugation at 300×g for 7 min and supernatant was removed using a multichannel pipette. During centrifugation, a master mix of 7-AAD viability dye in FACS solution (5 μL of 7-AAD in 200 μL FACS solution per sample) was prepared in a 15 mL Falcon tube. Each sample was resuspended in 200 μL of staining solution and incubated at RT for 5 min while protected from light. 7-AAD viability staining and GFP expression were acquired using CytoFlex and quantified using FlowJo according to the acquisition and gating strategy.

Droplet Digital Polymerase Chain Reaction (ddPCR): Sample Collection, DNA Extraction, and PCR Sample Preparation

For each timepoint analysis, 200 μL of cells from each sample was collected in a 96-well V-bottom polystyrene plate. The cells were then pelleted by centrifugation at 300×g for 7 min and supernatant was removed using a multichannel pipette. During centrifugation, 1× lysis buffer was prepared as follows:

Preparation of 1X In-House Cell Lysis Buffer.

| | Stock | Working |
|---|---|---|
| Reagent | Concentration | concentration |
| In-House Cell Lysis Buffer | 10X | 1X |
| Proteinase K | 1 mg/mL | 4 μg/mL |
| DNase-/RNase-free water | | For lysis buffer |
| | | dilution to 1X |

50 μL of 1× lysis buffer was added to each sample. All reactions were incubated at room temperature (RT) for 20 min before gently (to avoid foaming) transferring to a standard 96-well PCR plate. The reaction plate was sealed using a clear plastic film before incubating at 56° C. for 15 minutes, 95° C. for 10 minutes, and cooled to 4° C. using a thermocycler. Samples were then stored at −20° C. until additional samples from all experiments were collected.

To prepare samples for PCR, all DNA templates were diluted 20× using PCR Grade DNase-/RNase-free water as a diluent in a standard 96-well plate. 1× ddPCR super master mix was prepared in a 15 mL Falcon tube using the following compositions for each reagent/reaction:

Preparation of 1X ddPCR super master mix. Each reagent
volume was scaled up according to total number of samples.
A 10% volume contingency (for pipetting error)
and two extra reactions were also included as NTC controls.

| Reagent | Volume, μL/sample |
|---|---|
| 2X PCR mix | 11 |
| 20X primer/probe mix for WPRE (HEX) | 1.1 |
| 20X primer/probe mix for Alb (FAM) | 1.1 |
| PCR Grade DNase-/RNase-free water | 4.8 |
| Total | 18 |

18 μL of master mix was added to each reaction well of a 96-well Bio-Rad ddPCR plate in addition to 4 μL of 20× diluted DNA. For NTC controls, 4 μL of PCR Grade DNase-/RNase-free water was added. A pierceable foil heat seal was placed directly on top of the prepared plate and the plate was sealed using a plate sealer. The reactions were then vortexed and spun down briefly to collect loose liquid to the bottom before transferring to an automatic droplet generator (Bio-Rad AutoDG). Once droplets were generated, the plate was sealed again using a pierceable foil heat seal and transferred to the Bio-Rad C1000 thermocycler to initiate the PCR using the following cycle: 95° C. for 10 min (Ramp 2° C./sec), [94° C. for 30 sec (Ramp 2° C./sec), 60° C. for 1 min (Ramp 2° C./sec)]×39, 98° C. for 10 min and final hold at 4° C. Once the PCR cycle was completed, the reactions were transferred to the Bio-Rad QX200 droplet plate reader to quantify VCN by Alb/WPRE (Woodchuck Hepatitis Virus Post-transcriptional Regulatory Element) ratio. The position of the threshold line was determined using the analysis software, QuantaSoft, to yield a clear separation between the positive and negative populations in both the x and y directions. Based on a Poisson distribution, the concentration (in copies/μL) of both Alb and WPRE was obtained and % GFP was derived by applying the following equation:

$$\% G F P = \frac{WPRE copies / μL}{Albumin copies / μL \times 0.5 \frac{cells}{Albumin copy}}$$

### Software/Statistics

| Software | Description |
|---|---|
| Microsoft | General experimental planning, simple calculations |
| Excel | (e.g. average, standard deviations), graph generation, |
| | t-test, etc. |
| CytExpert | Interface for CytoFlex for acquisition of GFP, |
| | CD3 and CD25 expression by flow cytometry. |
| FlowJo | Analyse data generated from flow cytometry to determine |
| | GFP, CD3 and CD25 expression. |
| Minitab | Outlier analysis and ANOVAs. |
| QuantaSoft | Analyse data generated from ddPCR to determine |
| | integrated copies of GFP |

### LV Stability

Based on qualitative analysis using 4×, 10× and 20× magnification, there was no evidence of precipitation in delivery solution up to 4 hours after formulation.

### LV Delivery Run 1

CD3 and CD25 expression of activated PBMC-derived T-cells as determined by flow analysis was 92.3% and 91.3%, respectively.

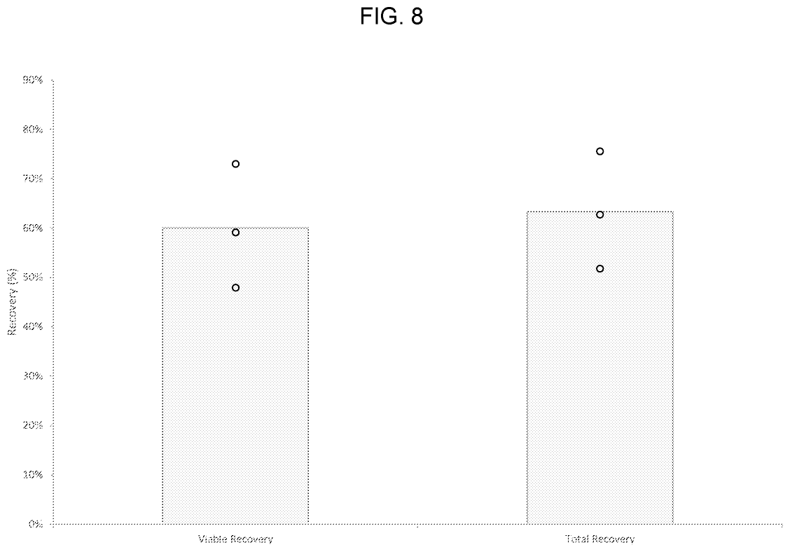

After the SOLUPORE™ process, the average total recovery from the SOLUPORE™ process was 63%±12% and viable recovery was 60%±13% for three replicates (FIG. 8).

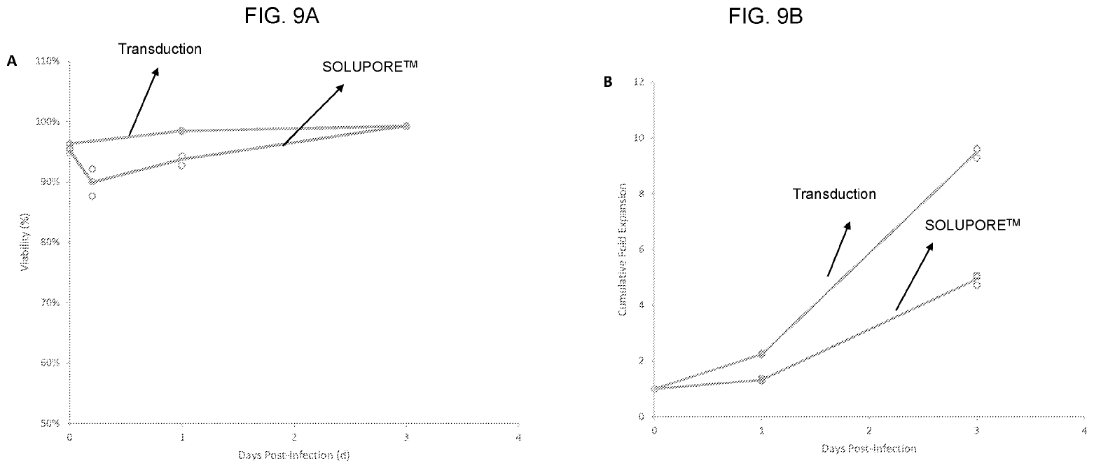

For both viral delivery methods, cell count and viability were assessed immediately after infection, as well as day 1, and day 3 post-infection. As shown in FIG. 9A, the viability of the soluporated cells one day after viral delivery was significantly lower (p<0.05) than statically transduced cells (98.5% and 93.8%, respectively). Nevertheless, the viability for soluporated and statically transduced cells was comparable 3 days post-infection (99.3% for both delivery methods).

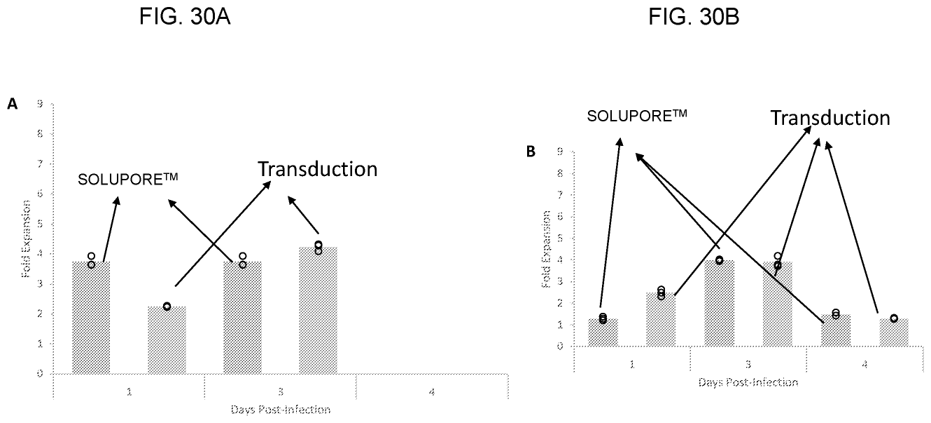

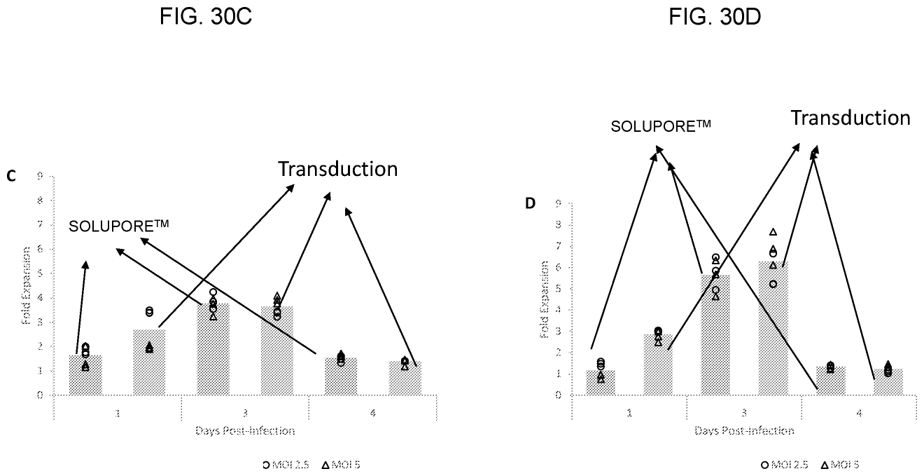

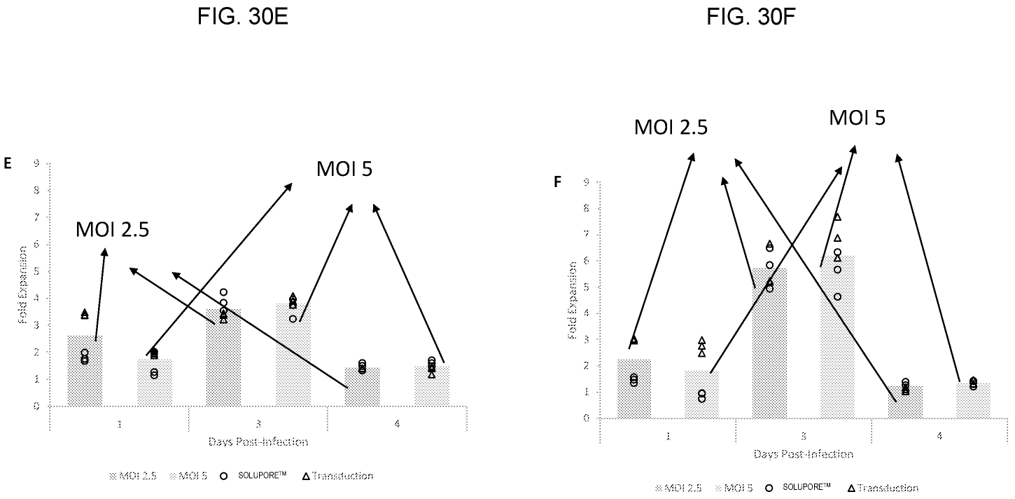

The cells harvested from the system post-the SOLUPORE™ process had a cumulative fold expansion of 4.9 after 3 days, This was significantly lower (p<0.01) than statically transduced cells which had a cumulative fold expansion of 9.5 (FIG. 9B). A significant difference in fold-expansion was observed on day 1, as seen in FIG. 30A.

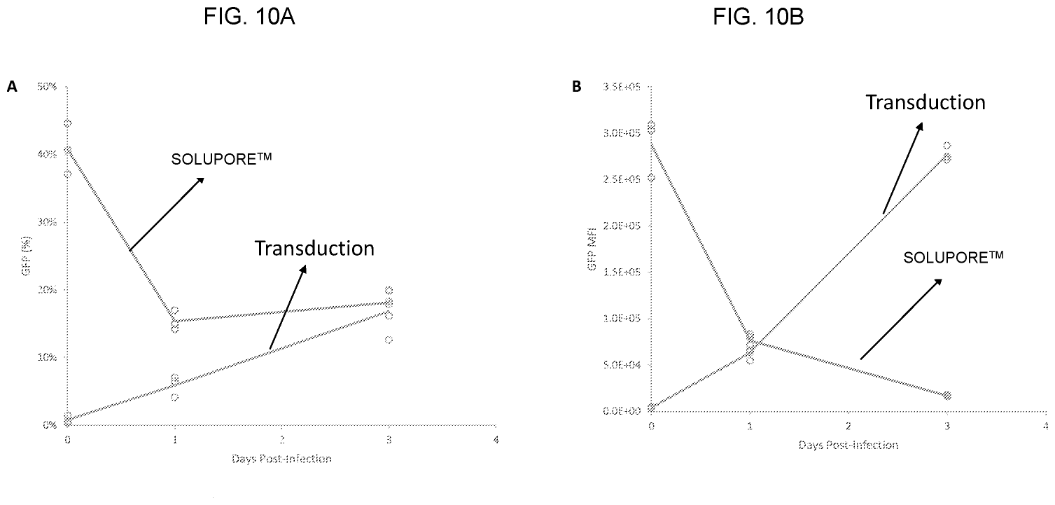

To assess viral delivery efficiencies, GFP expression and median fluorescence intensity were quantified by flow cytometry. The % GFP expression after 3 days was 18.1% for soluporated T-cells and 16.8% for statically transduced T-cells (FIG. 10A). The soluporated T-cells had a significantly higher % GFP expression (p<0.01) up to one day after viral delivery.

The MFI of the expressed GFP was significantly higher (p<0.01) for the soluporated T-cells immediately after viral delivery (FIG. 10B). However, this decreased over time, and after 3 days of culture, the soluporated T-cells exhibited a significantly lower MFI than statically transduced T-cells (p<0.001).

### LV Delivery Run 2

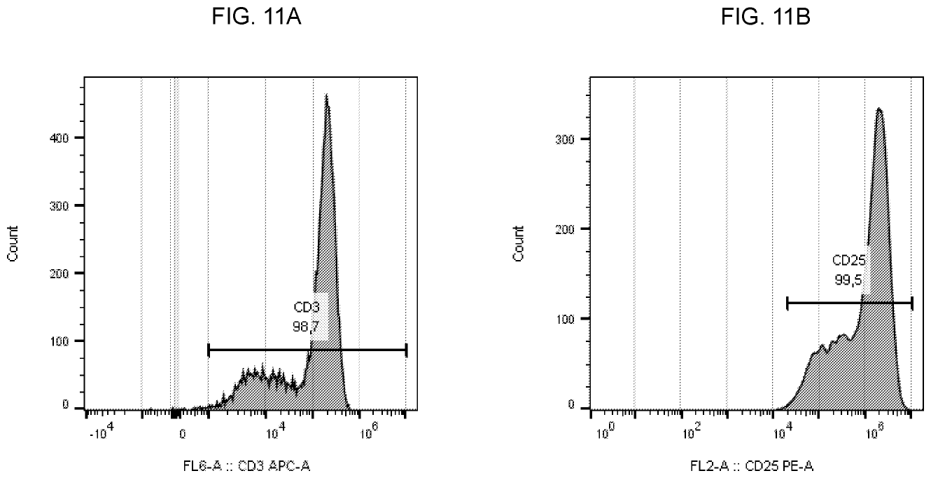

The second run of viral delivery by both the SOLUPORE™ process with the system and static transduction was performed similarly to the first run. After 3 days of activation from PBMCs, the cell population before delivery was 96.7% CD3⁺ and 99.5% CD3⁺CD25⁺ (FIGS. 11A and 11B).

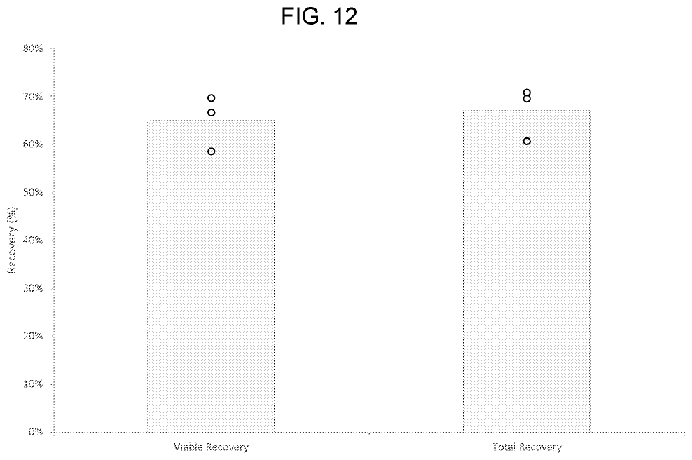

After viral delivery by the SOLUPORE™ process, the average total cell recovery was 67%±6% and viable recovery was 65%±6% for three technical replicates (FIG. 12).

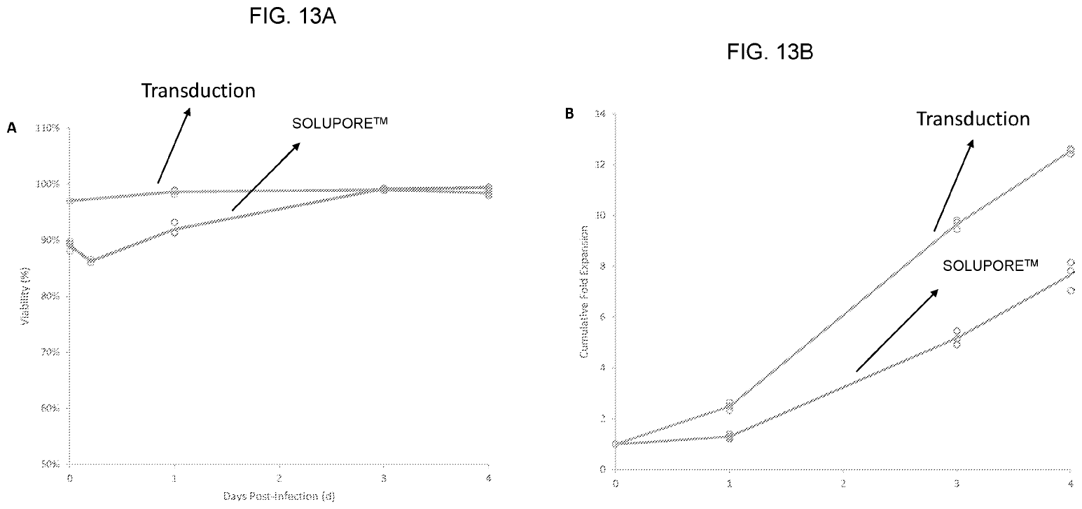

As illustrated 4-day post-infection analyses were performed for this run to validate the GFP expression observed in earlier timepoints. Although cell viability was >90% for both viral delivery methods one day post-infection (FIG. 13A), the viability of soluporated cells was significantly lower (p<0.01) than statically transduced cells (92.0% and 98.6%, respectively). In later timepoints, soluporated cells recovered and no significant difference in viability between the two delivery methods was observed.

The cells harvested from the system post-the SOLUPORE™ process had a cumulative fold expansion of 7.7 after 4 days. This was significantly lower (p<0.01) than statically transduced cells which had a cumulative fold expansion of 12.5 (FIG. 13B). In addition, the fold expansion at each post-delivery timepoint was significantly lower (p<0.01) in the soluporated cells when compared to the statically transduced cells (FIG. 30B).

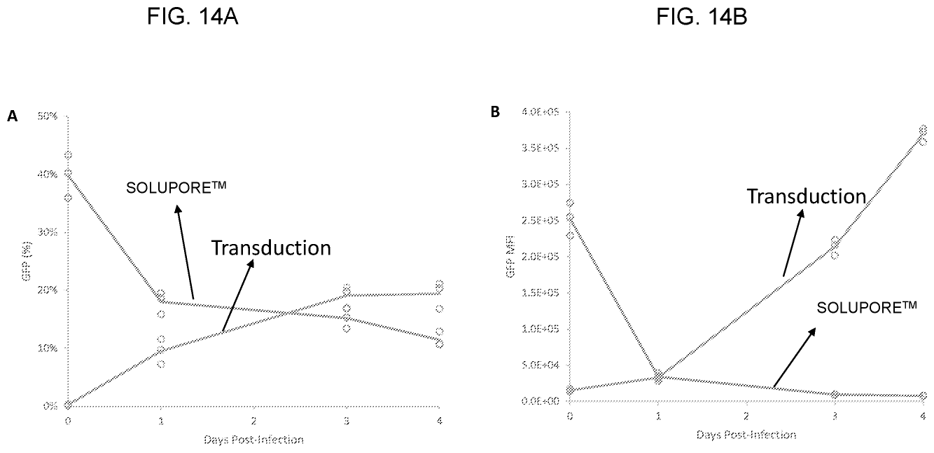

As shown in FIG. 14A, % GFP expression as quantified by flow cytometry 3 days post-infection was 15.2% for soluporated T-cells and 19.1% for statically transduced T-cells. After an additional day, the % GFP expression was maintained for the statically transduced cells (19.4%), but the GFP expression in the soluporated cells significantly decreased to 11.5% (p<0.05). The soluporated T-cells had a significantly higher % GFP expression (p<0.01) up to one day after viral delivery.

The MFI of the expressed GFP was significantly higher (p<0.01) for the soluporated T-cells immediately after viral delivery (FIG. 14B). However, the MFI in soluporated T-cells decreased over time. After 3 and 4 days of culture, the T-cells from static transductions had a significantly higher MFI (p<0.001).

### LV Delivery Run 3

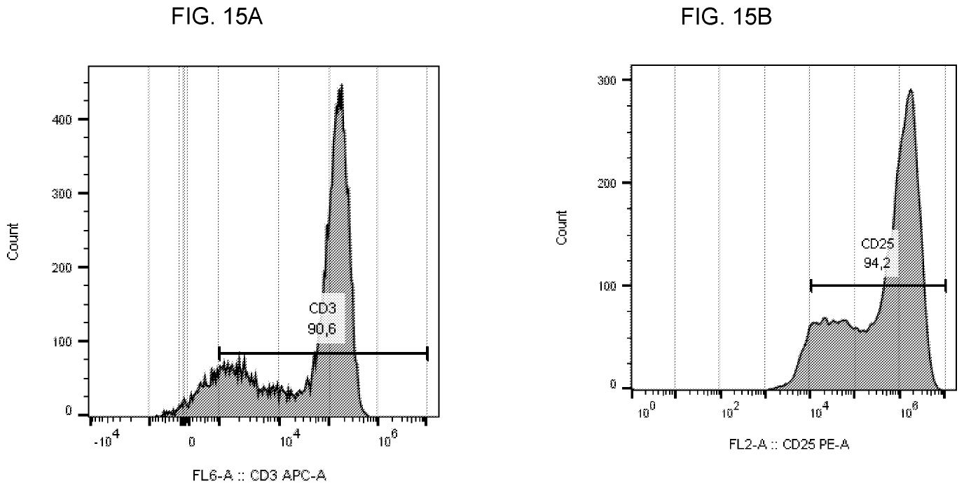

After 3 days of activation from PBMCs, the cell population before delivery was 90.6% CD3+ and 94.2% CD3+CD25+(FIGS. 15A and 15B).

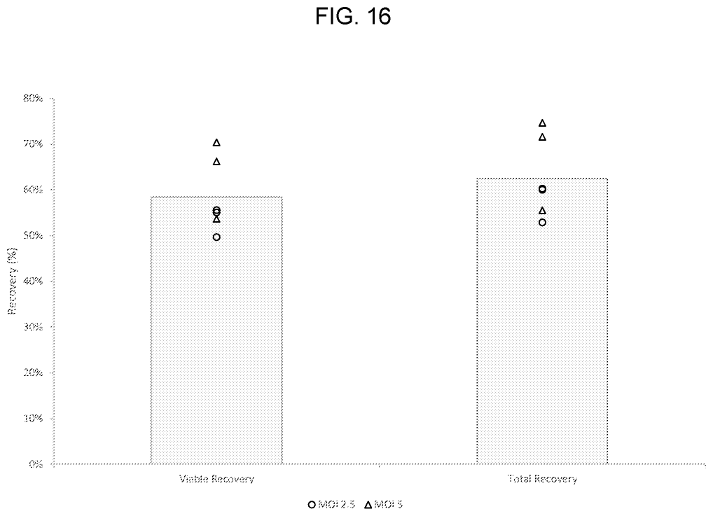

Immediately after the SOLUPORE™ process, the average total recovery was 62%±9% and viable recovery was 58%±8% (FIG. 16). There was no significant difference in recovery despite the difference in number of cells loaded (p>0.05; paired two-tailed t-test).

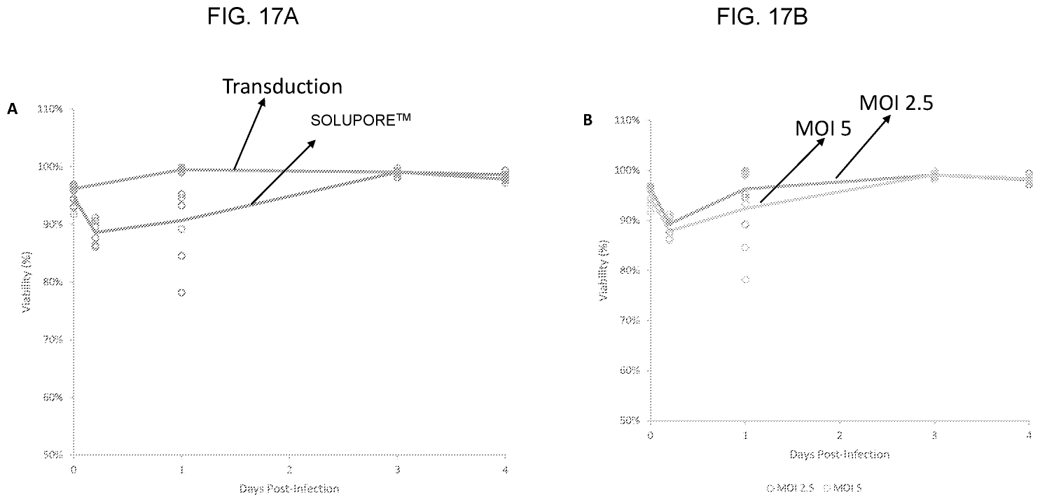

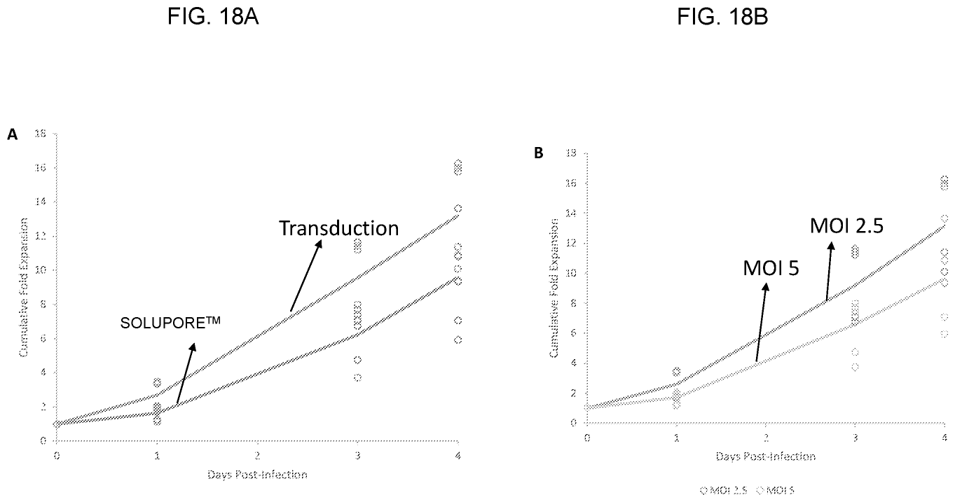

As illustrated in FIG. 17A, the viability of the soluporated cells after one day was significantly lower (p<0.01) than the viability of the cells that underwent viral delivery by static transduction (90.7% and 99.5%, respectively). In later timepoints, the viability of soluporated cells recovered to >99%, and was significantly higher (p<0.05) than the viability of statically transduced cells (98.7% and 97.9%, respectively) on day 4. These findings were independent of MOI used, which appeared to have no effect on viability (FIG. 17B).

The cells harvested from the system post-the SOLUPORE™ process had a significantly lower (p<0.05) cumulative fold expansion of 9.6 after 4 days than the cells from static transduction, which had a cumulative fold expansion of 13.2 (FIG. 13A). Regardless of method of viral delivery, cells transduced with a lower MOI of 2.5 had significantly better expansion (p<0.05) over 4 days (FIG. 13B).

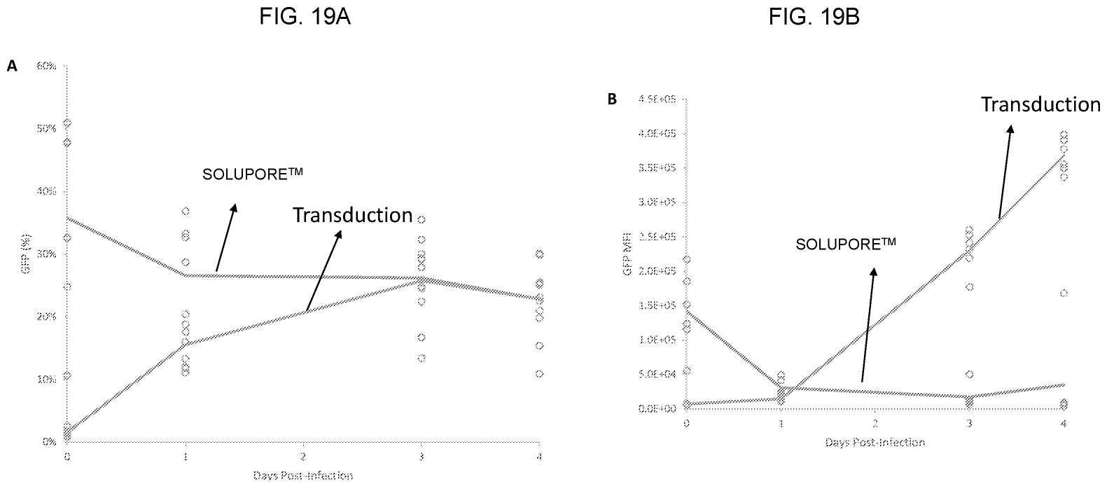

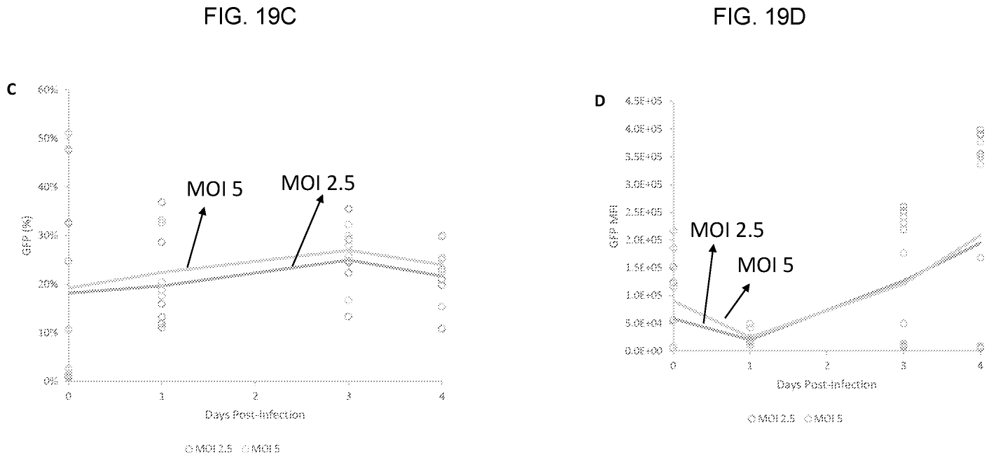

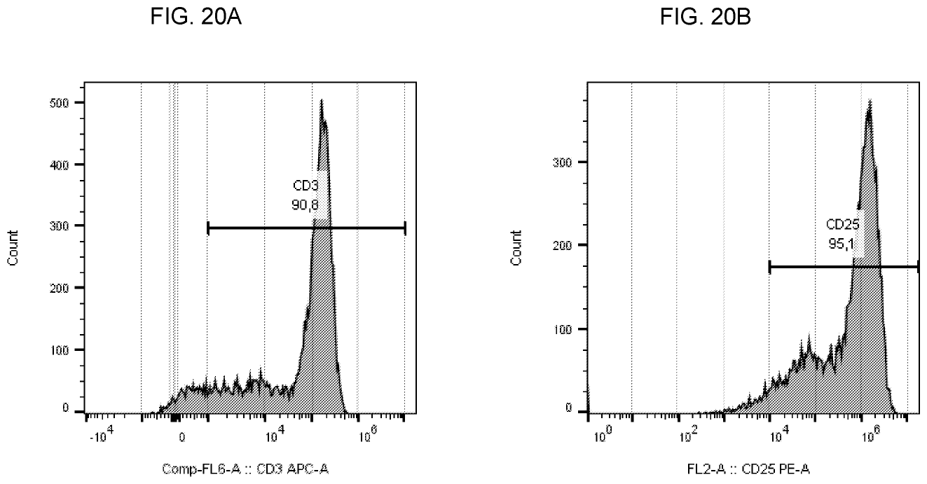

Representative samples were taken at each timepoint post-infection to evaluate efficiency of viral delivery in both delivery methods. As quantified by flow cytometry, % GFP after 3 days was 26.2% for soluporated T-cells and 25.8% for statically transduced T-cells (FIG. 19A). After an additional day, % GFP decreased (p<0.01) for both the SOLUPORE™ process and static transduction to 22.8% and 22.9%, respectively. The soluporated T-cells had a significantly higher % GFP (p<0.01) up to one day after viral delivery. There was no statistical difference in % GFP between a MOI of 2.5 or 5, regardless of LV delivery method. (FIG. 19C).

The Median Fluorescence Intensity (MFI) of the expressed GFP was significantly higher (p<0.001) for the soluporated T-cells immediately after viral delivery (FIG. 19A-19D and FIG. 10B). However, the MFI of the soluporated T-cells decreased over time, and after 3 and 4 days of culture, the T-cells from static transduction had a significantly higher MFI (p<0.001). There was no statistical difference in GFP MFI between a Multiplicity of Infection (MOI) of 2.5 or 5, regardless of LV delivery method (FIG. 19D).

### LV Delivery Run 4

The fourth run of viral delivery by both the SOLUPORE™ process with the system and static transduction was conducted similarly to the third run. After 3 days of activation from PBMCs, the cell population before delivery was 90.8% CD3+ and 95.1% CD3+CD25+(FIGS. 20A and 20B).

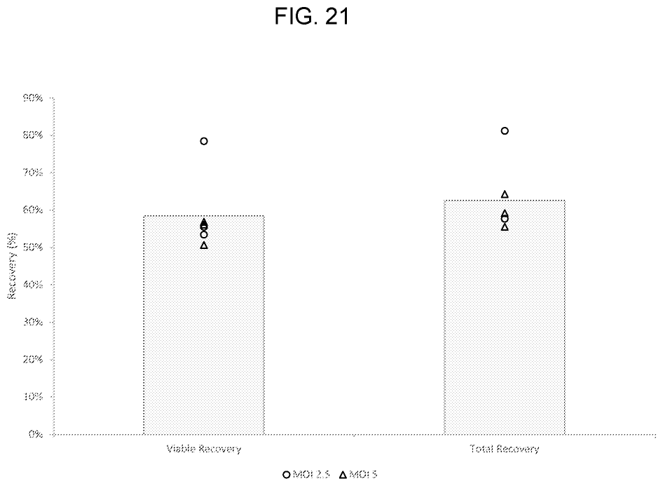

After viral delivery by the SOLUPORE™ process, the average total cell recovery was 63%±10% and viable recovery was 59%±10% for six replicates (FIG. 21). There was no significant difference in recovery despite the difference in number of cells loaded (p>0.05; paired two-tailed t-test).

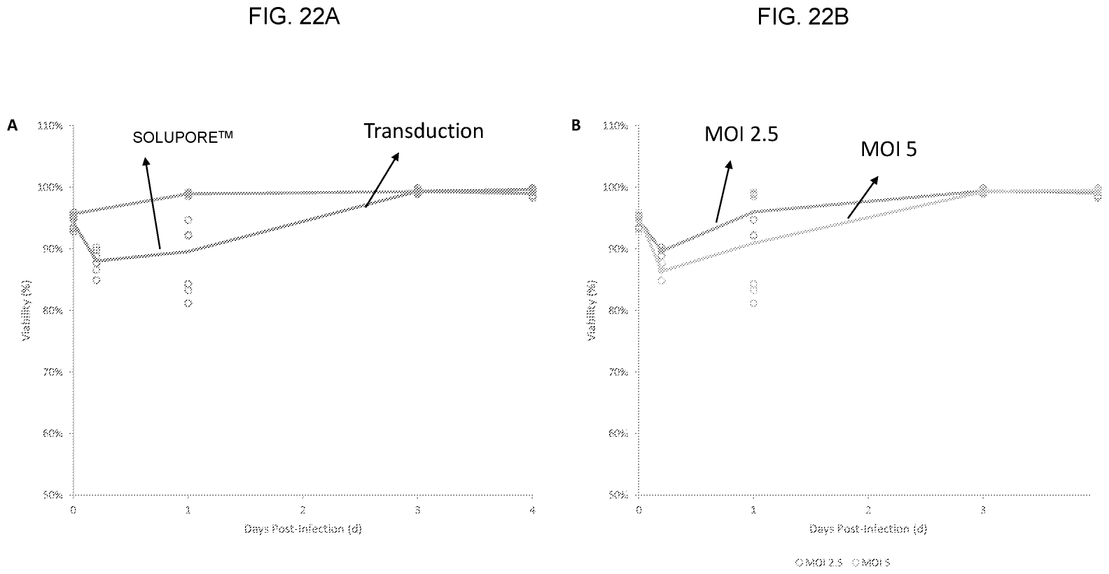

The viability of statically transduced cells in all post-infection timepoints was >98%. In comparison, the viability of soluporated cells was significantly lower (p<0.001) than statically transduced cells on day 1 post-infection (89.6% and 99% respectively, FIG. 22A). In later timepoints, the viability of soluporated cells recovered to >99%, and was significantly higher (p<0.05) than the viability of statically transduced cells (99.7% and 99.0%, respectively) on day 4. Regardless of the method of viral delivery, cells transduced with a lower MOI of 2.5 had significantly better viability (p<0.05) one day after infection (FIG. 22B). In later timepoints, the viability of cells of both MOIs had comparable values (>98%), and the higher MOI of 5 had significantly higher (p<0.05) viability on day 4 compared to the lower MOI of 2.5 (99.6% and 99.1%, respectively).

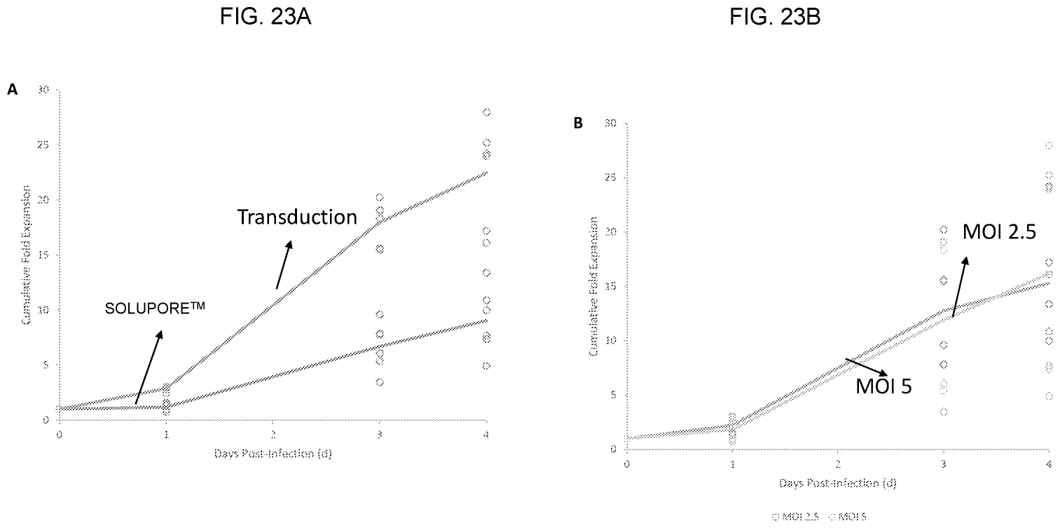

The cells harvested from the system post-the SOLUPORE™ process had a significantly lower (p<0.001) cumulative fold expansion of 9.0 after 4 days than the cells from static transduction, which had a cumulative fold expansion of 22.5 (FIG. 23A). There was no statistical difference in expansion between a MOI of 2.5 or 5, regardless of the method of viral delivery (FIG. 23B).

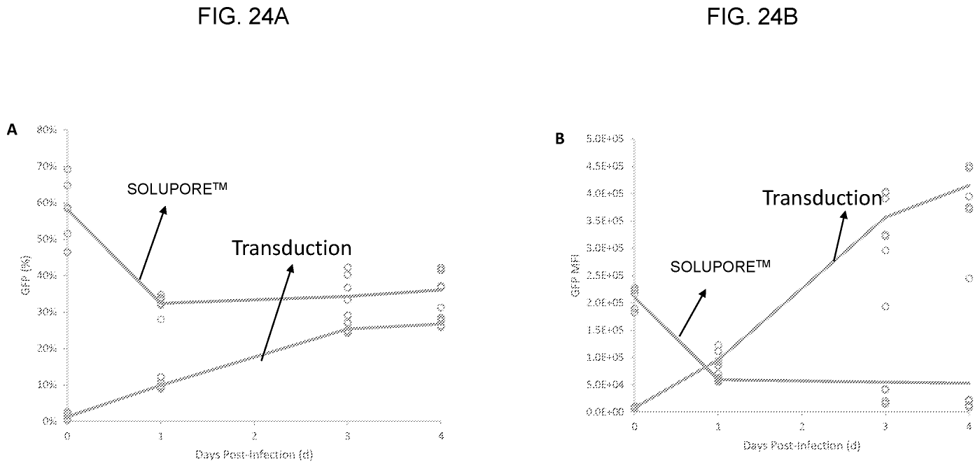

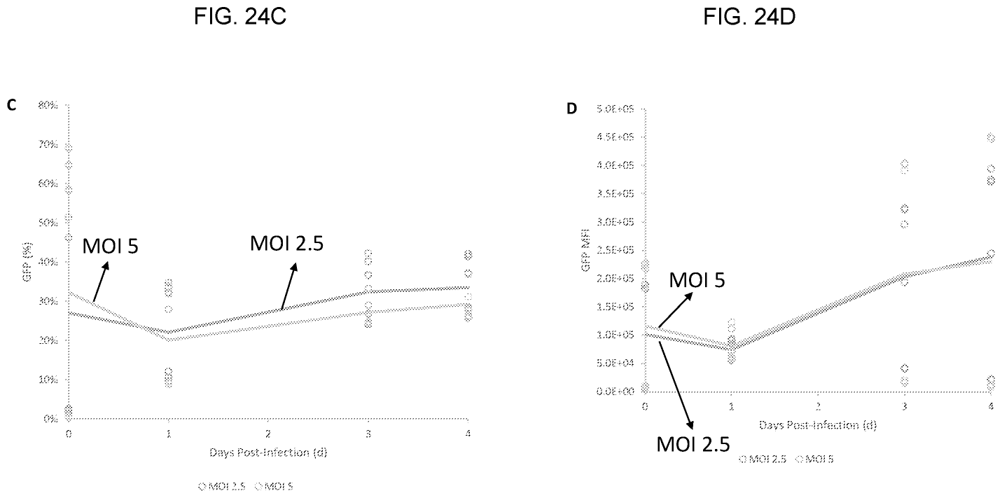

3 days after delivery, expression of GFP in % was significantly higher (p<0.01) in soluporated T-cells (34.3%) than statically transduced cells (25.4%) (FIG. 24A). After an additional day, the GFP expression was maintained for both the SOLUPORE™ process and static transduction at 36.1% and 26.7%, respectively. As shown in FIG. 24C, method of viral delivery and MOI conditions had no significant impact on % GFP expression.

The Median Fluorescence Intensity (MFI) of the expressed GFP was significantly higher (p<0.001) for the soluporated T-cells immediately after viral delivery (FIG. 24B). However, the MFI of the soluporated T-cells decreased over time, and after 3 days of culture, MFI was significantly lower than statically transduced T-cells (p<0.001). Similarly, despite method of viral delivery and MOI used, there was no statistical difference in GFP MFI (FIG. 24D).

### T-Cell Purity and Activation

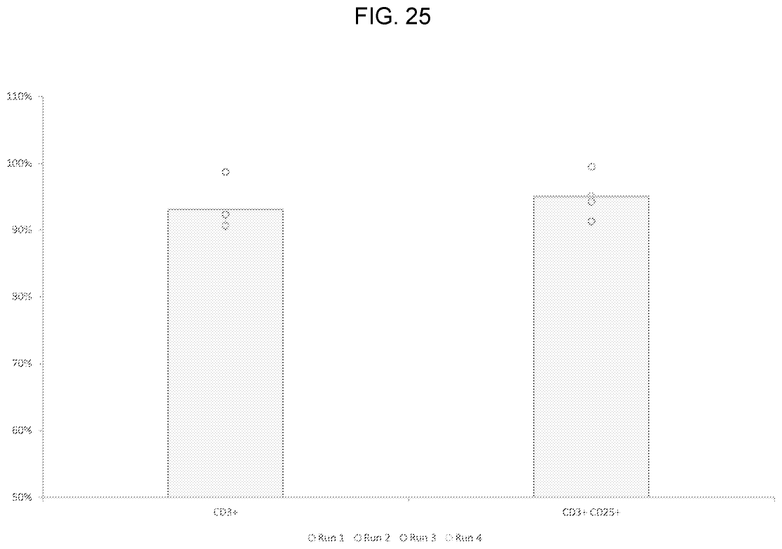

To meet release criterion for experimental use, PBMC-initiated T-cells were required to have >90% expression for both CD3 and CD3/CD25. In all runs, the cells passed QC with an average of 93.1%±4% CD3+ and 95.0%±3% CD3+CD25+ and were released for the SOLUPORE™ process (FIG. 25).

### Post-Recovery Analysis

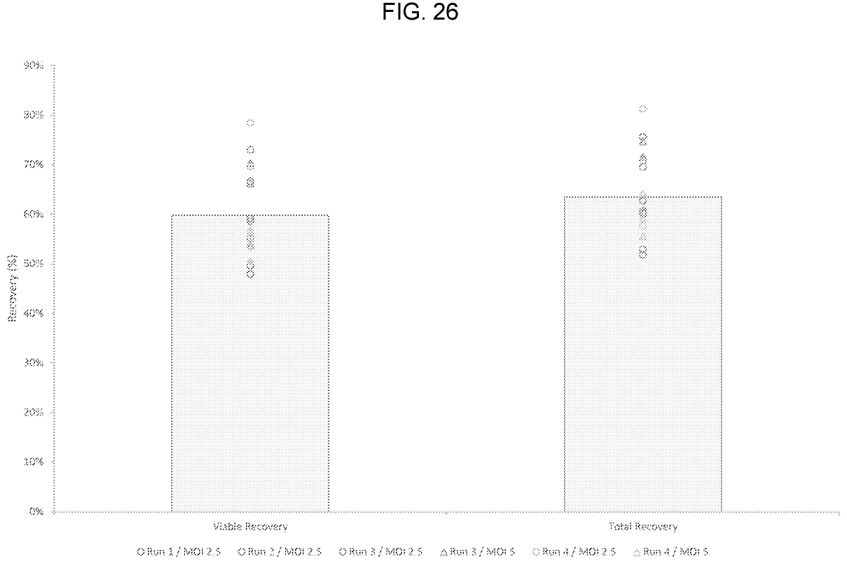

During the technology transfer of the system to the facility, a minimal threshold of ≥50%±10% total cell recovery and ≥30%±10% viable cell recovery was used to define a successful the SOLUPORE™ process run. Cumulatively, the total cell recovery was 63%±9% and the viable cell recovery was 60%±9% (n=18 sprays, FIG. 26). Although different cell loads of 24×10⁶ and 12×10⁶ cells were tested for the third and fourth run, there was no significant difference (p>0.05; paired two-tailed t-test) in the recovery percentage between the two cell loading conditions.

Viability after Viral Delivery

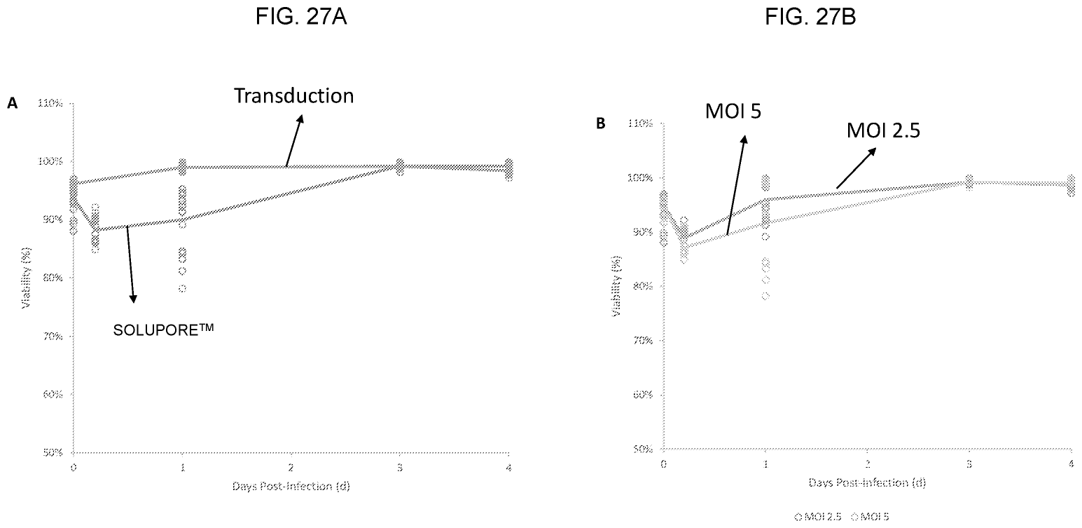

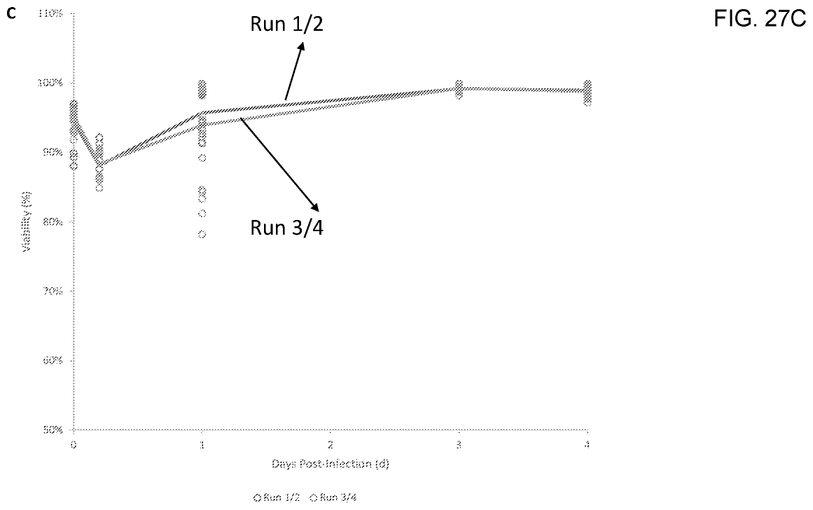

An additional criterion for a successful the SOLUPORE™ process was ≥70%±10% viability one day post-the SOLUPORE™ process. The average viability of all 4 runs one day post-the SOLUPORE™ process was 90.0%±5.3% (n=18 sprays). However, this was lower (p<0.001) than the viability of statically transduced T-cells at day one (99.0%, FIG. 27A). The lower viability noted one day post-infection may be due to the decrease in viability typically seen immediately post SOLUPORE™ process (88.3%; n=18 sprays), e.g., a recovery period. Despite this observation, there was no significant difference in viability on day 3 when compared to statically transduced cells (>98% for both viral delivery methods). This demonstrates that soluporated T-cells are able to recover over time. Furthermore, at 4 days post-the SOLUPORE™ process, soluporated cells had a significantly higher (p<0.01) cell viability (99.2%) compared to statically transduced cells (98.4%).

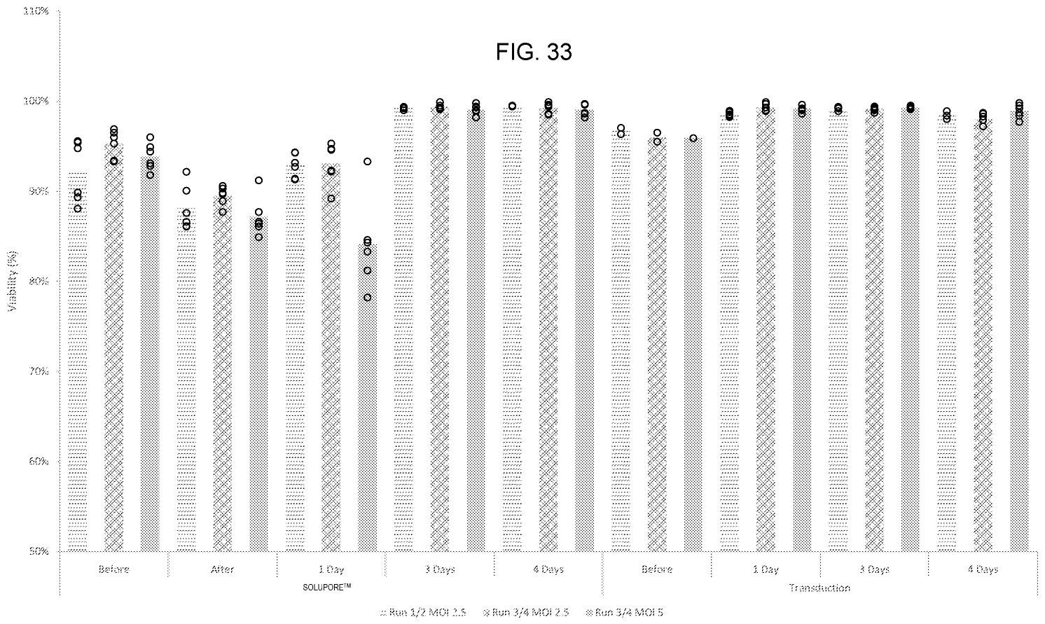

Independent of the method used for viral delivery, a lower MOI of 2.5 resulted in a significantly higher (p<0.01) viability 1 day post-infection when compared to MOI of 5 (96.0% and 91.7% respectively, FIG. 27B). This is observed in cells that underwent the SOLUPORE™ process and not for static transduction (FIG. 33). Despite the changes made to calibration and post-infection culturing conditions, there were no statistical differences in viability between Runs 1/2 and Runs 3/4 (FIG. 27C).

Expansion Kinetics after Viral Delivery

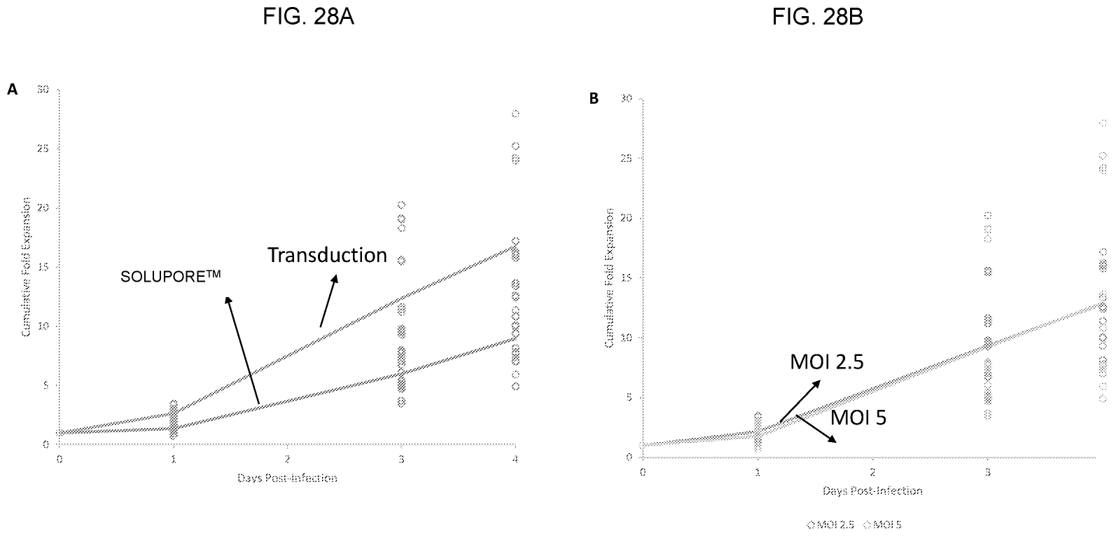

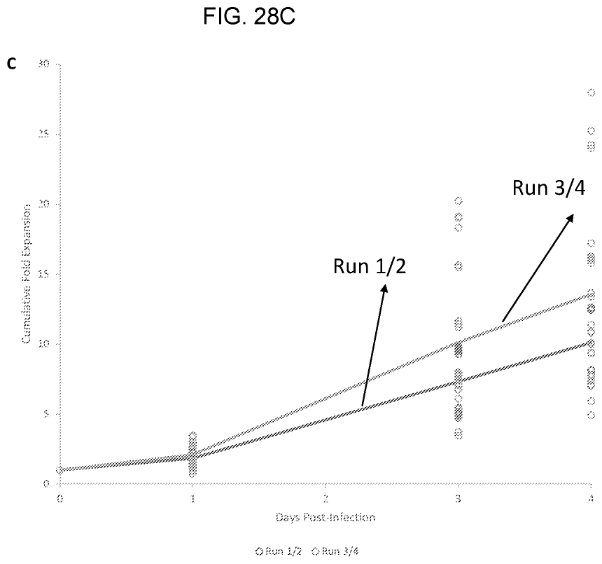

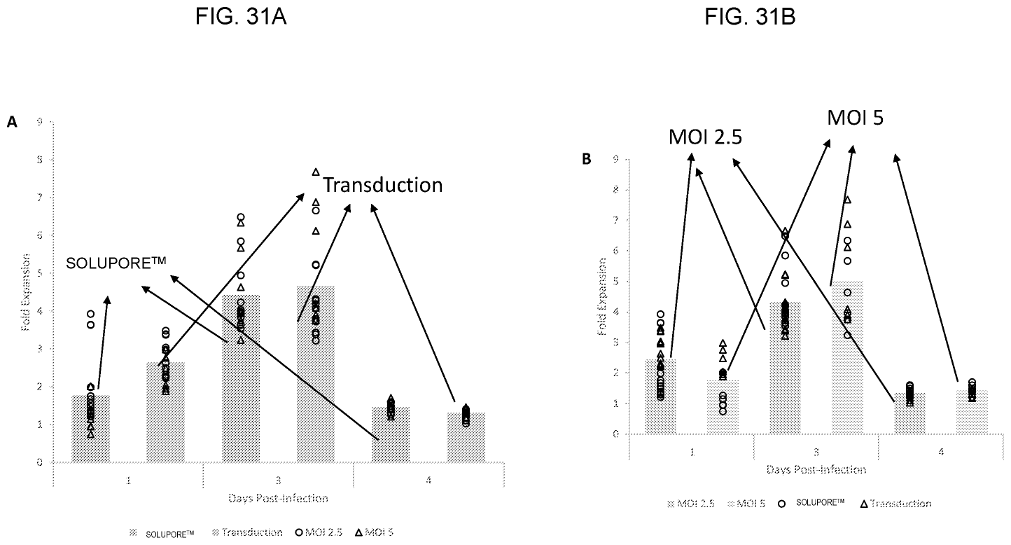

After viral delivery by either the SOLUPORE™ process or static transduction, cell counts were performed up to 4 days post-infection to assess the impact of viral delivery on T-cell proliferation. After 4 days, an average fold expansion of 16.8 was observed in statically transduced T-cells. This was significantly higher (p<0.001) than soluporated T-cells, which experienced a 9.0 fold expansion (FIG. 28A). The difference in the cumulative fold expansion observed in all four runs is primarily due to the recovery period required by the cells after the SOLUPORE™ process, which is compounded further at later days due to the multiplicative nature of cumulative fold expansion. Daily-fold expansion is shown in FIGS. 31A and 31B.

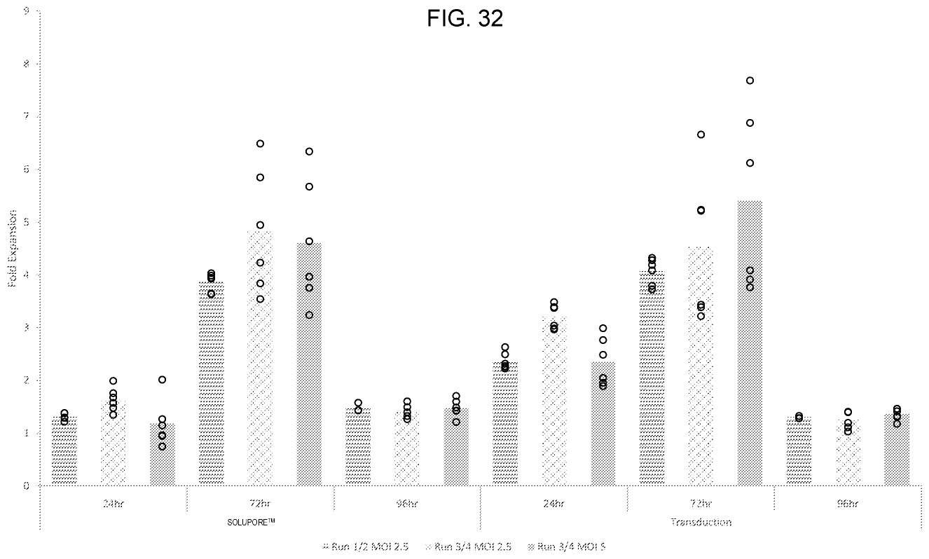

In addition, one day after delivery, T-cells that received the lower MOI of 2.5 had higher (p<0.001) expansion than those that received MOI of 5 regardless of delivery method (FIG. 28B). However, this difference was not observed at 3 or 4 days post-delivery. Again, this was primarily observed in the soluporated cells and not in the statically transduced cells (FIG. 32) and may be similarly related to the lower viability observed post SOLUPORE™ process (recovery period).

As shown in FIG. 28C, Runs 3 and 4 had significantly higher expansion compared to Runs 1 and 2 at one day (p<0.001) and three day (p<0.01) post-delivery. However, this effect was not observed at 4 day post-delivery. The differences observed in expansion kinetics may be due to the alterations made to cell culturing techniques in Runs 3 and 4, specifically the volume-to-surface area ratio. A reduction in volume-to-surface ratio in Runs 3 and 4 possibly allowed for proper oxygen diffusion throughout cell cultures, thereby encouraging cell proliferation. This reduced volume-to-surface area ratio was achieved for the whole duration of culture beginning immediately after the SOLUPORE™ process in Runs 3 and 4. For Runs 1 and 2, the volume-to-surface area ratio was reduced after one day post-the SOLUPORE™ process upon transfer of the cultures from upright T25 flasks to T75 flasks laying flat. The data at Day 4 suggests that these changes may not have persistent long-term effects on T-cell expansion.

### GFP Expression by Flow Cytometry

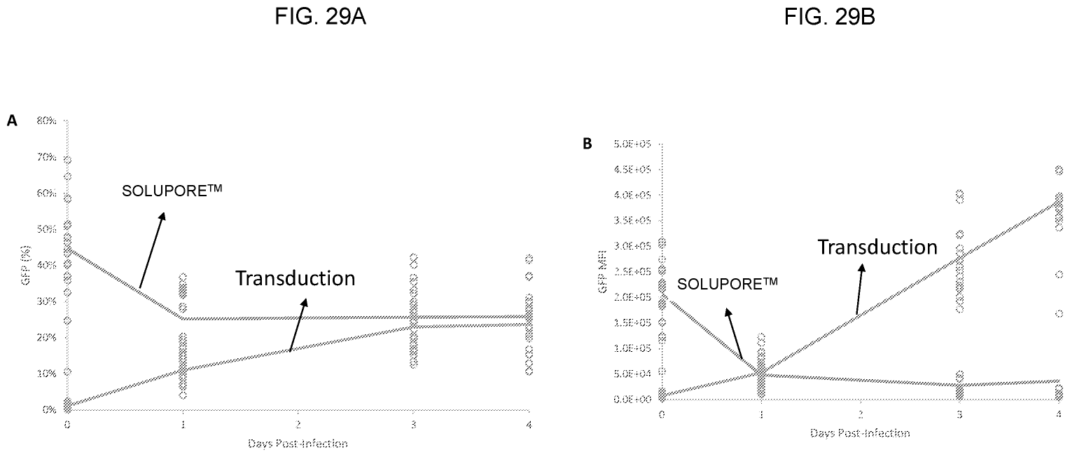

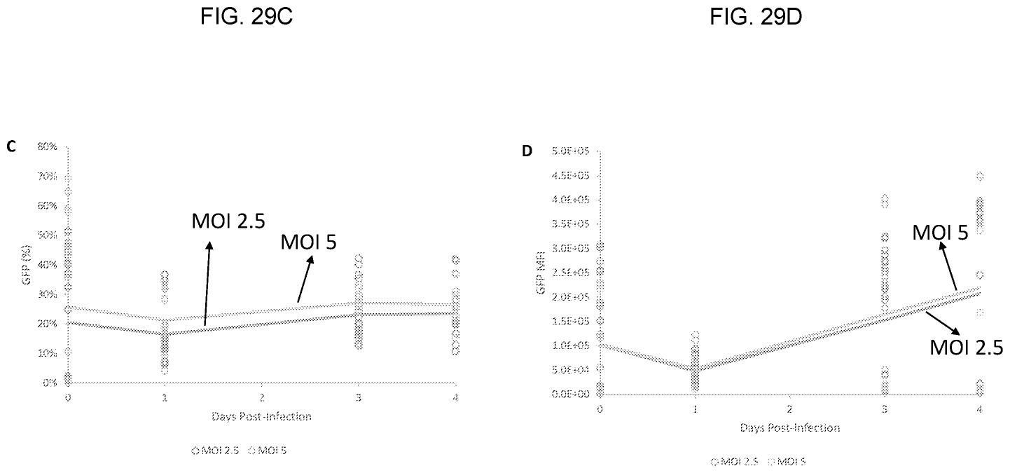

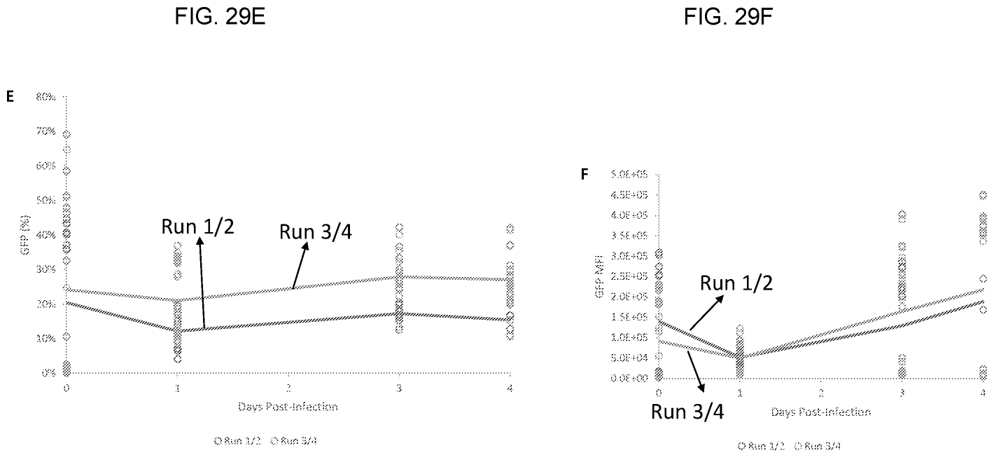

In all runs, the GFP expression immediately post-the SOLUPORE™ process was significantly higher (p<0.001) than in statically transduced T-cells (44.7% and 1.1%, respectively, FIG. 29A). This significant difference was also observed one day post-delivery (p<0.001). Considering the LV-eGFP used for this work package was a crude virus prep, this difference in GFP expression at earlier timepoints can be explained by the fact that the cells likely received GFP as protein cargo from the reversible permeability of the cell membrane during the SOLUPORE™ process, whereas statically transduced cells were not subjected to this procedure. As the data suggests (FIG. 29A), this difference decreases over time as the protein cargo is only transiently present and is diluted out as the cells expand. In order to address the possibility of protein cargo delivery, an additional timepoint at 4-day post-the SOLUPORE™ process was introduced for Run 2 onwards to assess whether % GFP observed at the 3-day timepoint was an accurate depiction of successful LV delivery as opposed to residual GFP protein uptake during the SOLUPORE™ process. Given that in Runs 2, 3 and 4, the average GFP % 3 and 4 days post-the SOLUPORE™ process was 25.7% and 25.9% with no difference (p>0.05; paired two-tailed t-test) between the two timepoints, this suggests the results gathered on day 3 post-the SOLUPORE™ process are an accurate assessment of LV delivery efficiency.

Independent of viral delivery method, there were no significant differences observed in GFP % or MFI based on MOI (FIG. 29C and FIG. 29D). This is in line with what was observed in historical data generated and further validates the suggestion of using MOI of 2.5.

### Summary

Viable cell recovery and total cell recovery post-the SOLUPORE™ process across 18 samples was 60% and 63%, respectively.
Viability 24 hours post SOLUPORE™ process was 90% (n=18 samples).
The system is capable of delivering LV-eGFP to T-cells with an average GFP expression of 25.7% across 18 the SOLUPORE™ process experiments.

Altering calibration to dispense appropriate payload solution volume as well as optimizing cell culture parameters (i.e. volume-to-surface area ratio) significantly improved GFP expression.

### Example 6: Isolation, Initiation and Cell Culture of PBMC Derived T Cells

Isolation, Initiation and cell culture of PBMC derived T cells The method herein describes the isolation, initiation, and cell culture of T cells from PBMCs. The exemplary method outlined below covers T cells initiated from PBMCs which includes the isolation, culture and initiation of these cells.

### Relevant Acronyms

AB serum: Human serum from type AB donors who lack antibodies against the A and B blood-type antigens.

### APC: Allophycocyanin

### BSC: Biological Safety Cabinet

### DMSO: Dimethyl Sulphoxide

### DPBS: Dulbecco's Phosphate Buffered Saline

IL-2: Interleukin 2 is a cytokine required for T cell growth and survival.
IU: international units

### FBS-HI: Fetal Bovine Serum-Heat Inactivated

mRNA: messenger RNA
PBMC: peripheral blood mononuclear cells
RT: room temperature
TCGM: T cell growth medium

### 7-AAD: 7-Aminoactinomycin D

### Materials

| | | Product |
|---|---|---|
| Name | Supplier | Number |
| Lymphoprep | Stem Cell Technologies | 07811 |
| Heat-inactivated FBS | Gibco (Bio Sciences) | 10500064 |
| CTS OptMizer | Thermo Fisher Scientific | A1048501 |
| Human AB serum | Valley Biomedical | HP1022 |
| L-glutamine 200 mM (100X) | Gibco | 25030081 |
| GlutaMAX Supplement | Gibco | 350-50-061 |
| 200 mM (100X) | | |
| HEPES (1 M) | Gibco | 15630-080 |
| PBMC | Approved validated source | n/a |
| Recombinant Human IL-2 | CellGenix | 001420-050 |
| Human anti-CD3 (100 μg/ml) | Miltenyi | 130-093-387 |
| Human anti-CD28 (100 μg/ml) | Miltenyi | 130-093-375 |
| DPBS | Thermo Fisher | 14190094 |
| terilin 30 ml containers | Fisher Scientific | 14190094 |

### Equipment

| Equipment Name | Asset ID |
|---|---|
| Eppendorf Centrifuge, 5804 | AV-323-38-1 |
| Eppendorf 5424R Centrifuge (Small) | ID_000005 |
| Lonza 4D-Nucleofector | ID_000222 |
| BD Accuri C6 Flow cytometer | ID_000047 |
| Eppendorf Galaxy 170S Incubator | ID_000003 |
| Chemometec Nucleocounter | AV-323-36-1 |
| Controlled Rate Freezer | AV-320-18-1 |

### Procedure

### PBMC Isolation

Preparing dilution buffer:

| | |
|---|---|
| Dilution Buffer 500 ml | Dilution Buffer 500 ml |
| DPBS (1X) 495 ml | DPBS (1X) 495 ml |
| FBS-HI5 ml | FBS-HI 5 ml |

Preparing CTS T cell Growth Media (TCGM)

| | | |
|---|---|---|
| T Cell Growth Media 50 ml | 50 | ml |
| CTS OpTmizer T Cell Expansion SFM (Complete with | 46.3 | ml |
| OpTmizer ™ T-Cell Expansion Supplement) | | |
| Human AB serum (5%) | 2.5 | ml |
| L-glutamine (2 mM) | 500 | μl |
| IL-2 (250 IU/ml)* | 60 | μl |

*On day 0 add IL-2. Once IL-2 is added use within one week - always make up fresh on day of initiation.
1. Remember to immediately add CTS Supplement upon opening bottle of CTS.
2. Add serum or equivalent and filter using a 50 ml syringe and 0.2 μm filters or using a 0.2 μm SteriCup.
3. Once filtered, add L-glutamine and IL-2.
4. Use media within one week.

### Isolation of PBMCs

1. Dilute blood with DPBS containing 1% heat-inactivated FBS (named Dilution Buffer) in a sterile Duran.
1.1. Dilute Buffy coat in a 1:1 ratio (Blood:Dilution Buffer).
1.2. Dilute Leukopak contents in a 1:2 ratio (Blood:Dilution Buffer).

2. Add 10 ml of Lymphoprep solution to 50 ml Falcons (number of Falcons is number of mls diluted blood total divided by 40).

3. Carefully layer diluted blood onto prepared Lymphoprep by holding the 50 ml tube perpendicular to the 25 ml stripette.

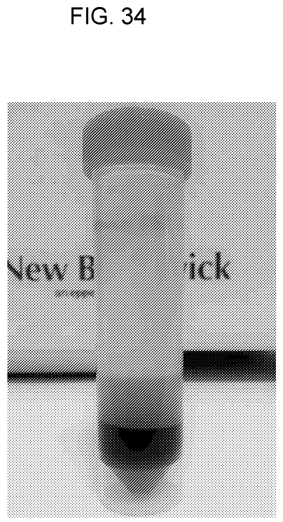

4. Centrifuge for 22 min at 400 g with the acceleration set to 6 and brake off (set to 0—minimum deceleration). See FIG. 34.

5. Remove the top (serum) layer using a sterile pasteur pipette and discard, leaving 3 mls of your serum layer in your tube.

6. Carefully transfer the PBMC layer (white cloudy layer that sits under the serum and on top of the Lymphoprep) to a clean tube. Ensure that none of the red cell pellet is taken.

7. Top up the buffy coat layers to 50 ml with DPBS+1% HI-FBS, mix and centrifuge for 7 min at 450×g with the brake on (acceleration 9, deceleration 9—leave at this setting for the remainder of the protocol).

8. Pour off the supernatant taking care not to disturb the PBMC pellet. Resuspend the pellet in a small volume (<1 ml or the amount of liquid remaining). Vortex.

9. Top up the PBMCs to 50 ml with DPBS+1% HI-FBS, mix and centrifuge 8.9. for 7 min at 450 g.

10. Discard the supernatant by pouring off. Resuspend the pellet in a 30 ml DPBS+1% HI-FBS. This is the isolated PBMCs.

11. Count cells: In an eppendorf, add 50 μl of cell suspension to 950 μl of DPBS+1% HI-FBS to make a 1:20 dilution. Using a Vial-Casette, take up diluted cell suspension and add to Nucleocounter. Count cells under “Cell Count and Viability” program, ensuring to add cell dilution into the program. See WI-6 NC-3000 NucleoCounter Operation & Maintenance.

12. PBMC can be cryopreserved at this point by following the freezing steps below in the ‘Freezing’ section. PBMC's should be frozen at 50 million cells per ml.

### Freezing PBMCs

1. To cryopreserve PBMCs, centrifuge the cell suspension at 400 g for 5 mins and remove the supernatant.

2 Prepare freeze media as 90% HI-FBS+10% dimethyl sulphoxide (DMSO) and store on ice. Make 1 ml of freeze media for every 50 million cells.

3. Cells can be frozen down using Mr. Frosty's or the controlled-rate freezer.

Mr. Frosty

1. Mr. Frosty's should be topped up to the mark on the container with room temperature Isopropanol (refilled monthly) so that the freezing process is as quick as possible.

2. Cryovials should be pre-labelled and pre-opened in the BSC.

3. Begin adding freeze media slowly dropwise to the cell pellet using a 25 ml stripette, making sure to swirl at the same time.

4. Using the same 25 ml stripette, take up as much of the cell and freeze media solution and quickly add 1 ml to the prepared cryovials.

5. Quickly cap the tubes and add them to a Mr. Frosty container, then move the Mr Frosty to a −80° C. Freezer.

6. Cells should be transferred to liquid nitrogen the following day on dry ice only.

### Controlled-Rate Freezer

1. Ensure the pressurized liquid nitrogen tank is filled 1-2 days before isolation and check the liquid nitrogen level before use.

2. If cryovials are to be used, they should be pre-labelled and pre-opened in the BSC.

3. Centrifuge cell suspension.

4. Run the controlled rate freezer “T Cell” setting, as per WI-10 Controlled-Rate Freezer with attached Low Pressure Liquid Nitrogen Supply Tank.

5. Add freeze media slowly dropwise to the cell pellet using a 25 ml stripette while swirling.

6. Using the same 25 ml stripette, take up as much of the cell and freeze media solution and quickly add 1 ml into the vials.

7. While still in the BSC, quickly cap the tubes.

8. Add samples to the controlled-rate freezer when it reaches a temperature of 4° C.

9. Cells should be transferred to liquid nitrogen immediately after the freezer has completed its run (i.e. when it has reached −180° C.).

### T Cell Initiation

### Day 0—Thaw

This protocol follows the procedure for the use of a single donor; different donors should not be mixed.

1. Prepare media and warm to 37° C.

2. Remove required number of PBMC vials from liquid nitrogen.
2.1. Due to donor to donor variability in PBMC growth, it can be necessary to initiate more PBMCs than the number of T cells needed on day 3 due to differences in the extent of PBMC expansion.

3. Prepare one 50 ml tube with 10 ml pre-warmed media in a BSC (10 ml for every vial of cells to be thawed).

4. Thaw PBMC vials in a 37° C. water bath with occasional gentle swirling until a small piece of ice remains (no more than two vials to be thawed at a time).

5. Return vials to BSC, and add 1 ml of warmed media slowly to the vial and pipette up and down once to mix.

6. Transfer contents of vial slowly to the appropriately labelled 50 ml falcon of pre-warmed media.

7. Rinse the vial with medium and transfer this to the 50 ml tube.

8. Bring the volume up to 50 ml with T cell growth medium (TCGM).

9. Centrifuge at 300×g for 7 mins at RT.

10. In BSC, aspirate supernatant to waste.

11. Re-suspend in 10 ml TCGM.

12. Count cells using Nucleocounter.
12.1. To count cells, take aliquot (50 μl) of cell suspension and dilute with 450 μl media in an Eppendorf to a final volume of 500 μl.
12.2. Use Via-1 cassette and the Nucleocounter to count cells—include dilution on software (i.e. type “50” in the sample box and “450” in the dilution box—The Nucleocounter will calculate the dilution factor for you).
12.3 Visually inspect gate on software and alter to include main body of cells if necessary, and record cell viability and concentration.

13. Bring cells up to volume with TCGM so they are at 1×10 6/ml.

14. Add 0.5 μl of both CD3 and CD28 antibodies per million cells.

15. Seed cells into appropriately labelled flasks for initiation.
15.1. Up to 30 ml/T25.
15.2. Up to 80 ml/T75.

16. Place flasks lying down (horizontally) in a 5% CO₂ buffered, water jacketed incubator at 37° C.

17. Cells should remain untouched for ˜72/96 hours (Seeded Monday for use on Thursday, or seeded Friday for use on a Monday/Tuesday).

18. Cells will appear ‘flaky’ or ‘snow-like’ after 3 days in the flask when they have initiated well.

### Example 7: Solupore™ System of PBMC Initiated T Cell Culture

The SOLUPORE™ process of PBMC initiated T cells takes place in a functionally closed instrument.

### Relevant Acronyms

BSC: Biosafety Cabinet

d: diameter

EtOH: Ethanol

GFP: Green Fluorescent Protein

PBS: Phosphate Buffered Saline

Pen/Strep: Penicillin Streptomycin

PETE: Polyester Track Etched

PC: Polycarbonate

PS: Polysulfone

SS: stainless steel

Al: Aluminium

WFL: Water for injection

mRNA: messenger ribonucleic acid

### Materials & Equipment

System Equipment

| Equipment | Asset or Product Number |
|---|---|
| Biological Safety Cabinet (BSC) | Safefast Classic 212 |
| Incubator | Galaxy CO-170S |
| Analytical Balance | TP-214, EX125D, PAS214C, |
| | ML2001, ML203E |
| Research plus 10 mL pipette | Eppendorf 3120000089 |
| 1 mL Pipette | Eppendorf Z683825 |
| 200 μL Pipette | Eppendorf Z683817 |
| 5 mL Pipette | Eppendorf Z683833-1EA |
| Timer | VWR 11852031 |
| Glass beaker for waste media | VWR 213-0016 |
| ChemoMetec Nucleocounter | 970-3002/970-0200 |
| (NC) 200 or 3000 | |
| Tweezers | Radionics 136-9745 |
| Infrared Thermometer | VWR 620-1924 |
| Scissors | N/A |
| Allen key | N/A |

### System Components.

| Component | Asset or Product Number |
|---|---|
| System Chamber Base Plate (1/4-28 | M-1221-0015-01 |
| drain thread) System Stand | |
| System Single Target Chamber Lid | M-1221-0003-01 |
| System Single Target Chamber | S-1121-002-01 |
| Lid Subassembly | |
| System Chamber Mask- | N/A |
| Polycarbonate | |
| System Chamber Mask - Stainless | M-1221-0004-01 |
| Steel | |
| Filter Membrane Holder - Polysulfone | N/A |
| Filter Membrane Holder Aluminium | M-1221-0006-01 |
| Atomiser Reservoir Module | |
| Clippard Lid Module Mount | |
| Eppendorf Clamp | M-1217-0018-03 |
| System Sample Reservoir Sip | M-1217-0020-01 |
| Tube | M-1214-0050-02 |
| Pinch Valve Assembly 300 mm 0.03″ | |
| ID 0.06″ OD | P-CP02-0004-01 |
| Silicone Tubing | |
| Microfluidic Reservoir for 1.5 | P-CP02-0006-01 |
| mL Eppendorf ® - XS | P-EV01-0001-02 |
| System Eppendorf Mount Bracket | M-1217-0019-02 |
| Knurled Thumb Knob | |
| M4 × 10 mm | P-MS01-0141-02 |
| Whatman PolyVENT 1000 Filter | P-GL01-0001-01 |
| Capsule | |
| Pressure regulator | N/A |
| Solenoid valve | N/A |
| Ballast tank 750 ml | N/A |

| Consumables | Supplier | Product Number |
|---|---|---|
| PETE 3.0 μm 110 mm d filters | Sterlitech | 2050094 |
| Drain discs 110 mm d | Sterlitech | 2050098 |
| 1.5 mL Eppendorf | Fisher | 11558232 |
| | Scientific | |
| 5 mL Filter Pipette tips | Merck | Z640271 |
| 200 μL Filter Pipette Tips | Thermo Fisher | 94052300 |
| | Scientific | |
| pH paper 148 × 105 mm | Johnson Test | N/A |
| | Papers | |
| Calibration Collection Cup | N/A | N/A |
| WIPER FOAMTEC PU Contec | VWR | 115-1858 |
| Foamtec Laundered Wipe | | |
| Luer/Spike Interconnector | Miltenyi | 130-122-744 |
| | Biotec | |
| 150 ml Teruflex Transfer bags | Terumo BCT | BB*T015CM |
| Sartorius ™ Minisart ™ NML | Fisher | 10076891 |
| Syringe Filters, Sterile 0.2 um | Scientific | |
| CleanCap EGFP mRNA (5 moU) | Tebu Bio | L-7201-1000 |
| CTS Optimiser (Basal Media) | Thermo Fisher | A1048501 |
| VIA-1 Cassettes | ChemoMetec | 941-0012 |
| 50 mL Falcon Tubes | Sarstedt | 62.559.001 |
| Universal LL Stopper | Medguard | UN940 |
| Male/Female - Red | | |
| 60 mL Luer lock Syringes | VWR | 613-1998 |
| T25 Tissue Culture Flask | Thermo Fisher | 169900 |
| | Scientific | |
| S-buffer 20X | Avectas | N/A |
| Ethanol | Sigma Aldrich | 459844-500ML-D |
| PBS (1X) | Life | 70011-069 |
| | Technologies | |
| WFI | VWR | HYCLSH30221.LS |

### System Consumables.

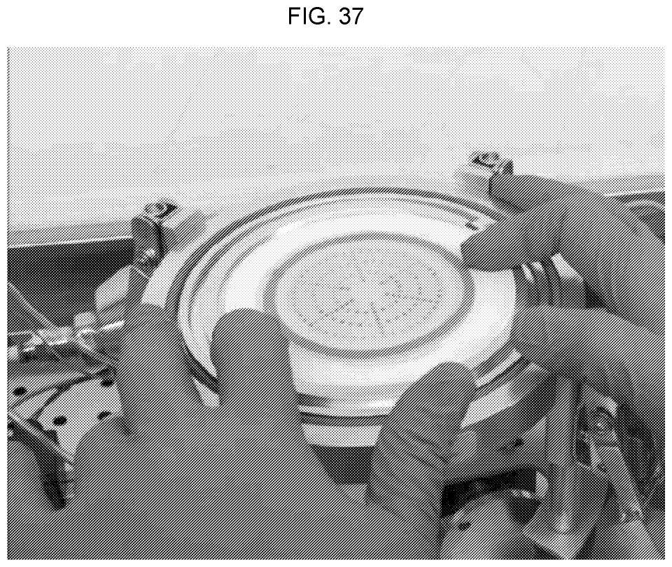

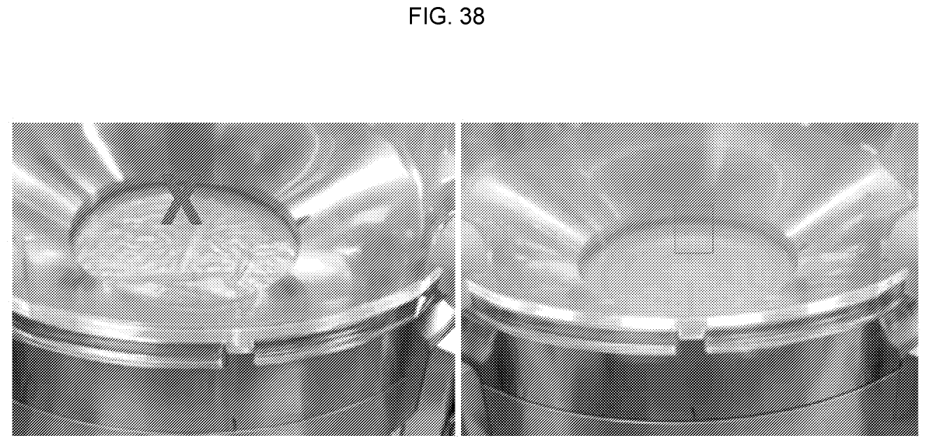

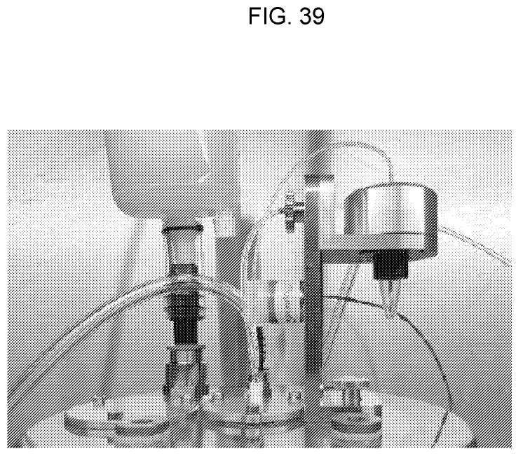

### System Consumables

| | | Avectas part | | |
|---|---|---|---|---|
| | Description | number | Supplier | Quantity |
| S-1221-0006-01 | Oring 4: BS Ref 124 | P-MC01-0017-01 | McMaster | 1 |
| System Chamber | (1283N67) | | Carr | |
| Lid O-rings | Xring 1: BS Ref 117 | P-MC01-0018-01 | McMaster | 1 |
| (FIG. 37) | (6540K141) | | Carr | |
| S-1221-0005-01 | O-ring 1 | P-MC01-0014-01 | McMaster | 1 |
| System Chamber | | | Carr | |
| Mask and Base O-rings | O-ring 2 | P-MC01-0015-01 | McMaster | 1 |
| (FIG. 38) | | | Carr | |
| | O-ring 3 | P-MC01-0016-01 | McMaster | 1 |
| | | | Carr | |
| S-1221-0007-01 | Gasket 1 (1.6 mm thick) | M-1221-0013-01 | McMaster | 1 |
| System Chamber | | | Carr/NE Seal | |
| Gaskets | Gasket 2 (2 mm thick) | M-1221-0014-01 | McMaster | 1 |
| | | | Carr/NE Seal | |
| S-1221-0003-01 | System Fluid Transfer Tube | S-1221-0004-01 | Avectas | 3 |
| System Chamber | ¼-28 UNF to FLL | P-NM01-0001-01 | Nordson | 3 |
| Fluid Inlet/Outlet | | | Medical | |
| Tube Sets | MLL to MLL union | P-QS01-0001-01 | Qosina | 1 |
| (FIG. 39) | 0.03″ × 0.065″ × 150 mm | M-1221-0016-01 | Clippard | 1 |
| | medical grade silicone tubing | | | |

### Solution Preparation

All solution preparation must be carried out in a BSC.

1. Stop Solution: Prepare 50 mL of Stop Solution by mixing 25 mL of PBS with 25 mL WFI in a labelled 50 mL falcon tube and place on ice until required. Do not use PBS opened >1 month. Record date opened in run record.

2. Cell Culture Media: Calculate media requirement based on total number of samples and resuspension volume. Prepare complete cell culture media (+0.5% Pen/Strep) or obtain an aliquot of previously prepared media (120 mL aliquots). Keep at room temperature.

3. Calibration Solution: Prepare min 10 mL volume of Payload Free Delivery Solution for Calibration of atomisers by adding to a 50 mL Falcon tube labelled “Calibration” volumes in this order first WFI, then EtOH (final 12%) and finally S-Buffer (20× prepared), detailed in (Table 5). NB Do not add S-buffer and EtOH solutions together as this will result in precipitation of excipients in S-buffer. Ensure WFI is added first.
3.1. Elveflow Reservoir: In the BSC, transfer 1 mL of “Calibration” solution to a labelled 1.5 mL sterile Eppendorf tube. If sample number (number of Sprays) for SOLUPORE™ process was known continue to 4 SOLUPORE™ process Solution Preparation. If sample number (number of Sprays) for SOLUPORE™ process is not known continue to 5 Assembly and Calibration and when sample number known prepare SOLUPORE™ process Solution.

Payload free Delivery Solution for Calibration of atomisers.

| Payload Free | WFI | EtOH | S-buffer 20X |
|---|---|---|---|
| Delivery Solution | (mL) | (mL) | (mL) |
| 10 mL | 8.3 | 1.2 | 0.5 |

4. SOLUPORE™ process Solution: Calculate the required volume of Delivery Solution for SOLUPORE™ process of 2.4×10\hat{ }7 T cells with 50 μL spray volume using calculations in Table 6, example given for 10 sprays.

Calculation for Total Volume of Delivery Solution
required for SOLUPORE ™ process

| | Eveflow/Syringe |
|---|---|
| | Reservoir System |
| Ten sprays (50 μl each) * | 50 μl × 10 = 500 μl |
| Atomiser Priming volume * | 50 μl × 1 = 50 μl |
| Excess Volume (per sample)** | 100 μl |
| Total Volume | 750 μl |

* Please note with changing atomisers Delivery Volume, Prime and Dead volumes may be altered, and the above calculation amended accordingly

**Excess volume accounts for the 10% excess that is allowed during calibration.
4.1. Refer to Table 7 to calculate the required volumes of Payload and Delivery Solution components for the required number of experimental sprays and record in run record. Example given for 10 sprays at 0.1 μg/mL and 0.4 μg/mL GFP concentrations.
4.2. Within a BSC, add the delivery solution components to a 1.5 ml Eppendorf tube labelled “SOLUPORE™ process” and store on ice until required. NB Do not add S-buffer and EtOH solutions together as this will result in precipitation of excipients in S-buffer. Ensure WFI is added first. Do not add Payload (GFP mRNA) at this stage.
   Payload and Delivery Solution composition for Syringe & Elveflow Reservoirs.

| | | % Composition in | | | % Composition in | | |
|---|---|---|---|---|---|---|---|
| | | Final Delivery | Vol required for | | Final Delivery | Vol required for | |
| | | Solution | 10 sprays @ 50 μl | | Solution | 10 spray @ 50 μl | |
| | | 0.1 · g/ | per Target | | 0.4 · g/ | per Target | |
| Order of preparation | | μl mRNA GFP | Total vol = 750 μl | | μl mRNA GFP | Total vol = 750 μl | |
| 1. | WFI | 73% | 391.5 | μl | 43% | 172.5 | μl |
| 2. | Ethanol | 12% | 90 | μl | 12% | 90 | μl |
| 3. | 20X S.Buffer | 5% | 187.5 | μl | 5% | 187.5 | μl |
| 4. | eGFP mRNA | 10% | 75 | μl | 40% | 300 | μl |
| | (1 mg/ml) | | | | | | |

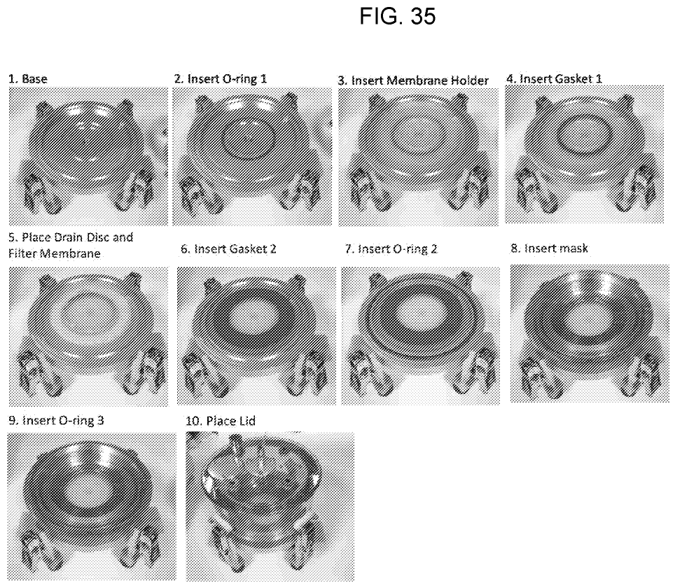

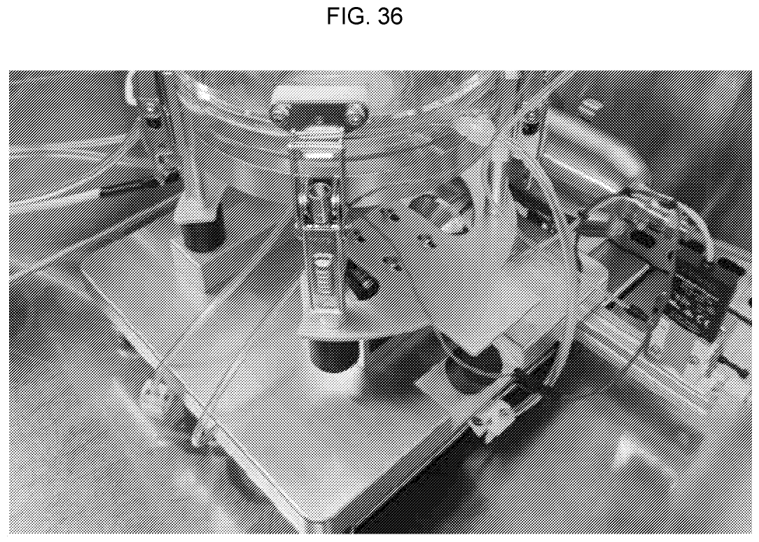

5. Assembly of system, calibration of the atomiser and priming of the filter membrane
5.1. Using the check list provided, transfer all the necessary bags containing components and consumables from the EtO cabinet into the BSC to assembly the system unit (FIG. 35 and FIG. 36).
5.2. Using the check list provided, aseptically transfer into the BSC the consumables and labware required for one experimental run.
5.3. Unpack sterilised components and dispose of the bags.
5.4. To assemble the lid (FIG. 36):
   5.4.1. Place X ring to seal the atomiser (a)
   5.4.2. Insert the atomiser equipped with airline (b)
   5.4.3. Add the cap to fix the atomiser in place (c)
   5.4.4. Use the allen key provided to tighten the screws of the atomiser's cap (d)
   5.4.5. Mount the bracket containing the Clippard valve, as shown in (e)
   5.4.6. Secure the bracket by screwing screw (f)
   5.4.7. Mount the reservoir holder and Elve flow module (g)
   5.4.8. Connect the silicon tube to the reservoir needle (h). On the unit control select settings and press Purge, the clippard valve will open then insert a segment of the silicon tube into the Clippard valve.
   5.4.9. Connect the other end of the silicon tube to the needle in the atomiser sample channel. Ensure that the silicon tube below the clippard valve is straight. If not press Purge to open the clippard valve and straighten.
   5.4.10. Connect Clippard valve wire as illustrated (j).
   5.4.11. Connect the airline of the atomiser to the solenoid valve.
   5.4.12. Connect the venting tubing and filter (k)
8.5.5. Place the system base (FIG. 35) onto the tilting stand as in FIG. 36.
5.6. Screw the three 1/4-28 luer locks provided into the drain port, bleed port and cell inlet/outlet port.
5.7. Connect the luer locks to their respective tube and ensure all clamps a closed.
5.8. Connect a 0.2 μm filter to the end of the bleed port tube and connect waste bag to the drain port tube.
5.9. Insert the o-ring 1 (thicker o-ring) into the groove around the drain area (FIG. FIGS. 35 and 36).
5.10. Place the membrane holder onto the base (FIGS. 35-37).
5.11. Place the gasket 1 onto the membrane holder (FIGS. 35-38).
5.12. If assembling for SOLUPORE™ process Filter Priming add the drain disc (FIGS. 35-39) continue to assembling for Calibration or Loading SOLUPORE™ process Solution add blue filter divider in place of drain disc and filter membrane (FIGS. 35-39). Continue to 5.15.
5.13 Moisten disc by pipetting 1 ml 1×PBS. Ensure drain port is open. Gently press down the edge of the drain disc to facilitate uniform wetting of the drain disc and rotate the disc to disperse liquid, as illustrated in FIG. 37. Do not touch the drain disc within the area delimited by gasket 1.
5.14. Place the PETE filter membrane on the drain disc.
5.15. Place gasket 2 on the filter membrane (FIGS. 35-40) or blue filter divider if preparing for calibration only. Note for SOLUPORE™ process: When placing gasket 2 pay attention to avoid crinkling of the filter. If the filter shows crinkles (FIG. 38), dispose of it and start again with a new one. Note for SOLUPORE™ process: To aid placement of the filter membrane between sample runs and empty syringe can be connected to the drain tube. Using this syringe gentle pull a vacuum to help maintain a flat filter membrane prior to placing gasket 2 on top.
5.16. Insert o-ring 2 (FIGS. 35-41).
5.17. Place the PS or SS mask (FIGS. 35-42).
5.18. Insert o-ring 3 (FIGS. 35-42).
5.19. Open clamp on drain tube.
5.20. Place on unit lid, without clamping.
5.21. For Calibration continue to 5.22 and for SOLUPORE™ process Filter Priming proceed to 8.7.

### Calibration

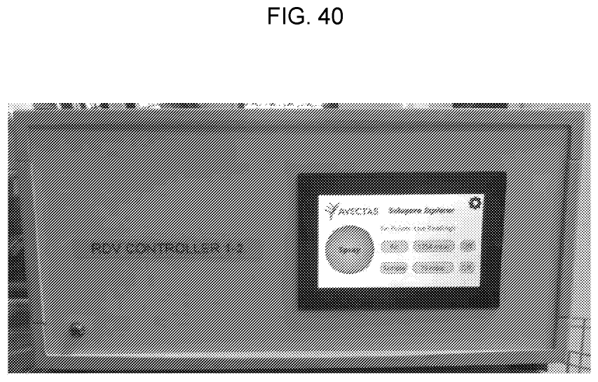

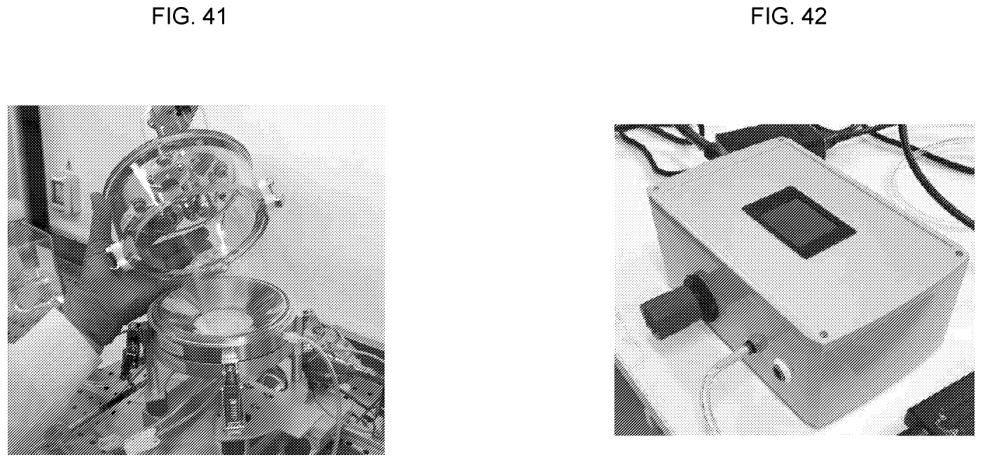

5.22. Prime the atomiser with Calibration Solution.
5.23. Unscrew the base of the Elveflow unit counterclockwise (FIG. 39), remove the 1.5 mL Eppendorf tube (to protect needle during storage). Place the “Calibration” tube into the base and screw the Elveflow unit back together.
5.24. On the controller FIG. 40, select “Air” and set pressure to 1730 mbar and then select “Sample” and set pressure to 50 mbar.
5.25. Remove the lid and place 70% IPA cleaned calibration cup underneath the atomiser.
5.26. Actuate the “Spray” button until spray appears at atomiser tip (normally 2-3 times).
5.27. Set the Sample pressure to 100 mbar or start with the calibrated setting from previous days experimental run.
5.28. Actuate the “Spray” button twice, confirm spray occurred by visual inspection and remove waste container.
5.29. To Calibrate the atomiser: 5.30. Using a scissors cut the top rim of 70% IPA cleaned calibration cup.
5.31. Tare the cup on the analytical balance.
5.32. Place the cup in the MID 1T chamber, as illustrated in FIG. 41.
5.33. Place the lid on the unit and simultaneously with uniform pressure close 2 opposite clamps.
5.34. Confirm clamp on venting port is open.
5.35. Close clamps on drain tube, cell inlet/outlet tube and bleed port tube.
5.36. Calibration of the atomiser:
   5.36.1. Confirm the Air pressure is set to 1730 mbar.
   5.36.2. Set Sample pressure to last calibrated value record on run record.
   5.36.3. Set Airflow through showerhead at the required pressure e.g. 90 mbar using the manual pressure regulator (FIG. 42).
   5.36.4. Open clamp on Airflow line to showerhead.
   5.36.5. Actuate the ‘Spray’ button.
   5.36.6. Close clamp on Airflow line to showerhead.
   5.36.7. Immediately unclamp the lid, remove calibration cup and replace lid on unit.
   5.36.8. Weigh calibration collection cup immediately.
   5.36.9. Record weight in Run Record.
   5.36.10. Spray the cup liberally with 70% IPA and wipe dry.
   5.36.11. If weight >55 mg* (55 μL) reduce the sample pressure 10 mbar. Dispense xl spray in waste cup and repeat spray and measure.
   5.36.12. If weight <50 mg (50 μL) increase the liquid pressure 10 mbar. Dispense xl spray in waste cup and repeat spray and measure.
   5.36.13. *assumption: density of delivery solution˜1 g/ml.
5.37. Repeat calibration measurement until 3 consecutive measurements between 50-55 mg are obtained. Ensure that the calibration solution does not run dry and the whole length of the sample line is always filled with liquid.
5.38. Record optimal pressure and calibration weights in run record.
5.39. When calibration is complete, place waste cup under the atomiser. Select system setting, increase pressure settings to 500 mbar and Select press Purge Sample to expel the calibration solution from the atomiser. Repeat Purge one additional time and dispose of the waste cup.

### SOLUPORE™ Process

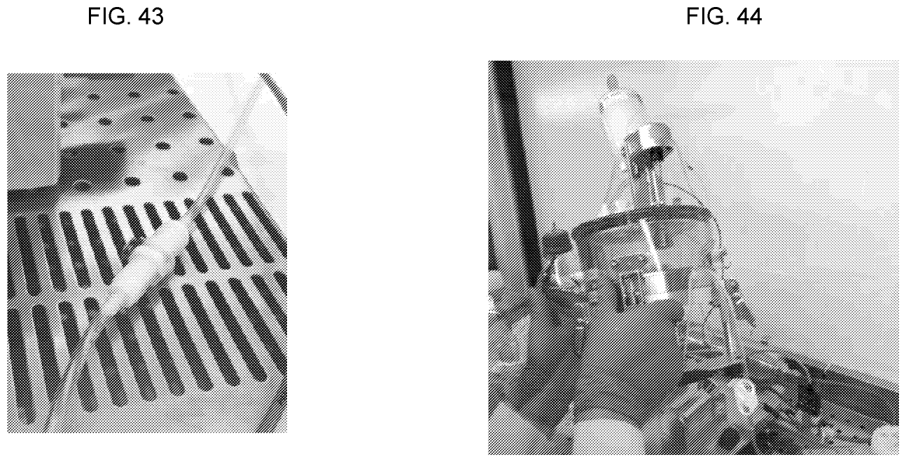

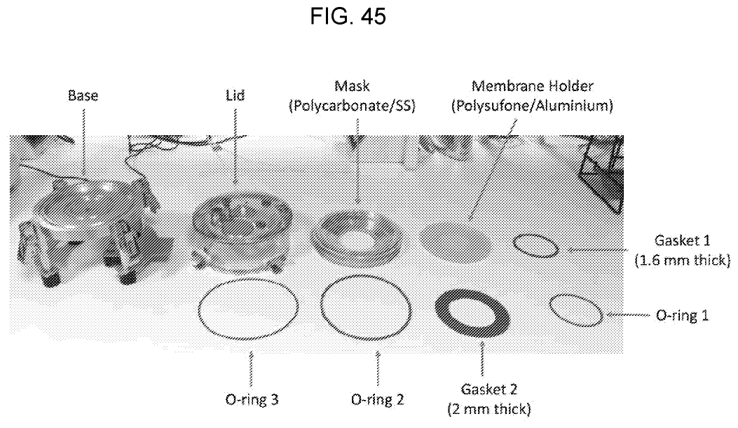

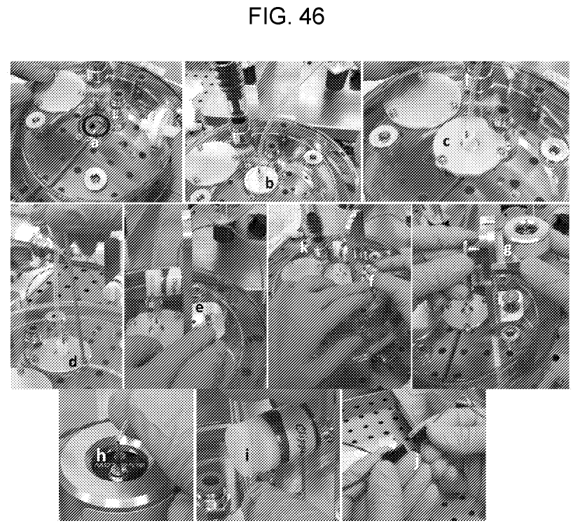

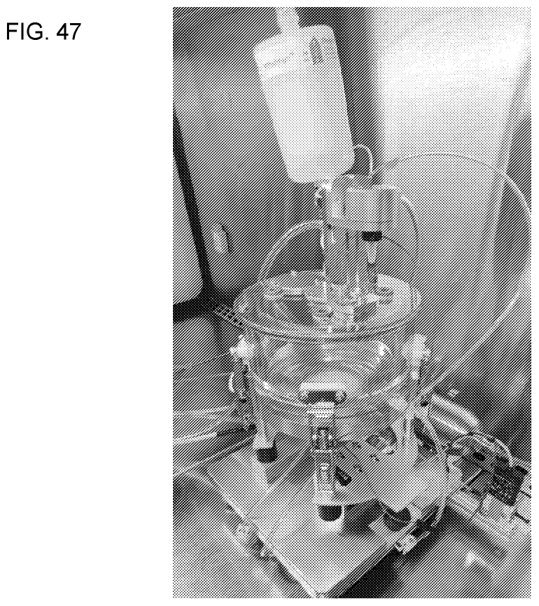

6. Load payload solution ‘SOLUPORE’ process Solution’
6.1. Disconnect the sample line from the control unit line. FIG. 43.
6.2. Load required volume of GFP into SOLUPORE™ process Solution.
6.3. Unscrew the base of the Elveflow unit counterclockwise (FIG. 39), remove the 1.5 mL Eppendorf tube (Calibration). Place the “SOLUPORE™ process” tube into the base and screw the Elveflow unit back together.
6.4. Re-connect the sample line to control unit line.
6.5. On the controller FIG. 40, select “Air” ensure pressure is set to 1730 mbar and then select “Sample” and set pressure to 50 mbar.
6.6. Remove the lid and place 70% IPA cleaned calibration cup underneath the atomiser on top of blue filter membrane divider.
6.7. Actuate the “Spray” button until spray appears at atomiser tip (normally 2-3 times).
6.8. Set the sample pressure to the calibrated value and actuate ‘Spray’ button once. Confirm spray has occurred by visual inspection.
6.9. Remove blue disc divider and assemble filter drain disc and filter membrane as described in 5.13-5.20 and continue to 7 for SOLUPORE™ process Filter Priming.

7. SOLUPORE™ process Filter Priming.
7.1. Place lid on unit, lock opposite clamps together to seal the System chamber
7.2. Ensure you have:
   4×60 ml syringes
   4× syringe caps
   1×150 ml sterile bag
   1× luer spike interconnector
   1×0.2 μm Filter on the Bleed Port tube before proceeding to priming of the filter membrane.
7.3. Take a 60 mL syringe, label “PRIME” remove the plunger, insert the syringe cap and fill the syringe with 60 mL 1×PBS. Insert plunger, remove the cap and connect the syringe to the cell inlet port.
7.4. Ensure clamps on venting and drain tubes are closed.
7.5. Open the clamp on cell inlet tube and load into 60 mL PBS into chamber.
7.6. Open the pinch clamp on the airflow tube.
7.7. Open the drain port clamp and allow about 30 mL˜50% of the PBS to drain.
7.8. Close the pinch clamp on the airflow tube and immediately open the clamp on the venting tube.
7.9. Verify that gravity draining occurs. If gravity draining is inhibited, repeat from step 8.7.3.
7.10. If required 1 mL priming solution and set aside at room temp. for sterility test.

8. Cell Loading
8.1. Record on run record
   Time cell samples were aliquoted by cell culture
   Volume of sample
   Cell concentration e.g. 24×10\hat{ }6 cells/30 mL
   Time at which cells are loaded in the chamber
8.2. Ensure cell suspension provided in basal media is at the required cell concentration.
8.3. Mix homogenously, using a 5 mL pipette set to 4 mL, gently disrupt any cell clumps by pipetting up and down 10 times.
8.4. Using a 200 μL pipette take 2×200 μL of the cell suspension and transfer each aliquot into add a 1.5 mL Eppendorf tube labelled “BS (before SOLUPORE™ process)+Sample #” for counting and flow cytometry analysis. Repeat for all samples.
8.5. Flow cytometry leave all samples in the 1.5 mL tubes in ice and perform the analysis at the end of the SOLUPORE™ process experiment.
8.6. Count in accordance with WI-06. Briefly,
   Gently mix sample by pipetting 5 times using a P200 pipette. Do not get bubbles in the sample.
   Count using Via 1 Cassette on the NC3000/NC200 (in accordance with WI-06).
   Record time of counts in run record. Ensure the same nucleocounter is used for counting all samples before and after SOLUPORE™ process.
8.7. Obtain temperature reading of Stop Solution before using for the first time and record in run record.
8.8. Disconnect and re-cap the syringe used for priming. Set aside for next run.
8.9. Take a sterile 60 mL syringe, remove the plunger, connect the syringe to a cap and gently pour the cell suspension into the syringe.
8.10. Insert the plunger and turn the syringe upside-down, remove the cap and gently expel air.
8.11. Connect the syringe to the cell inlet/outlet tube.
8.12. Ensure clamp on drain tube closed.
8.13. Open the clamp on cells inlet/outlet tube and gently press the plunger to load the cell suspension into the chamber. Keep syringe plunger down to fill inlet tubing once filled rotate syringe 1800 and add in the remaining cell suspension. Once cells are loaded remove syringe and purge the inlet line with air from air filled syringe to push any remaining liquid in the line into the chamber. Avoid introducing bubbles by tilting the chamber gentle to the left.
8.14. Close the clamp on cells inlet/outlet tube and dispose of the syringe.
8.15. Prepare a labelled syringe with 5 mL Stop Solution and another labelled syringe with 20 mL cell culture media, cap syringes and set aside.
8.16. Open the drain clamp and start timer and measure filtration time.
8.17. Filtration is considered complete when the filter looks dry by eye and there is no more liquid flow-through tube.
8.18. Record time of filtration in run record.
8.19. When no more liquid runs through the drain tubing, open the bleed port clamp, hold bleed port tube in an upright position above unit base and observe residual liquid flow-through the drain tube. Ensure all liquid removed from drain tube.
8.20. Close all clamps on drain line, on the waste bag and on bleed port tube.
8.21. Disconnect the waste bag and set aside. Connect the syringe with recovery solution—cell culture media.
8.22. Connect the syringe with Stop Solution to the inlet tube.
8.23. Turn on airflow through the showerhead (set e.g. to 90 mbar), open the clamp on the airflow line, immediately actuate the ‘Spray’ button and start 30 s timer 8.8.24. Close clamp on airflow line.
8.25. After 30 s incubation, open clamp on inlet line and add Stop Solution, incubate for a further 30 s.
8.26. Open drain clamp and rapidly flush 20 mL media into the chamber.
8.27. Tilt the chamber and gently aspirate cell suspension in and out of the chamber to the syringe 14 times. Avoid formation of bubbles by slowly aspirating the solution in and out of the syringe by gently pulling the plunger up and down.
   Ensure that entire filter is flushed by recovery solution.
8.28. Tilt chamber back to neutral position.
8.29. Harvest the cell suspension by slowly tilting the chamber to the maximum angle while slowly aspirating the cell suspension into the syringe. If bubbles observed on mask return to neutral position and tilt chamber again to aspirate bubbles into syringe.
8.30. Close the inlet clamp and disconnect the syringe.
8.31. Transfer the cell suspension into a T-25 flask.
8.32. Connect the syringe to the inlet port again to aspirate any residual cell suspension and transfer into the T-25 flask.

91 Post Recovery Analysis:
9.1. Using a 200 μL pipette take 2×200 μL of the cell suspension and transfer each aliquot to a 1.5 mL Eppendorf tube labelled “AS (after SOLUPORE™ process)+Sample # “and set aside at room temperature for counting and flow cytometry.
9.2. Weigh cell culture dish containing the Soluporated cell suspension and record weight/volume on run record.
9.3 Ensure cell culture dish is labelled: Sample name; Volume recorded; Operator name; Date
9.4. Place cell culture dish in incubator 37±2° C., 5% CO₂, 95% relative humidity in an upright position.
9.5. Count “Post-Sol” sample set aside for counting and count in accordance with WI-06) and record time of counts in run record.
   Ensure the same nucleocounter is use or counting the “Pre-Sol” and “Post-Sol” samples.

10. Inter-Sample Cleaning
10.1. Dispose of the waste bag between experimental replicates.
10.2. Unclamp and remove the lid, disassemble the filtration unit to remove and dispose of the drain disc and filter membrane.
10.3. Assemble the instrument again without inserting filters.
10.4. Clamp the chamber lid.
10.5. Ensure drain and bleed port clamps are closed.
10.6. Fill a sterile labelled syringe with 60 mL WFI, connect it to the inlet tubing and load the water into the chamber.
10.7. Hold the instrument with both ends and rotate the chamber to promote rinsing of all surfaces (FIG. 44). Tilt unit to maximum degree to ensure mask and lid wall are washed.
10.8. Aspirate with a syringe connected to the drain tubing to remove the liquid.
10.9. Repeat 10.6-10.8 using 60 mL 1×PBS.
10.10. Chamber is now ready to be re-assembled with a new drain disc and filter membrane for a new sample.

11. Repeat from paragraph. 5.13 for all other samples after unit assembly with new drain disc and filter membrane.

12. Record sample names and conditions tested in run record.

13. When the experiment has been completed, proceed to Cleaning System.

14. Export all required NC3000/NC200 files and save into appropriate experiment folder for analysis.

15. Cells are left in culture for 24 hrs and analysed for:
Cell Numbers Recovered (NC3000/NC200)
Cell Viability (NC3000/NC200)
Transfection Efficiency (% GF−Flow Cytometry)

### Example 8: Cell Thaw Culture and Preparation of Cells

The method described herein provides a standardized protocol for cell culture medium preparation, thawing cell stocks, culturing cells and preparing cells for experimental use. The method covers the preparation of cell culture medium and procedure for thawing cell stocks in liquid nitrogen storage as well as culturing cells in preparation for experimental use.

### Relevant Acronyms

### DMSO—Dimethyl Sulfoxide

### FBS-HI—Fetal Bovine Serum Heat Inactivated

### SDS—Safety Data Sheet

### PBS—Phosphate Buffered Saline

| Item Name | Supplier | Product Number |
|---|---|---|
| CTS OpTmizer Media + | Thermo Fisher | A1048501 |
| Supplement | Scientific | |
| Human AB Serum | Valley | HP1022 |
| | Biomedical | |
| Physiologix XF SR HUMAN | Nucleus | 20180806-3 |
| GROWTH FACTOR | Biologies | |
| CONCENTRATE | | |
| HEPES Buffer | Thermo Fisher | 15630056 |
| | Scientific | |
| L-glutamine 200 mM | Thermo Fisher | 25030-024 |
| | Scientific | |
| IL-2 (200 U/ml) | Cellgenix | 001420-050 |
| Penicillin/Streptomycin (10,000 | Gibco | 15070-063 |
| U/ml) | | |
| CD3 pure - functional grade, | Miltenyi | 130-093-387 |
| human (clone: OKT3) | Biotec | |
| CD28 pure - functional grade, | Miltenyi | 130-093-375 |
| human (clone: 15E8) | Biotec | |
| CTS Dynabeads | Thermo Fisher | 402030 |
| | Scientific | |
| CD3-APC antibody, human | Thermo Fisher | 17-0037-42 |
| | Scientific | |
| CD25-PE antibody, human | Miltenyi | 130-113-286 |
| | Biotec | |
| Via 1 cassette | Chemometec | 941-0012 |
| Nucleocounter slide A8 | Chemometec | 942-0003 |
| Solution 13 | Chemometec | 910-3013 |
| PBS (1X) | Gibco Life | 70011-069 |
| | Technologies | |
| Molecular Grade Water | Sigma | W4502-IL |
| 25 ml Stripette | SARSTEDT | 6.1685.001 |
| 10 ml stripette | SARSTEDT | 86.1254.001 |
| T-25 TC Flasks | SARSTEDT | 83.3910.002 |
| T-75 TC Flask | SARSTEDT | 83.3911.002 |
| 30 ml containers | Fisher | 11339633 |
| | Scientific | |
| 50 ml Falcon | SARSTEDT | 62.559.001 |
| 120 ml Falcon | SARSTEDT | 75.9922.420 |
| 500 ml Falcon | SARSTEDT | 75.9922.818 |
| 0.50 ml Eppendorf | VWR | MLBP3430 |
| 1.5 ml Eppendorf | Fisher | 11558232 |
| | Scientific | |
| Merck ™ Stericup Quick Release- | Fisher | 15959180 |
| GP Sterile Vacuum Filtration | Scientific | |
| System 0.22 μm | | |

### Equipment

### Procedure

Preparation of CD3+ T cell culture medium

| | CD3+ T cell culture media preparation | | | | | |
|---|---|---|---|---|---|---|
| | 50 ml | | 120 ml | | 500 ml | |
| CTS ™ OpTmizer ™ T Cell | 49.7 | ml | 118.7 | ml | 494.5 | ml |
| Expansion SFM containing | | | | | | |
| supplement | | | | | | |
| L-glutamine (200 mM) (0.1%) | 500 | μl | 1.2 | ml | 5 | ml |
| Recombinant human IL-2 | 50 | μl | 120 | μl | 500 | μl |
| (200 U/ml) (0.1%) | | | | | | |

1. To prepare complete medium for CD3+ T cells add a full bottle of CTS Optimizer supplement to a full bottle of CTS Optimizer media and record lot numbers for each in the cell culture template.

2. Add required volume of L-glutamine as in the above table.

3. Filter sterilise media using the vacuum Filtration System (Stericup image below) before the addition of 0.1% IL-2 (200 U/ml).

4. P/S is optional for post transfection media and will be specified by the user.

5. To use filtration system, spray vacuum and stericup with 70% ethanol before entering BSC. The vacuum pump has 2 outlets, ensure tubing is connected to the air in outlet.

6. Plug vacuum into a power supply and ensure power supply is on in BSC.

7. Remove stericup from sterile packaging and attach the nozzle of the vacuum pump to the stericup.

8. Remove clear lid from stericup and add pre-prepared medium into the top of the stericup and return lid to stericup.

9. Turn on the vacuum by using the switch at the front of the pump.

10. Medium will then go through the filter and once it has passed through the stericup, turn the pump off and remove the nozzle from the stericup.

11. Remove top of the stericup through a twisting motion and twist the sterile lid (blue) onto the bottom of the stericup containing the filter sterilised medium. There is a click when fully closed.

12. For large batch preparation of medium, store medium in clearly labelled stericups at 4° C. before aliquoting medium in 50/120 ml falcons, when necessary.

Note, medium cannot be stored for more than 7 days at 4° C.

13. Add 0.1% IL-2 (200 U/ml) for culture of T cells.

14. To prepare IL-2, Add 2 ml of sterile water to one lyophilised vial of 50 μg recombinant human IL-2, mix, and divide into 500 μl aliquots. Store at −20° C.

15. Keep all cytokines and antibiotics (optional for post transfection) on ice while thawing or before use.

16. After use, return reagents to storage locations, for example −20° C. or 4° C.

17. Warm medium using a water bath set to 37° C. for at least 15 minutes before use.

18. Record temperature of the water bath in the cell culture template.

Preparation of PBMC initiated CD3+ T cell culture medium

| | PBMC Initiated CD3+ T Cell TCGM | | | | | |
|---|---|---|---|---|---|---|
| | Complete Media Preparation | | | | | |
| | 50 ml | | 120 ml | | 500 ml | |
| CTS ™ OpTmizer ™ T Cell | 47 | ml | 112.8 | ml | 469.5 | ml |
| Expansion SFM containing | | | | | | |
| supplement | | | | | | |
| L-glutamine (200 mM) 1% | 500 | μl | 1.2 | ml | 5 | ml |
| Recombinant human IL-2 | 50 | μl | 120 | μl | 500 | μl |
| (200 U/ml) 0.1% | | | | | | |
| Human Ab Serum/Physiologix | 2.5 | ml | 6 | ml | 25 | ml |
| XF SR HUMAN GROWTH | | | | | | |
| FACTOR CONCENTRATE 5% | | | | | | |

1 To prepare TCGM complete medium for PBMC initiated CD3+ T cells add a full bottle of CTS Optimizer supplement to a full bottle of CTS Optimizer media and record lot numbers for each in the cell culture template.

2. Add required volume of Human Ab serum or Physiologix serum as in the above table.

3 Add required volume of L-glutamine as 8.3. in the above table.

4. Add required volume of HEPES buffer as in the above table.

5. Filter sterilise media using the vacuum Filtration System before the addition of P/S and IL-2.

6. To use filtration system, spray filtration system and stericup with 70% ethanol before entering BSC. The vacuum pump has 2 outlets, ensure tubing is connected to the air in outlet.

7. Plug filtration system into a power supply and ensure power supply is on in BSC.

8. Remove stericup from sterile packaging and attach the nozzle of the filtration system to the stericup.

9 Remove clear lid from stericup and add pre-prepared medium into the top of the stericup and return lid to stericup.

10. Turn on the filtration system by using the switch at the front of the filtration system.

11 Medium will then go through the filter and once it has passed through the stericup, turn the vacuum off and remove the nozzle from the stericup.

12 Remove top of the stericup through a twisting motion and twist the sterile lid (blue) onto the bottom of the stericup containing the filter sterilised medium. There is a click when fully closed.

13 For large batch preparation of medium, store medium in clearly labelled stericups at 4° C. before preparing aliquoting in 50/120 ml falcons, when necessary, and before the addition of P/S and IL-2. Note, medium cannot be stored for more than 7 days at 4° C.

14 Add IL-2 (200 U/ml) for culture of T cells. To prepare IL-2, Add 2 ml of sterile water to one lyophilised vial of 50 μg recombinant human IL-2, mix, and divide into 500 μl aliquots. Store at −20° C.

15. Optional post transfection: Add P/S (10,000 U/ml) (250 μl per 50 ml medium)

16. Before use, warm medium using a water bath set to 37° C. for at least 15 minutes.

### Thawing and Activation of Cells

1. Prepare CD3+ T cell media or complete TCGM media and warm to 37° C.

2 Prepare CTS™ OpTmizer™ T Cell Expansion SFM containing supplement alone (this is basal medium) and warm to 37° C.

3. Record the temperature of the water bath in the cell culture template.

4. Prepare one 50 ml tube with 10 ml pre-warmed media in a BSC (10 ml for every vial of cells to be thawed).

5 Retrieve required vials/bags from liquid nitrogen/−150° C. freezer. (Note all appropriate PPE must be worn while dealing with liquid nitrogen or −150° C. freezer)
5.1 Due to donor to donor variability in PBMC growth, it can be necessary to initiate more PBMCs than the number of T cells needed on day 3 due to differences in the extent of PBMC expansion. This number should be assessed the first few times thawing a new donor.

| Cell type | Cell number/vial | Cell request | Number of vials |
|---|---|---|---|
| CD3+ T cells | 20 × 10⁶ | 40 × 10⁶ | 4 |
| PBMC | 50 × 10⁶ | 60 × 10⁶ | 2 |

6. Thaw cell vials in a 37° C. water bath with occasional gentle swirling until a small piece of ice remains (no more than two vials to be thawed at a time). Ensure the rim of the vial does not come in contact with water from water bath as this is a contamination risk. 7 When the ‘ice pebble’ is visible, move to the BSC, spray and wipe vials down with 70% EtOH, and dilute the cells rapidly in culture medium by adding 1 ml of warmed media to the cryovial using a P1000 pipette or 3 ml to the cryobag using a luer lock syringe.

8. Add the contents of the vial/bag to the pre-prepared media in a falcon. Note: Thawed cells should not be left for any extended period of time in the freezing mixture as DMSO is toxic to cells.

9. Rinse the vial with another 1 ml of medium to ensure all cells have been retrieved and transfer to the same falcon containing the cells.

10. Centrifuge falcon at 300×8.10. g for 7 mins at RT.

11. Check for a cell pellet and remove supernatant and discard to a waste beaker without disturbing the pellet. This can be done by pouring or removing using a strip or Pasteur pipette.

12. Resuspend pellet in culture media to a cell density greater than 1×10 6/ml (usually 10 ml for a CD3+ T Cell vial, 20 ml for a PBMC vial or 30 ml for bag).

13. Count cells using a Via-1 cassette. (see WI-6 NC-3000 NucleoCounter Operation & Maintenance or summary below).
13.1. To count cells, take 50 μl of cell suspension and dilute with 150 μl media in an Eppendorf to a final volume of 200 μl.
13.2. Use Via-1 cassette and the Nucleocounter to count cells—include dilution on software (i.e. type “50” in the sample box and “150” in the dilution box—The Nucleocounter will calculate the dilution factor).
13.3. Visually inspect gate on software and alter to include main body of cells if necessary, and record cell viability and concentration (cells/ml) and size in cell culture template.

14. Calculate the total cell count and record all calculations and cell numbers in the cell culture template.

15. Cells are seeded at a density of 1×10 6/ml in appropriately labelled cell culture flasks or bags.

| Number of cells | Flask | PBMC | CD3 T cells |
|---|---|---|---|
| <40 × 10⁶ | T25 | Lying down in incubator | Upright in incubator |
| 40-80 × 10⁶ | T75 | Lying down in incubator | Upright in incubator |
| >100 × 10⁶ | T175 | Lying down in incubator | Upright in incubator |

16. For CD3+ T cells place TC flasks upright in the incubator.
16.1. For CD3+ T cells allow cells to rest after the thawing process in a 37° C. incubator for at least 4 hours before proceeding to the activation step.
16.2. To activate CD3+ T cells, add a ratio of 2:1 CTS Grade Dynabeads: cell (5 μl per 1 million cells).
   16.2.1. For example, 100×106 cells, add 500 μl of CTS Dynabeads to cells and swirl the flask gently to ensure Dynabeads are evenly distributed amongst the whole cell population in the TC flask.
16.3. Place cells back in the incubator, upright, until the cells are required for experimental use.
16.4. Prepare cells for experimental use 19 hours post-activation unless otherwise stated.

17. PBMC's are activated immediately post thaw with soluble CD3 pure functional grade, human (clone: OKT3), and CD28 pure functional grade, human (clone: 15E8) antibodies.

18. Ensure CD3 and CD28 antibodies remain at 4° C. and store on ice until the time of activation.

19. Ensure CD3 and CD28 antibodies are within one month's date from when first opened, colour labelled, and contain users' initials. Upon opening the date and initials of user must be written on vial. All users must have their own set of CD3 and CD28 antibodies. No sharing among users is permitted.

20. Record CD3 and CD28 lot number and expiry date in the cell culture template.
20.1. For 1×PBMC activation add 0.5 μl each of CD3 and CD28 antibodies per million cells.
   20.1.1 For example, for 1× activation of 100×10 6 PBMC, add 50 μl of CD3 antibody and 50 μl of CD28 antibody to falcon of cells and invert the 50 ml tube gently to ensure antibodies are evenly distributed amongst the whole cell population.
20.2. For 2×PBMC activation add 1 μl each of CD3 and CD28 antibodies per million cells.
20.3. Seed cells into appropriately labelled T75 flasks for initiation.
20.4. Gently place flasks lying down (horizontally) in the incubator.
20.5. Cells should remain undisturbed for ˜72-96 hours.
20.6. Cells will appear ‘flaky’ or ‘snow-like’ 72 hours post initiation.
20.7. Always examine cells under the microscope for the appearance of healthy clumps of PBMCs.

### Preparing CD3+ T Cells for Experimental Use

1. At 19 hours post activation, remove the flask containing the CD3+ T cells from the incubator to the BSC.

2. Gently agitate the cell-Dynabead suspension with a stripette and transfer to a 50 ml falcon tube.

3. Wash the flask with 5 ml media and transfer to the same falcon tube.

4. Vortex the falcon tube well and place in the 50 ml magnet.

5. Wait 2 minutes.

6. Using a 25 ml stripette and without touching the edge of the falcon carefully pipette down to the 5 ml mark collecting most of the media—this media contains de-beaded cells and is collected in a fresh labelled sterilin.

7. Approximately 5 ml of media is left at the bottom of the falcon to avoid disturbing the beads.

8. Remove the falcon tube from the magnet, add a further 5 ml of media and mix again.

8.9.

9. Repeat the incubation of the falcon tube in the 50 ml magnet and collection of media. (steps 5-9).

10. Remove the 50 ml falcon from the magnet for the second time.

11. Using a P1000 pipette collect the dynabeads using the media leftover at the bottom of the falcon tube.

12. Put this media containing the beads into a 1.5 ml Eppendorf and place in the Eppendorf (small) magnet for 2 minutes.

13. Remove the media and pipette into the sterilin.

14. Add 1 ml of media to Eppendorf and mix to wash. Add to Eppendorf magnet and remove supernatant to sterilin.

15. Discard Eppendorf containing beads.

16. Centrifuge sterilin containing cells at 300×g for 7 minutes.

17. Resuspend cells in 10 ml of fresh media if expected cell count/ml is <50×10\hat{ }6 cells. When resuspending always use a P1000 first to break up the cell pellet.

18. Resuspend cells in 20 ml of fresh media if expected cell count/8.18. ml>100×10\hat{ }6 cells.

19. Prepare a dilution of cells by adding 50 μl of the cell suspension to 150 μl of media in an eppendorf—count this using a Via-1 cassette. All counts should be done in duplicate for increased accuracy.

20. Record cell count/ml, cell viability and cell size post de-bead in the cell culture template.

21. Calculate the total cell count/ml and the volume of cell suspension required for the experiment.
21.1. For Example, upon cell count there will be 4×10⁶ cells/ml in 20 ml, the total cell count is 80×10⁶. 2 parameters at 20×10⁶ in 10 ml aliquots are desired.
21.2. Want/Have=20×10⁶/4×10⁶=5×2.5 parameters=12.5 ml of cell suspension to 12.5 ml of CTS OpTmizer Media+Supplement. This is the master mix, always make up 0.5 of a condition extra. For example, if there are 2 parameters always make up 2.5 times, thus final volume of 25 ml when one needs 20 ml. This allows for pipetting error.

22. Prepare a cell master mix containing cell suspension (added using a P5000) and CTS OpTmizer Media+Supplement, in either a 120 ml falcon or a 500 ml falcon (depending on desired volume), mix gently by inversion and aliquot 10 ml cell suspension white cap 30 ml tubes using a P5000 or P10000 pipette for accuracy. Close master mix and invert a few times after aliquoting every 3 tubes to ensure a homogenous cell suspension is maintained.

23. Ensure 30 ml white cap sterlins are clearly labelled with experiment number, donor number, cell number and initials of whoever prepared the sample.

24. For untreated cell samples, ensure cells are prepared in a T25 TC flask containing CD3+ T cell complete medium, not CTS OpTmizer Media+Supplement. UT cells are exponentially growing to ensure good viability on day 1 after experiment seed at 0.5×10⁶/ml ie 10×10⁶ cells in 20 ml.

### Preparing PBMC Initiated CD3+ T Cells for Experimental Use

1. At 72 hours (day 3) post initiation remove the T75 flask of the PBMC initiated CD3+ T cells from the incubator to the BSC. Observe visually for clumping, this is a sign of cells that have initiated properly.

2. Transfer cell suspension to a 50 ml centrifuge falcon.

3. Centrifuge 50 ml falcon containing cells at 300×g for 7 minutes.

4. Resuspend cells in 20-50 ml of fresh pre-warmed media, using a P1000 to break up the cell pellet first. Use 20 ml if less than 50×10⁶ cells is expected and 50 ml if over 100×106.

5. Prepare a dilution of cells by adding 50 μl of the cell suspension to 150 μl of media in an eppendorf—count cells using a Via-1 cassette (Refer to WI-6 NC-3000 NucleoCounter Operation & Maintenance).

6. To ensure PBMCs have activated and initiated, stain cells with CD3 (T cell purity) and CD25 (T cell activation) antibodies for flow cytometry analysis. Cells cannot be released for experimental use before this staining is complete analysed on the flow cytometer.
6.1. To stain for CD3 and CD25 expression, calculate the volume of cell suspension required for 1×10⁶ cells.
6.2. Remove the volume of cells that give 1×10⁶ cells, transfer to a 1.5 ml eppendorf and centrifuge for 5 minutes at 300×g.
6.3. After centrifugation, remove cell supernatant and resuspend cell pellet in 100 μl of FACs buffer.
6.4. Compensation beads are used when staining for CD25 and CD3 antibodies to optimize the fluorescence compensation settings for flow cytometry analysis.
   6.4.1 Prepare compensation beads for both the CD25 and CD3 antibodies as follows:
      6.4.1.1. Label two 1.5 ml eppendorf tubes CD25 comp and CD3 comp.
      6.1.1.2. Mix the compensation beads by vortexing. Add one drop of compensation beads to each tube and then add 0.5 μl of the CD25 or CD3 antibody to the appropriate tube.
6.5. Once the compensation beads are prepared, add 5 μl of both CD25 and CD3 antibodies to the eppendorf containing 1×10⁶ cells.
6.6. Incubate the cells with the antibodies for 10 minutes at 4 degrees.
6.7. After 10 minutes, wash the cells by adding 1 ml of FACs buffer and centrifuge for 5 minutes at 300×g.
6.8. After centrifugation, discard the cell supernatant and resuspend the cell pellet in 100 μl of FACs buffer.
6.9. Flow cytometry is used to analyse CD25 and CD3 expression by acquiring 10,000 events for both bead and cell samples.
   6.9.1. Acquire the compensation bead samples before the cell samples.

7. If cell sample yields >90% for both CD25+ and CD3+ staining on the flow cytometer, cells are released for experimental use.

8. If the cell sample yields <90% both CD25+ and CD3+ staining on the flow cytometer, inform the end user and do not release cells for experimental use unless instructed otherwise.

9. Once confirmed for release calculate the total cell count/ml and the volume of cell suspension required for the experiment based on the nucleocounter counts. All counts should be done in duplicate for increased accuracy. (These calculations should be done during the flow incubations but don't prepare the cells until the cells have passed the QC for experimental release).
9.1. For Example, upon cell count there will be 4×10⁶ cells/ml in 20 ml, the total cell count is 80×10⁶. 2 parameters at 20×10⁶ in 10 ml aliquots are desired.
9.2. Want/Have=20×10⁶/4×10⁶=5×2.5 parameters=12.5 ml of cell suspension to 12.5 ml of CTS OpTmizer Media+Supplement. This is the master mix, always make up 0.5 of a condition extra. For example, if there are 2 parameters always make up 2.5 times, thus final volume of 25 ml when one needs 20 ml. This allows for pipetting error.

10. Prepare a cell master mix containing cell suspension (added using a P5000) and CTS OpTmizer Media+Supplement, in either a 120 ml falcon or a 500 ml falcon (depending on desired volume), mix gently by inversion and aliquot 10 ml cell suspension white cap 30 ml tubes using a P5000 or P10000 pipette for accuracy. Close master mix and invert a few times after aliquoting every 3 tubes to ensure a homogenous cell suspension is maintained.

11. Ensure 30 ml white cap sterlins are clearly labelled with experiment number, donor number, cell number and initials of whoever prepared the sample.

12. For untreated cell samples, ensure cells are prepared in a T25 TC flask containing TCGM complete medium, not CTS OpTmizer Media+Supplement. UT cells are exponentially growing to ensure good viability on day 1 after experiment seed at 0.5×10⁶/ml ie 10×10⁶ cells in 20 ml.

### Example 9: LV-eGFP (Enhanced GFP) Vector

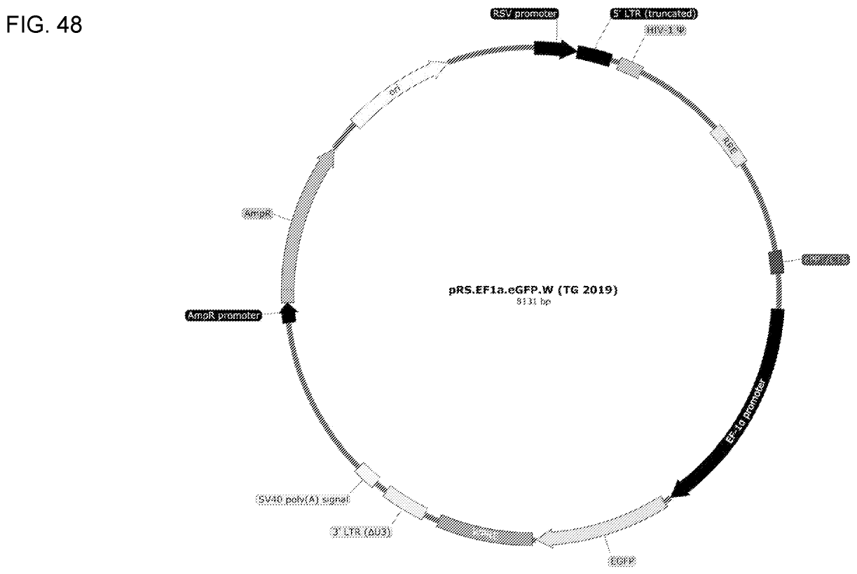

A map of the LV expression plasmid with eGFP vector is provided herein at FIG. 48.

### Example 10: Integration of GFP Post Viral Infection

ddPCR was used to look at the number of integrated copies of GFP per cell.

At 3 days and 4 days post-infection, 200 uL of cells were collected in a 96-well V-bottom plate. After centrifugation and removing supernatant, cells were lysed with 50 uL of 1× in-house cell lysis buffer for 20 min at RT before being transferred to a PCR plate. The cells were incubated at 56 C for 15 min and 10 min in a thermocycler. The lysate was then diluted 20× with water and run with Alb/WPRE primers to determine GFP %.

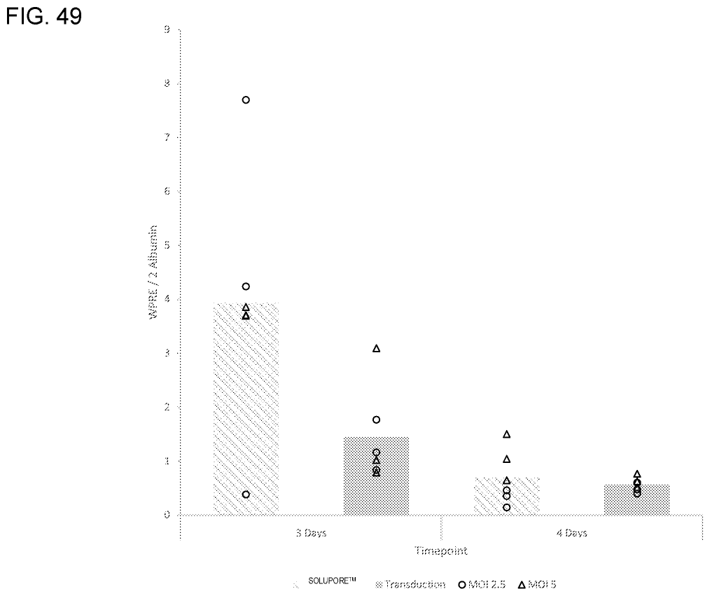

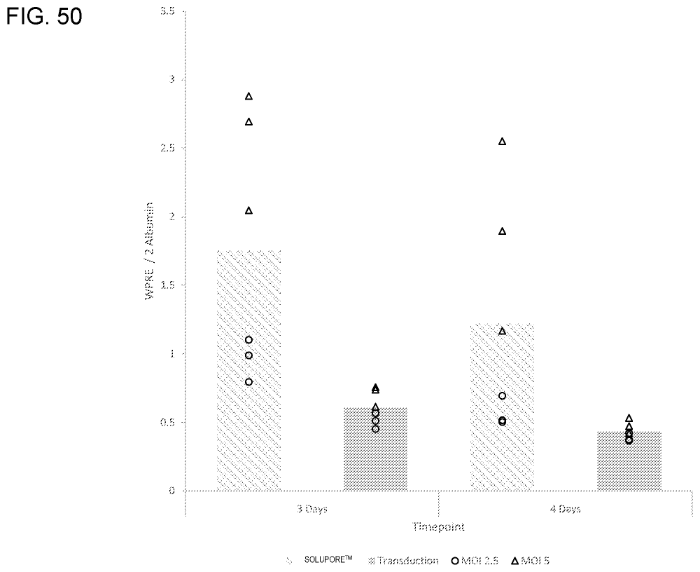

Estimated copies of GFP per cell (GFP %) based on WPRE per 2 albumin for Exp 1 (see FIG. 49 and FIG. 50).

The results indicate that a significant increase in GFP integrations was achieved using the SOLUPORE™ process compared to static transduction (on both day 3 and day 4) (see FIG. 49 and FIG. 50).

### Example 11: Viral Infection Process and Droplet Properties

The atomisation of lentivirus within the transfection chamber is a distinct process from the SOLUPORE™ process. As described herein, the cargo delivered to the population of cells is a virus (e.g., a lentivirus), that is biologically active and viable.

Typical titres of lentivirus range from 106 to 10⁷ transducing units per milliliter (TU/ml) and the consistency of lentivirus at these concentrations is highly, dynamically viscous relative to water/ethanol mixtures. For illustration, (Table 1) the dynamic viscosity of water at room temperature is close to 1 mPa s, the dynamic viscosity of ethanol is close to 0.1 mPa s, the dynamic viscosity of olive oil is close to 60 0.1 mPa s and the dynamic viscosity of castor oil is close to 600 0.1 mPa s. The dynamic viscosity of lentivirus was reported by Tran, Reginald, PhD Thesis, Georgia Tech (2016), as 6913 mPas, being close to being a log more viscous than castor oil. Sterile filtered 1% bovine serum albumin (BSA) has been found by some to decrease molecular interactions that might lead the virus particles to “stick” to the injection apparatus. (Jasnow A. et al. Methods Mol Biol. “Construction of Cell-Type Specific Promoter Lentiviruses for Optically Guiding Electrophysiological Recordings and for Targeted Gene Delivery” 2009; 515: 199-213, incorporated herein by reference in its entirety).

Dynamic Viscosity is an important factor in atomisation. Experimental studies on atomization in an internal-mixing twin-fluid atomizer, such as that used in the SOLUPORE™ process, over a wide range of liquid viscosity, gas supply pressure and Gas to Liquid mass Ratio (GLR) have been performed. See, e.g., Li, Z. et al. “Effect of liquid viscosity on atomization in an internal-mixing twin-fluid atomizer” Fuel vol. 103; January 2013 pages 486-494, incorporated herein by reference in its entirety. Among all test conditions, the finest sprays were obtained at an axial distance of 150 mm. However, droplet size distributions notably changed when viscosity increased to 120 mPa s. The higher viscosity droplets produced larger droplets (e.g., 1 to 2 logs larger than the current droplets produced by the SOLUPORE™ processed measured droplet size distribution, FIG. 51). The larger droplets represented a large proportion of the droplet population (distribution), and the decay of droplet velocities along the spray axis was stronger at a larger viscosity.

A table showing the dynamic viscosities of common liquids is shown below (and graph provided at FIG. 52).

Absolute or dynamic viscosities for some common liquids at temperature 300 K are indicated below:

| | Absolute Viscosity | | |
|---|---|---|---|
| Fluid | (Ns/m², Pa s) | (centipoise, cP) (10⁻⁴ lb/s ft) | |
| Acetic acid | 0.001155 | 1.155 | 7.76 |
| Acetone | 0.000316 | 0.316 | 2.12 |
| Alcohol, ethyl (ethanol) | 0.001095 | 1.095 | 7.36 |
| Alcohol, methyl (methanol) | 0.00056 | 0.56 | 3.76 |
| Alcohol, propyl | 0.00192 | 1.92 | 12.9 |
| Benzene | 0.000601 | 0.601 | 4.04 |
| Blood | 0.003-0.004 | | |
| Bromine | 0.00095 | 0.95 | 6.38 |
| Carbon Disulfide | 0.00036 | 0.36 | 2.42 |
| Carbon Tetrachloride | 0.00091 | 0.91 | 6.11 |
| Castor Oil | 0.650 | 650 | |
| Chloroform | 0.00053 | 0.53 | 3.56 |
| Decane | 0.000859 | 0.859 | 5.77 |
| Dodecane | 0.00134 | 1.374 | 9.23 |
| Ether | 0.000223 | 0.223 | 1.50 |
| Ethylene Glycol | 0.0162 | 16.2 | 109 |
| Trichlorofluoromethane refrigerant R-11 | 0.00042 | 0.42 | 2.82 |
| Glycerine | 0.950 | 950 | 6380 |
| Heptane | 0.000376 | 0.376 | 2.53 |
| Hexane | 0.000297 | 0.297 | 2.00 |
| Kerosene | 0.00164 | 1.64 | 11.0 |
| Linseed Oil | 0.0331 | 33.1 | 222 |
| Mercury | 0.0015 | 1.53 | 10.3 |
| Milk | 0.003 | | |
| Octane | 0.00051 | 0.51 | 3.43 |
| Phenol | 0.0080 | 8.0 | 54 |
| Propane | 0.00011 | 0.11 | 0.74 |
| Propylene | 0.00009 | 0.09 | 0.60 |
| Propylene glycol | 0.042 | 42 | |
| Toluene | 0.000550 | 0.550 | 3.70 |
| Turpentine | 0.001375 | 1.375 | 9.24 |
| Water, Fresh | 0.00089 | 0.89 | 6.0 |

It can be concluded from that the atomisation of lentivirus within the SOLUPORE™ process produced larger, slower moving droplets and the experience of the layer of cells beneath, are different form the previously described SOLUPORE™ process. Consequently, this atomisation process resulted in transfection levels of close to 30%, a surprising and unexpected observation.

### Viral Infection Process

The dynamic viscosity of water is close to 1 mPa s (milli Pascale seconds). The dynamic viscosity of ethanol/water mixes is also close to 1 mPa s. The dynamic viscosity of an aqueous solution that can include an ethanol concentration of 5 to 30%. The aqueous solution can include one or more of 75 to 98% H₂O, 2 to 45% ethanol, 6 to 91 mM sucrose, 2 to 35 mM KCl, 2 to 35 mM ammonium acetate, and 1 to 14 mM (4-(2-hydroxyethyl)-1-piperazineethanesulfonic acid) (HEPES) has a viscosity is the region of 2 mPa s.

The dynamic viscosity of lentivirus at titres 10\hat{ }7 to 10\hat{ }8 TU/mL is close to 6913 mPa s. As the viscosity of a fluid increases, at a given spray pressure, for example 1.7 bar, it will tend to form larger droplets when sprayed. Sprays consisting of smaller droplets have a much larger surface area per volume than those made up of larger droplets. Moreover, the droplets have a lower surface tension than water, and thus the droplets get even larger. In turn, the cells experience an entirely different process. As such finer sprays are better able to spread out on their target surface. This effect is relatively small for fluids with viscosities below 10 mPa s but becomes more pronounced with higher dynamic viscosities. Fluids with higher dynamic viscosities than water or water/ethanol mixes will have higher mean droplet sizes for any given flow rate and pressure. The interplay between the mechanical properties of fluids can be calculated by the generally accepted formula;

$${D}_{f} = {D}_{w} {V}_{f}^{0.2} \qquad Equation [ 1 ]$$

Where D_{f}=modified droplet size for the fluid in question
D_{w}=Droplet size calculated for water
V_{f}=the viscosity of the fluid (viscosity in mPa s; water=1.0 mPA s, lentivirus is 6913 mPa s)

It can be calculated from Equation [1] that Lentivirus droplets (e.g., droplets including a volume of aqueous solution including a virus, an ethanol concentration of 5 to 30% and one or more of 75 to 98% H₂O, 2 to 45% ethanol, 6 to 91 mM sucrose, 2 to 35 mM KCl, 2 to 35 mM ammonium acetate, and 1 to 14 mM (4-(2-hydroxyethyl)-1-piperazineethanesulfonic acid) (HEPES)) sprayed under the same pressure and flow conditions as water/ethanol mixes will have droplet sizes close to 5.9 times larger than the water/ethanol droplets. As described in International Application WO 2016/065341 (incorporated herein by reference in its entirety), droplets in the size range of 30 μm to 100 μm and 50 μm to 80 μm in diameter were described. Generally, in the methods described herein if the aqueous solution being sprayed includes a virus (e.g., a lentivirus), the droplet size range is about 150 μm to 600 μm in diameter, or about 177 μm to 590 μm in diameter. In other examples, the droplet diameter size is 200 μm to 600 μm, or about 300 μm to 600 μm, or about 400 μm to 600 μm, or about 500 μm to 600 μm. In other examples, the droplet size of the invention herein may be larger than 600 μm, for example about 600 μm to 1000 μm in diameter, or about 600 μm to 900 μm, or about 600 μm to 800 μm, or about 600 μm to 700 μm in diameter. In some examples droplet size may be characterized by a diameter of up to 1000 μm, e.g., 150 μm to 1000 μm.

These droplets are much larger than anticipated or described in WO 2016/065341, which “A portion of the colloidal droplets produced can be too large for a given intracellular delivery application. Because a portion of the colloidal droplets produced are too large, cell death may occur notwithstanding the production of appropriately sized colloidal droplets.” See WO 2016/065341 at ¶ [0172]. Accordingly, it was unexpected and surprising that the cells (e.g., cells contacted with an aqueous solution including a virus) tolerated such a different process as compared to the SOLUPRE™ process described in WO 2016/065341, and the cells were infected with lentivirus and viable.

### Droplet Size Properties

The larger diameter droplets of the invention described herein have a larger volume and weight, travel more slowly and impact the cell layer with greater force. For example the volume of a droplet increases by a factor of close to 206.8 when the diameter increases by a factor of 5.9. Thus, the fluid mechanics of this system are distinct from those described in See WO 2016/065341 and constitute a new viral infection process.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed:

1. A method of delivering a virus across a plasma membrane of a cell, comprising, providing a population cells and contacting the population of cells with a volume of an isotonic aqueous solution, said solution comprising said virus and an alcohol at a concentration of greater than 2%, wherein contacting said population of cells with the volume of aqueous solution is performed by gas propelling the aqueous solution to form a spray, said spray comprising a droplet comprising a diameter of greater than or equal to 150 μm.

2. The method of claim 1, wherein said spray comprises a droplet comprising a diameter in the range of 177 μm to 590 μm.

3. The method of claim 1, wherein said virus comprises a lentivirus, a retrovirus, an adenovirus, an adeno-associated virus (AAV), or a herpes simplex virus (HSV).

4. The method of claim 1, wherein said virus is a lentivirus.

5. The method of claim 1, wherein the population of cells comprises mammalian cells.

6. The method of claim 1, wherein the population of cells comprises adherent or suspension cells.

7. The method of claim 6, wherein the population of cells comprises a non-adherent cell.

8. The method of claim 7, wherein said non-adherent cell comprises a T lymphocyte.

9. The method of claim 3, wherein a transduction efficiency is at least 30%, at least 40%, at least 50%, or at least 60%.

10. The method of claim 6, wherein the population of cells comprises HEK293 cells, HEK293T cells, Lenti-x 293T cells, or HEK293F cells.

11. The method of claim 1, wherein said population of cells comprises a natural killer cell.

12. The method of claim 1, wherein said alcohol comprises ethanol.

13. The method of claim 1, wherein said aqueous solution comprises greater than 2% ethanol.

14. The method of claim 1, wherein said aqueous solution comprises greater than 10% ethanol.

15. The method of claim 1, wherein said aqueous solution comprises between 20-30% ethanol.

16. The method of claim 1, wherein said aqueous solution comprises an ethanol concentration of 5 to 30%.

17. The method of claim 1, wherein the aqueous solution further comprises one or more of the following components: 75 to 98% H₂O, 2 to 45% ethanol, 6 to 91 mM sucrose, 2 to 35 mM KCl, 2 to 35 mM ammonium acetate, and 1 to 14 mM (4-(2-hydroxyethyl)-1-piperazineethanesulfonic acid) (HEPES).

18. The method of claim 1, wherein said aqueous solution comprises: Sucrose 32.5 mM, KCl 106 mM, Hepes 5 mM, and Ethanol 12% v/v.

19. The method of claim 1, wherein said population of cells comprises a layer of non-adherent cells on a substrate.

20. The method of claim 1, wherein said layer resides on a membrane filter.
